SEQUENCE LISTING (16 sequences)

SEQ ID NO: 1 - protein, 5 aa, Artificial
  Motif
LPXTG

SEQ ID NO: 2 - protein, 35 aa, S. aureus
LPETGEENPFIGTTVFGGLSLALGAALLAGRRREL

SEQ ID NO: 3 - protein, 37 aa, S. aureus
LPETGGEESTNKGMLFGGLFSILGLALLRRNKKNHKA

SEQ ID NO: 4 - protein, 34 aa, S. sobrinus
LPATGDSSNAYLPLLGLVSLTAGFSLLGLRRKQD

SEQ ID NO: 5 - protein, 36 aa, E. faecalis
LPKTGEKQNVLLTVVGSLAAMLGLAGLGFKRRKETK

SEQ ID NO: 6 - protein, 34 aa, S. pyogenes
LPSTGSIGTYLFKAIGSAAMIGAIGIYIVKRRKA

SEQ ID NO: 7 - protein, 34 aa, L. monocytogenes
LPTTGDSDNALYLLLGLLAVGTAMALTKKARASK

SEQ ID NO: 8 - protein, 38 aa, A. viscosus
LPLTGANGVIFLTIAGALLVAGGAVVAYANKRRHVAKH

SEQ ID NO: 9 - protein, 33 aa, S. agalactiae
LPYTGVASNLVLEIMGLLGLIGTSFIAMKRRKS

SEQ ID NO: 10 - protein, 33 aa, S. aureus
LPKTGMKIITSWITWVFIGILGLYLILRKRFNS

SEQ ID NO: 11 - protein, 32 aa, S. mutans
LPSTGEQAGLLLTTVGLVIVAVAGVYFYRTRR

SEQ ID NO: 12 - protein, 35 aa, S. pyogenes
LPSTGETANPFFTAAALTVMATAGVAAVVKRKEEN

SEQ ID NO: 13 - protein, 5 aa, Artificial
  Motif
NPQTN

SEQ ID NO: 14 - protein, 6 aa, Artificial
  Motif
LPXTGG

SEQ ID NO: 15 - protein, 7 aa, Artificial
  Motif
LPXTAST

SEQ ID NO: 16 - protein, 5 aa, Artificial
  Motif
LAXTG

(19) United States
(12) Patent Application Publication
(10) Pub. No.: US 2016/0002645 A1
(43) Pub. Date: Jan. 7, 2016

(54) RECOMBINANT BACILLUS SUBTILIS THAT CAN GROW ON PLANT BIOMASS

(71) Applicant: THE REGENTS OF THE UNIVERSITY OF CALIFORNIA, Oakland, CA (US)

(72) Inventors: Robert T. Clubb, Culver City, CA (US); Timothy Anderson, St. Louis, MO (US)

(21) Appl. No.: 14/443,031

(22) PCT Filed: Nov. 15, 2013
(86) PCT No.: PCT/US2013/070393

§ 371 Date: May 14, 2015

Related U.S. Application Data
(60) Provisional application No. 61/727,601, filed on Nov. 16, 2012.

(51) Int. Cl. C12N 15/75; C12P 19/02; C12P 19/14

(52) U.S. Cl. CPC C12N 15/75; C12P 19/14; C12P 19/02

(57) ABSTRACT

In certain embodiments recombinant modified microorganisms (e.g., Gram-positive bacteria, yeasts, etc.) are provided that display on their surface a minicellulosome comprising two or more cellulolytic enzymes where the minicellulosome is self-assembled by the microorganism and resulting microorganism is capable of growing on untreated plant biomass (e.g. biomass that is not acid treated and/or enzymatically pre-digested). In certain embodiments the microorganism grows on lignocellulose as the sole carbon source.

### CROSS-REFERENCE TO RELATED APPLICATIONS

This application claims priority to and benefit of U.S. Ser. No. 61/727,601, filed on Nov. 16, 2012, which is incorporated herein by reference in its entirety for all purposes.

### STATEMENT OF GOVERNMENTAL SUPPORT

This invention was made with Government support of Grant No: DE-FC02-02ER63421, awarded by the U.S. Department of Energy. The Government has certain rights in this invention.

### BACKGROUND

Petroleum-based fuels and commodities are commonplace, and their widespread use is growing despite evidence that the earth's petroleum resources are dwindling (Kerr (2008) Science 322: 1178-1179). It is therefore desirable to find renewable sources of carbon that can be used as an alternative to petroleum. Lignocellulosic biomass is an obvious choice since it constitutes more than half of the organic carbon in the biosphere (Boerjan et al. (2003) Ann. Rev. Plant Biol., 54: 519-546; Reddy and Yang (2005) Trends in Bbiotechnology 23: 22-27; Werpy et al. (2004) Top Value Added Chemicals From Biomass. Volume 1-Results of Screening for Potential Candidates From Sugars and Synthesis Gas. DTIC Institution.). A major obstacle to its cost effective commercialization, however, is its recalcitrance to hydrolysis into fermentable sugars (primarily glucose and xylose) (Lynd et al. (2005) Curr. Opin. Biotechnol. 16: 577-583; Mielenz (2001) Curr. Opin. Microbiol. 4: 324-329). Many currently used industrial methods degrade lignocellulose using a two-step process in which it is thermochemically pretreated and then hydrolyzed using enzymes produced by Trichoderma reesei. While high yields can be obtained using this approach, it can be costly and inefficient (Wilson (2009) Curr. Opin. Biotechnol. 20: 295-299; Himmel et al. (2007) Science 315: 804-807; Hendriks and Zeeman (2009) Bioresource Technol., 100: 10-18; Zhao et al. (2009) Appl. Microbiol. Biotechnol. 82: 815-827; Yeoman et al. (2010) Adv. Appl. Microbiol. 70: 1-55; Miller and Blum (2010) Environmental Technol., 31: 1005-1015). The creation of recombinant microbes that can degrade biomass efficiently is an attractive alternative to currently used methods. It is also an essential step towards the creation of a consolidated bioprocessor (CBP), a single microbe that has the capacity to convert lignocellulose into valuable end products such as ethanol (Olson et al. (2012) Curr. Opin. Biotechnol., 23: 396-405; Zhang and Zhang (2010) Engineering in Life Sci., 10: 398-406; Liao et al. (2011) Biotechnol. J. 6: 1409-1418; Lynd et al. (2008) Nature Biotechnol., 26: 169-172; la Grange et al. (2010) Appl. Microbiol. Biotechnol., 87: 1195-1208).

Lignocellulose consists of cellulose and hemicellulose polymers that are surrounded by lignin (Harris and DeBolt (2010) Plant Biotechnol. J. 8: 244-262; Carroll and Somerville (2009) Ann. Rev. Plant Biol., 60: 165-182). Cellulose is a homopolymer of beta-1,4 linked glucose monomers, which hydrogen bond with similar polymers to form both crystalline and amorphous regions. The crystalline regions are in part degraded by exoglucanases, which act on either the reducing or non-reducing ends of the cellulose polymer (Ghose (1977) Adv. Biochem. Engineer., Vol. 6. Springer Berlin/Heidelberg). The amorphous regions within cellulose are less ordered and are accessible to endoglucanases that cleave internal beta-1,4-glucosidic bonds. Endoglucanases also cleave chains within the crystalline region, but at a much slower rate (Id.). Hemicellulose, on the other hand, is a heteropolymer with relatively high xylan content (Pauly and Keegstra (2010) Curr. Opin. Plant Biol., 13: 305-312). It has an amorphous structure that can be easily hydrolyzed by acid or base, but enzymatic degradation requires several hemicellulase enzymes, including exoxylanases and endoxylanases (Banerjee et al. (2010) Biotechnol. Bioengineer., 106: 707-720; McCann and Carpita (2008) Curr. Opin. Plant Biol., 11: 314-320). Lignin also contributes substantially to the hydrolytic recalcitrance of lignocellulose as this extremely complex polymer consists of many types of monomers connected by a diverse array of covalent linkages (Boerjan et al. (2003) Ann. Rev. Plant Biol., 54: 519-546; Wardrop (1969) Australian J. Botany 17: 229-240).

Despite its complexity, several naturally occurring microorganisms have evolved the capacity to efficiently break down lignocellulose and use it as a nutrient (Ransom-Jones et al. (2012) Microbial Ecol., 63: 267-281; Wilson (2011) Curr. Opin. Microbiol., 14: 259-263). Significantly, anaerobic and aerobic microorganisms use different strategies to degrade lignocellulose. Aerobic fungi secrete enzymes with different cellulolytic activities, whereas anaerobic bacteria incorporate cellulases into a cell-surface displayed super-structure known as a cellulosome (Miller and Blum (2010) Environmental Technol., 31: 1005-1015; Doi and Kosugi (2004) Microbiology 2: 541-551; Doi (2008) Ann. New York Acad. Sci., 1125: 267-279; Bayer et al. (2004) Ann. Rev. Microbiol., 58: 521-554; Ding et al. (2008) Curr. Opin. Biotechnol., 19: 218-227). Although their architectures vary, cellulosomes from different microbes consist of a backbone scaffoldin protein that contains several cohesin modules capable of non-covalently binding in a 1:1 ratio to the dockerin modules of the cellulase enzymes. By clustering the cellulases into a cellulosome, the microbe is able to increase the effective enzyme concentration near its cell surface and to combine many enzymes with different activities into a single complex, enabling them to function synergistically (Bayer et al. (1998) Curr. Opin. Structural Biol., 8: 548-557). Although these organisms have potent cellulolytic activity, most are unattractive candidates for use as a CBP as they are difficult to genetically manipulate or cultivate.

Towards the goal of creating a robust CBP microbe, two model microorganisms (Bacillus subtilis and Saccharomyces cerevisiae) have been engineered to display small artificial cellulosomes (i.e., minicellulosomes) (Lilly et al. (2009) FEMS Yeast Res., 9: 1236-1249; Anderson et al. (2011) Appl. Environ. Microbiol., 77: 4849-4858; Steen et al. (2010) Nature 463: 559-562; You et al. (2012) Appl. Environ. Microbiol., 78: 1437-1444; Tsai et al. (2009) Appl. Environ. Microbiol., 75: 6087-6093; Fan et al. (2012) Proc. Natl. Acad. Sci. USA, 109: 13260-13265). In most of these systems, a miniscaffoldin containing one or more cohesin modules is covalently or non-covalently attached to the cell surface. The minicellulosome is then often assembled ex vivo by adding purified cellulase enzymes that are fused to dockerin modules. While these recombinant microorganisms are able to degrade amorphous purified cellulose (e.g., regenerated amorphous cellulose (RAC), phosphoric acid swollen cellulose) or soluble cellulose (e.g., carboxymethyl cellulose (CMC)), their ability to degrade industrially relevant forms of biomass such as corn stover, switchgrass, and straw has not been demonstrated. Moreover, the requirement for ex vivo assembly of their cellulosomes can make some of these microbes impractical for use as an industrial CBP.

### SUMMARY

In certain embodiments recombinant modified microorganisms (e.g., Gram-positive bacteria, etc.) are provided that display on their surface a minicellulosome comprising two or more cellulolytic enzymes where the minicellulosome is self-assembled by the microorganism and resulting microorganism is capable of growing on untreated biomass (e.g. biomass that is not acid treated and/or enzymatically pre-digested). In certain embodiments the microorganism grows on lignocellulose as the sole carbon source.

In one illustrative embodiment a recombinant modified Gram-positive bacterium is provided that displays on its surface a minicellulosome comprising two or more cellulolytic enzymes, where the bacterium comprises: a protein encoding two or more cohesin domains wherein said protein is covalently linked to the surface of said microorganism, and wherein each of said cohesin domains is docked to a dockerin attached to a cellulolytic enzyme; and the one or more constructs that encode dockerin(s) attached to said cellulolytic enzyme(s); and the minicellulosome is self-assembled by said bacterium.

In various aspects, the invention(s) contemplated herein may include, but need not be limited to, any one or more of the following embodiments:

### Embodiment 1

A recombinant modified Gram-positive bacterium that displays on its surface a minicellulosome including two or more cellulolytic enzymes, wherein said bacterium includes: a protein encoding two or more cohesin domains wherein said protein is covalently linked to the surface of said microorganism, and wherein each of said cohesin domains is docked to a dockerin attached to a cellulolytic enzyme; and said bacterium includes one or more constructs that encode said dockerin(s) attached to said cellulolytic enzyme(s); and wherein said minicellulosome is self-assembled by said bacterium.

### Embodiment 2

The bacterium of embodiment 1, wherein said bacterium grows on untreated biomass.

### Embodiment 3

The bacterium according to any one of embodiments 1-2, wherein said bacterium grows on lignocellulose as the sole carbon source.

### Embodiment 4

The bacterium according to any one of embodiments 1-3, wherein said minicellulosome includes at least three cellulolytic enzymes and all of said enzymes are encoded by said bacterium.

### Embodiment 5

The bacterium according to any one of embodiments 1-4, wherein said protein encoding two or more cohesin domains includes a secretory signal sequence at the N-terminus and a cell wall sorting signal (CWSS) at the carboxyl terminus.

### Embodiment 6

The bacterium of embodiment 5, wherein said cell wall sorting signal includes an LPXTG motif.

### Embodiment 7

The bacterium of embodiment 5, wherein said cell wall sorting signal includes a sequence shown in Table 1.

### Embodiment 8

The bacterium of embodiment 5, wherein said cell wall sorting signal includes a CWSS from Staphylococcus aureus fibronectin binding protein B.

### Embodiment 9

The bacterium according to any one of embodiments 5-8, wherein said secretory signal sequence includes a B. subtilis phrC secretory signal or homologues thereof.

### Embodiment 10

The bacterium of embodiment 9, wherein said secretory signal sequence includes a secretion signal derived from B. subtilis phrC.

### Embodiment 11

The bacterium according to any one of embodiments 1-10, wherein said protein encoding two or more cohesin domains encodes a carbohydrate binding module (CBM).

### Embodiment 12

The bacterium of embodiment 11, wherein said carbohydrate binding module is a family 3 carbohydrate binding module.

### Embodiment 13

The bacterium according to any one of embodiments 1-12, wherein said two or more cohesin domains are type I cohesin modules.

### Embodiment 14

The bacterium according to any one of embodiments 1-13, wherein said two or more cohesin domains are cohesin domains from different microorganisms.

### Embodiment 15

The bacterium according to any one of embodiments 1-14, wherein said two or more cohesin domains includes a cohesin domain from an organism selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 16

The bacterium according to any one of embodiments 1-15, wherein said two or more cohesin domains comprise cohesin domains from two organisms selected from the group consisting of C. thermocellum (t), C. cellulolyticum (c) and R. flavefaciens (f).

### Embodiment 17

The bacterium according to any one of embodiments 1-16, wherein said two or more cohesin domains comprise cohesin domains from C. thermocellum (t), C. cellulolyticum (c) and R. flavefaciens (f).

### Embodiment 18

The bacterium according to any one of embodiments 1-15, wherein said dockerins comprise one or more dockerin domains from organism(s) selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 19

The bacterium according to any one of embodiments 1-18, wherein said dockerins dockerin domains from two organisms selected from the group consisting of C. thermocellum (t), C. cellulolyticum (c) and R. flavefaciens (f).

### Embodiment 20

The bacterium according to any one of embodiments 1-19, wherein said dockerins comprise dockerins from C. thermocellum (t), C. cellulolyticum (c) and R. flavefaciens (f).

### Embodiment 21

The bacterium according to any one of embodiments 1-20, wherein said cellulolytic enzyme(s) on dormant bacteria are stable for at least 1 day, more preferably for at least 2 days, and most preferably at least 3 days.

### Embodiment 22

The bacterium according to any one of embodiments 1-21, wherein said cellulolytic enzyme(s) comprise one or more enzymes selected from the group consisting of an endocellulase, an exocellulase, a beta-glucosidase (cellobiase), an oxidative cellulase, a xylanase, a hemicellulase, a lichenase, a chitenase, and a cellulose phosphorylase.

### Embodiment 23

The bacterium according to any one of embodiments 1-22, wherein said minicellulosome includes at least two different cellulolytic enzymes.

### Embodiment 24

The bacterium according to any one of embodiments 1-23, wherein said minicellulosome includes at least three different cellulolytic enzymes.

### Embodiment 25

The bacterium according to any one of embodiments 1-24, wherein said minicellulosome includes at least one endoglucanase.

### Embodiment 26

The bacterium according to any one of embodiments 1-25, wherein said minicellulsome includes at least one exoglucanase.

### Embodiment 27

The bacterium according to any one of embodiments 1-26, wherein said minicellulsome includes at least two endoglucanases and at least one exoglucanase.

### Embodiment 28

The bacterium according to any one of embodiments 1-27, wherein said minicellulosome includes Clostridium cellulolyticum endoglucanase Cel5A.

### Embodiment 29

The bacterium according to any one of embodiments 1-28, wherein said minicellulosome includes C. cellulolyticum endoglucanase Cel48F.

### Embodiment 30

The bacterium according to any one of embodiments 1-29, wherein said minicellulosome includes C. cellulolyticum exoglucanase Cel9E 31:

### Embodiment 31

The bacterium according to any one of embodiments 1-30, wherein said Gram-positive bacterium includes a Gram-positive bacterium that encodes a sortase.

### Embodiment 32

The bacterium of embodiment 31, wherein said Gram-positive bacterium includes a Gram-positive bacillus.

### Embodiment 33

The bacterium of embodiment 32, wherein said Gram-positive bacterium includes a genus selected from the group consisting of Corynebacterium, Clostridium, Listeria, and Bacillus.

### Embodiment 34

The bacterium of embodiment 33, wherein said bacterium is a Clostridium acetobutylicum.

### Embodiment 35

The bacterium of embodiment 33, wherein said Gram-positive bacterium comprise is B. subtilis.

### Embodiment 36

The bacterium of embodiment 31, wherein said Gram-positive bacterium includes a thermophilic Geobacillus spp.

### Embodiment 37

The bacterium of embodiment 31, wherein said Gram-positive bacterium includes a Gram-positive coccus.

### Embodiment 38

The bacterium of embodiment 37, wherein said bacterium is selected from the group consisting of S. aureus, S. epidermis, and S. saprophyticus.

### Embodiment 39

A recombinant modified Gram-positive bacterium that displays on its surface a minicellulosome including two or more cellulolytic enzymes, wherein said bacterium includes (e.g., the minicellulosome includes): a first protein encoding one or more cohesin domains wherein said protein is covalently linked to the surface of said microorganism, and wherein each of one or more cohesin domains is docked to a dockerin attached to a cellulolytic enzyme and said second protein additionally encodes a linking dockerin or a linking cohesin; a second protein encoding one or more cohesin domains wherein each of one or more cohesin domains is docked to a dockerin attached to a cellulolytic enzyme and said second protein additionally encodes a linking dockerin or a linking cohesin; wherein said second protein is docked to said first protein by a dockerin/cohesin interaction between said linking dockerin or linking cohesin encoded by said second protein and said linking dockerin or linking cohesin encoded by said first protein, where when said linking dockerin or linking cohesin on said first protein is a linking cohesin, said linking dockerin or linking cohesin on said second protein is a linking dockerin, and when said linking dockerin or linking cohesin on said first protein is a linking dockerin, said linking dockerin or linking cohesin on said second protein is a linking cohesin.

### Embodiment 40

The bacterium of embodiment 39, wherein said first protein encodes a linking cohesin and said second protein encodes a linking dockerin and said second protein is attached to said first protein by a dockerin/cohesin interaction between said linking cohesin on said first protein and said linking dockerin on said second protein.

### Embodiment 41

The bacterium of embodiment 39, wherein said first protein encodes a linking dockerin and said second protein encodes a linking cohesin and said second protein is attached to said first protein by a dockerin/cohesin interaction between said linking dockerin on said first protein and said linking cohesin on said second protein.

### Embodiment 42

The bacterium according to any one of embodiments 39-41, wherein said one or more cohesin domains in said first protein comprise at least two cohesin domains each docked to a cellulolytic enzyme attached to a dockerin.

### Embodiment 43

The bacterium according to any one of embodiments 39-42, wherein said one or more cohesin domains in said first protein comprise at least three cohesin domains each docked to a cellulolytic enzyme attached to a dockerin.

### Embodiment 44

The bacterium according to any one of embodiments 39-43, wherein said one or more cohesin domains in said second protein comprise at least two cohesin domains each docked to a cellulolytic enzyme attached to a dockerin.

### Embodiment 45

The bacterium of embodiment 44, wherein said one or more cohesin domains in said second protein comprise at least three cohesin domains each docked to a cellulolytic enzyme attached to a dockerin.

### Embodiment 46

The bacterium according to any one of embodiments 39-45, wherein: said bacterium includes a third protein encoding one or more cohesin domains wherein each of one or more cohesin domains is docked to a dockerin attached to a cellulolytic enzyme and said third protein additionally encodes a linking dockerin or a linking cohesin; said second protein includes a second linking dockerin or a second linking cohesin; wherein said third protein is docked to said second protein by a dockerin/cohesin interaction between said second linking dockerin or second linking cohesin encoded by said second protein and said linking dockerin or linking cohesin encoded by said third protein, where when said second linking dockerin or linking cohesin on said second protein is a linking cohesin, said linking dockerin or linking cohesin on said third protein is a linking dockerin, and when said second linking dockerin or linking cohesin on said second protein is a linking dockerin, said linking dockerin or linking cohesin on said second protein is a linking cohesin.

### Embodiment 47

The bacterium of embodiments 46, wherein said second linking dockerin or linking cohesin on said second protein is a second linking cohesin, said linking dockerin or linking cohesin on said third protein is a linking dockerin and said third protein is attached to said second protein by a dockerin/cohesin interaction between said second linking cohesin on said second protein and said linking dockerin on said third protein.

### Embodiment 48

The bacterium of embodiment 46, wherein said second linking dockerin or linking cohesin on said second protein is a linking dockerin and said linking dockerin or linking cohesin on said third protein is a linking cohesin and said third protein is attached to said second protein by a dockerin/cohesin interaction between said second linking dockerin on said second protein and said linking cohesin on said third protein.

### Embodiment 49

The bacterium according to any one of embodiments 46-48, wherein said one or more cohesin domains in said third protein comprise at least two cohesin domains each docked to a cellulolytic enzyme attached to a dockerin.

### Embodiment 50

The bacterium of embodiment 49, wherein said one or more cohesin domains in said third protein comprise at least three cohesin domains each docked to a cellulolytic enzyme attached to a dockerin.

### Embodiment 51

The bacterium according to any one of embodiments 39-50, wherein said cellulases form a cellulosome that self assembles on said bacterium.

### Embodiment 52

The bacterium according to any one of embodiments 39-51, wherein said bacterium grows on untreated plant biomass.

### Embodiment 53

The bacterium according to any one of embodiments 39-52, wherein said bacterium grows on lignocellulose as the sole carbon source.

### Embodiment 54

The bacterium according to any one of embodiments 39-53, wherein said minicellulosome includes at least three cellulolytic enzymes and all of said enzymes are encoded by said bacterium.

### Embodiment 55

The bacterium according to any one of embodiments 39-54, wherein one or more of the cohesin domains in said first protein that are docked to dockerin-bearing enzymes are Type-I cohesins.

### Embodiment 56

The bacterium according to any one of embodiments 39-55, wherein one or more of the cohesin domains in said second protein that are docked to dockerin-bearing enzymes are Type-I cohesins.

### Embodiment 57

The bacterium according to any one of embodiments 46-64, wherein one or more of the cohesin domains in said third protein that are docked to dockerin-bearing enzymes are Type-I cohesins.

### Embodiment 58

The bacterium according to any one of embodiments 39-57 wherein the linking dockerin/cohesins joinining said first protein to said second protein are Type II dockerins and cohesins.

### Embodiment 59

The bacterium according to any one of embodiments 39-57 wherein the linking dockerin/cohesins joining said second protein to said third protein, when said third protein is present, are Type II dockerins and cohesins.

### Embodiment 60

The bacterium according to any one of embodiments 39-59, wherein said first protein encoding two or more cohesin domains includes a secretory signal.

### Embodiment 61

The bacterium of embodiment 60, wherein said secretory signal sequence includes a B. subtilis phrC secretory signal or homologues thereof.

### Embodiment 62

The bacterium of embodiment 61, wherein said secretory signal sequence includes a secretion signal derived from B. subtilis phrC.

### Embodiment 63

The bacterium according to any one of embodiments 39-62, wherein said first protein encoding two or more cohesin domains includes a cell wall sorting signal (CWSS).

### Embodiment 64

The bacterium of embodiment 63, wherein said protein encoding two or more cohesin domains includes a secretory signal sequence at the N-terminus and a cell wall sorting signal (CWSS) at the carboxyl terminus.

### Embodiment 65

The bacterium according to any one of embodiments 63-64, wherein said cell wall sorting signal includes an LPXTG motif.

### Embodiment 66

The bacterium according to any one of embodiments 63-64, wherein said cell wall sorting signal includes a sequence shown in Table 1.

### Embodiment 67

The bacterium according to any one of embodiments 63-64, wherein said cell wall sorting signal includes a CWSS from Staphylococcus aureus fibronectin binding protein B.

### Embodiment 68

The bacterium according to any one of embodiments 39-67, wherein said first protein encoding two or more cohesin domains and/or said second protein encoding two or more cohesin domains encodes a carbohydrate binding module (CBM).

### Embodiment 69

The bacterium of embodiment 68, wherein said carbohydrate binding module is a family 3 carbohydrate binding module.

### Embodiment 70

The bacterium of embodiment 68, wherein said carbohydrate binding module is a carbohydrate binding module derived from C. thermocelllum CipA.

### Embodiment 71

The bacterium according to any one of embodiments 39-69, wherein said two or more cohesin domains including said first protein, and/or said two or more cohesin domains including said second protein and/or said two or more cohesin domains including said third protein, when present, are cohesin domains from different microorganisms.

### Embodiment 72

The bacterium according to any one of embodiments 39-71, wherein said two or more cohesin including said first protein, and/or said two or more cohesin domains including said second protein and/or said two or more cohesin domains including said third protein, when present, comprise a cohesin domain from an organism selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 73

The bacterium according to any one of embodiments 39-72, wherein said two or more cohesin including said first protein, and/or said two or more cohesin domains including said second protein and/or said two or more cohesin domains including said third protein, when present, comprise cohesin domains from two organisms selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 74

The bacterium according to any one of embodiments 39-73, wherein said two or more cohesin including said first protein, and/or said two or more cohesin domains including said second protein and/or said two or more cohesin domains including said third protein, when present, comprise cohesin domains from three organisms selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus

### Embodiment 75

The bacterium according to any one of embodiments 39-73, wherein said two or more cohesin including said first protein, and/or said two or more cohesin domains including said second protein and/or said two or more cohesin domains including said third protein, when present, comprise cohesin domains from C. thermocellum (t), C. cellulolyticum (c) and R. flavefaciens (f).

### Embodiment 76

The bacterium according to any one of embodiments 39-75, wherein the dockerins coupling said cellulolytic enzymes to the cohesins including said first protein and/or said second protein, and/or said third protein when present comprise one or more dockerin domains from organism(s) selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 77

The bacterium according to any one of embodiments 39-75, wherein the dockerins coupling said cellulolytic enzymes to the cohesins including said first protein and/or said second protein, and/or said third protein when present comprise two or more dockerin domains from organism(s) selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 78

The bacterium according to any one of embodiments 39-75, wherein the dockerins coupling said cellulolytic enzymes to the cohesins including said first protein and/or said second protein, and/or said third protein when present comprise three or more dockerin domains from organism(s) selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

### Embodiment 79

The bacterium according to any one of embodiments 39-75, wherein the dockerins coupling said cellulolytic enzymes to the cohesins including said first protein and/or said second protein, and/or said third protein when present comprise dockerin domains from C. thermocellum (t), C. cellulolyticum (c) and R. flavefaciens (f).

### Embodiment 80

The bacterium according to any one of embodiments 39-79, wherein said cellulolytic enzyme(s) on dormant bacteria are stable for at least 1 day, more preferably for at least 2 days, and most preferably at least 3 days.

### Embodiment 81

The bacterium according to any one of embodiments 39-80, wherein said cellulolytic enzyme(s) comprise one or more enzymes selected from the group consisting of an endocellulase, an exocellulase, a beta-glucosidase (cellobiase), an oxidative cellulase, a xylanase, a hemicellulase, a lichenase, a chitenase, a mannase, an exogluconase, an endoxylanase, exogluconase, and a cellulose phosphorylase.

### Embodiment 82

The bacterium according to any one of embodiments 39-81, wherein said minicellulosome includes at least two different cellulolytic enzymes, or at least 3 different cellulolytic enzymes, or at least 4 different cellulolytic enzymes, or at least 5 different cellulolytic enzymes, or at least 6 different cellulolytic enzymes.

### Embodiment 83

The bacterium according to any one of embodiments 39-82, wherein said minicellulosome includes at least 6 different cellulolytic enzymes.

### Embodiment 84

The bacterium according to any one of embodiments 39-83, wherein said minicellulosome includes at least one endoglucanase.

### Embodiment 85

The bacterium according to any one of embodiments 39-84, wherein said minicellulsome includes at least one exoglucanase.

### Embodiment 86

The bacterium according to any one of embodiments 39-85, wherein said minicellulsome includes at least two endoglucanases and at least one exoglucanase.

### Embodiment 87

The bacterium of embodiment 83, wherein said minicellulosome includes an endoglucanase, a xylanase, an exoglucanase, an endoxylanase, and a mannase.

### Embodiment 88

The bacterium according to any one of embodiments 82-83, wherein said minicellulosome includes Cel5A.

### Embodiment 89

The bacterium according to any one of embodiments 82-83 and 88, wherein said minincellulosome includes XynA.

### Embodiment 90

The bacterium according to any one of embodiments 82-83 and 88-89, wherein said minincellulosome includes Cel48F.

### Embodiment 91

The bacterium according to any one of embodiments 82-83 and 88-90, wherein said minincellulosome includes CelS.

### Embodiment 92

The bacterium according to any one of embodiments 82-83 and 88-91, wherein said minincellulosome includes Cel9E.

### Embodiment 93

The bacterium according to any one of embodiments 82-83 and 88-92, wherein said minincellulosome includes Man5A.

### Embodiment 94

The bacterium according to any one of embodiments 39-93, wherein said Gram-positive bacterium includes a Gram-positive bacterium that encodes a sortase.

### Embodiment 95

The bacterium of embodiment 94, wherein said Gram-positive bacterium includes a Gram-positive bacillus.

### Embodiment 96

The bacterium of embodiment 95, wherein said Gram-positive bacterium includes a genus selected from the group consisting of Corynebacterium, Clostridium, Listeria, and Bacillus.

### Embodiment 97

The bacterium of embodiment 96, wherein said bacterium is a Clostridium acetobutylicum.

### Embodiment 98

The bacterium of embodiment 96, wherein said Gram-positive bacterium comprise is B. subtilis.

### Embodiment 99

The bacterium of embodiment 94, wherein said Gram-positive bacterium includes a thermophilic Geobacillus spp.

### Embodiment 100

The bacterium of embodiment 94, wherein said Gram-positive bacterium includes a Gram-positive coccus.

### Embodiment 101

The bacterium of embodiment 100, wherein said bacterium is selected from the group consisting of S. aureus, S. epidermis, and S. saprophyticus.

### Embodiment 102

A method of degrading cellulosic biomass into fermentable sugars, said method including: contacting said cellulosic biomass with a bacterium according to any one of embodiments 1-101, under conditions in which said bacteria partially or fully degrade cellulose in said cellulosic biomass to form one or more fermentable sugars.

### Embodiment 103

The method of embodiment 102, wherein said contacting includes contacting dormant bacteria to said cellulosic biomass.

### Embodiment 104

The method of embodiment 102, wherein said contacting includes culturing said bacteria with said cellulosic biomass.

### Embodiment 105

The method according to any one of embodiments 102-104, wherein said cellulosic biomass includes lignocellulosic biomass.

### Embodiment 106

The method according to any one of embodiments 102-105, wherein said cellulosic biomass comprise one or more materials selected from the group consisting of an agricultural plant waste (e.g., corn stover, cereal straw, sugarcane bagasse), a plant waste from an industrial process (e.g., sawdust, paper pulp), and a non-food energy crop (e.g., switchgrass).

### Embodiment 107

The method of embodiment 106, wherein said cellulosic biomass includes one or more materials selected from the group consisting of grasses, rice hulls, bagasse, jute, hemp, flax, bamboo, sisal, abaca, straw, corn cobs, corn stover, alfalfa, hay, coconut hair, seaweed, and algae.

### Embodiment 108

The method according to any one of embodiments 102-106, wherein said cellulosic biomass is not acidified and/or enzymatically pre-digested.

### Embodiment 109

A consolidated bioreactor for the conversion of a lignocellulosic biomass into bioethanol said bioreactor including: a culture system that cultures bacteria according to any one of embodiments 1-101 under conditions in which said bacteria partially or fully degrade cellulose in said lignocellulosic biomass to form one or more fermentable sugars; and a culture system that ferments said sugars to form a biofuel.

### Embodiment 110

A method of identifying cellulolytic enzyme combinations that enhance degradation of a particular substrate said method including: providing a plurality of recombinant bacteria according to any one of embodiments 1-101, wherein said bacteria each display at least two cellulolytic enzymes and different bacteria display different enzymes; contacting said substrate with said bacteria; and selecting bacteria that show enhanced degradation of said substrate and/or improved growth on said substrate.

### Embodiment 111

A method of identifying cellulolytic enzyme variants that enhance degradation of a particular substrate said method including: providing a plurality of recombinant bacteria according to any one of embodiments 1-101, wherein said bacteria each display at least one cellulolytic enzyme variant and different bacteria display different cellulolytic enzyme variants; contacting said substrate with said bacteria; and selecting bacteria that show enhanced degradation of said substrate and/or improved growth on said substrate.

### Embodiment 112

The method according to any one of embodiments 110 to 111, wherein said cellulolytic enzyme(s) and/or said cellulolytic enzyme variants comprise a mutant cellulolytic enzyme.

### Embodiment 113

The method of embodiment 112, wherein said mutant cellulolytic enzyme includes a mutant cellulase.

### Embodiment 114

The method according to any one of embodiments 110 to 113, wherein said selecting includes selecting bacteria that show improved growth on said substrate.

### DEFINITIONS

The term “nucleic acid” refers to a nucleotide polymer, and unless otherwise limited, includes known analogs of natural nucleotides that can function in a similar manner (e.g., hybridize) to naturally occurring nucleotides. The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; and RNA. The term nucleic acid encompasses double- or triple-stranded nucleic acid, as well as single-stranded molecules. In double- or triple-stranded nucleic acids, the nucleic acid strands need not be coextensive (i.e., a double-stranded nucleic acid need not be double-stranded along the entire length of both strands). The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

The terms “isolated”, when referring to an isolated nucleic acid or nucleic acid construct refers to a nucleic acid that either does not exist normally in nature, and/or that is constructed using for example, recombinant DNA techniques, and/or that is removed from nucleic acid sequences that would normally flank it in vivo, and/or that is removed from a cellular milieu. Isolated nucleic acids also include nucleic acids derived from the foregoing isolated nucleic acids, e.g., by propagation of a construct/vector/organism/virus/or microorganism containing such nucleic acid sequences.

“Operably linked” means that a gene (or other sequence to be expressed) and transcriptional regulatory sequence(s) are connected in such a way as to permit expression of the gene under control of the regulatory sequence(s).

“Exogenous” means a nucleic acid sequence that has been inserted into a host cell or a nucleic acid sequence or amino acid sequence derived from a nucleic acid sequence that has been inserted into a host cell. This includes introduced (inserted) nucleic acids that remain into the cytoplasm and introduced nucleic acids that integrate into the host cell genome (e.g., plasmids inserted into the host genome) as well as nucleic acid sequences and/or amino acids sequences derived from such. In certain embodiments an exogenous sequence can result from the cloning of a native gene from a host cell and the reinsertion of that sequence back into the host cell. In most instances, exogenous sequences are sequences that are derived synthetically, or from cells that are distinct from the host cell.

The terms “host cells” and “recombinant host cells” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “cellulolytic enzyme” refers to an enzyme that can participate in the degradation of cellulose or a cellulosic biomass.

The term “cellulosic biomass” refers to plant, algal, or other biomass that contains cellulose.

Lignocellulosic biomass refers to plant biomass that typically contains cellulose, hemicellulose, and lignin. The carbohydrate polymers (cellulose and hemicelluloses) are often tightly bound to the lignin. Lignocellulosic biomass can be grouped into four main categories: (1) agricultural residues (including corn stover and sugarcane bagasse), (2) dedicated energy crops, (3) wood residues (including sawmill and paper mill discards), and (4) municipal paper waste. Illustrative lignocellulosic biomass sources include, but are not limited to grasses, rice hulls, bagasse, jute, hemp, flax, bamboo, sisal, abaca, straw, corn cobs, corn stover, alfalfa, hay, coconut hair, seaweed, algae, and the like.

A cellulase is an enzyme that breaks down cellulose, especially in the wall structures, and a “cellulosome” is an array, cluster, or sequence of enzymes or cellulases that degrades cellulose. In various embodiments cellulosomes comprise catalytic subunits such as glycoside hydrolases, polysaccharide lyases and carboxyl esterases bound together by scaffoldins consisting of cohesins (cohesin domains) connected to other functional units such as the enzymes and carbohydrate binding modules via dockerins.

A “cohesin” or “cohesin domain” refers to a protein domain that interacts with a complementary domain, termed a “dockerin” or “dockerin domain”. Cohesin-dockerin interactions mediate the formation of cellulosome, or minicellulosomes.

The terms “linking dockerin” and “linking cohesin” refers to cohesins (cohesin domains) and dockerins (dockerin domains) that joint two backbone proteins (e.g., scaffoldins) to each other through a cohesin/dockerin interaction. in certain embodiments the linking dockerin is a type II dockerin and the linking cohesin is a type II cohesin.

A “protein encoding one or more cellulolytic enzymes” or a “protein comprising one or more cellulolytic” refers to a protein at least a portion of which displays cellulolytic activity. In certain embodiments the protein comprises a single cellulolytic enzyme and substantially the entire protein (absent processing and/or signaling sequences) comprises a single enzyme (e.g., a cellulase). In certain embodiments the protein comprises multiple (e.g., 2, 3, 4, 5, 6, or more) cellulolytic enzymes and in such instances each enzyme comprises a different “domain” in said protein. Similarly a protein comprising or encoding multiple cohesins refers to a protein comprising one or more domains each of which has the amino acid sequence of a cohesin, and in certain embodiments, is capable of binding to a corresponding dockerin.

When a Markush Group is described in the specification and/or claims it is intended that in various additional or alternative any subset of that Markush group is contemplated. Thus, for example, a Markush group consisting of elements A, B, and C also comprises a disclosure of a Markush Group consisting of A, and B, a Markush Group consisting of B, and C, and a Markush Group consisting of A and C as well as elements A, B, and C individually.

Where a range of values is provided, it is understood that each intervening value between the upper and lower limit of that range and any other stated or intervening value in that stated range, is contemplated. The upper and lower limits of these smaller ranges may independently be included in the smaller ranges, and are also contemplated, subject to any specifically excluded limit in the stated range. Where the stated range includes one or both of the limits, ranges excluding either or both of those included limits are also contemplated.

### BRIEF DESCRIPTION OF THE DRAWINGS

FIGS. 1A and 1B show that B. subtilis display minicellulosomes that assemble on the cell surface. FIG. 1A: Schematic of the B. subtilis minicellulosome. The scaffoldin protein (Scaf) contains type I cohesin modules from C. thermocellum (t), C. cellulolyticum (c) and R. flavefaciens (f), a family 3 carbohydrate binding module (CBM), and a cell wall sorting signal (CWSS) that enables it to be anchored to the cell wall. All enzymes are derived from C. cellulolyticum. These include the family 9 glycoside hydrolase (GH) enzyme fused to the R. flavefaciens type I dockerin module (Cel9E), the family 48 GH enzyme fused to the C. thermocellum type I dockerin module (Cel48F), and the family 5 GH enzyme fused to the C. cellulolyticum type I dockerin module (Cel5A). FIG. 1B: Immunoblots of the cell fractions demonstrating assembly of minicellulosomes containing one, two or three distinct cellulases. Cell wall (CW) and secreted protein (Sec) fractions were isolated from minicellulosome displaying cells (three cellulases, strain TDA17; two cellulases (Cel9E and Cel48F), TDA14; one cellulase ((Cel5A), TDA11) and cells that could only secrete the enzyme because the sortase and scaffoldin were not present (strain TDA18). Data for the Cel9E, Cel48F and Cel5A fusion enzymes are shown.

FIG. 2, panels A and B, show quantification and saturation-level determination of surface displayed Scaf protein. Panel A: Immunoblots of the cell wall fraction demonstrating that the Scaf proteins displayed on the cell-surface are entirely bound by cellulases. Cells of strain TDA17 were induced for SrtA, Scaf and cellulase expression (Expressed). The cells were then collected and incubated with excess amounts of E. coli purified Cel5A, Cel9E and Cel48F. Addition of purified enzymes (Purified) did not increase the intensity of the bands corresponding to Cel5A, Cel9E and Cel48F. Negative control strains TDA18 and TDA19 were unable to bind any cellulases while positive control strain TDA10 demonstrates the ability of E. coli purified enzymes to bind to cell-displayed Scaf. Panel B: Cel5A-associated activity on CMC was used to determine the amount of Scaf displayed per cell. TDA10 cells were induced to display covalently anchored Scaf (solid squares), followed by incubation with increasing amounts of E. coli purified Cel5A. After a washing step, the cell-associated Cel5A activity was measured; each cell is capable of displaying ˜150,000 Scaf molecules. Negative control strain TDA19 which is unable to successfully display Scaf (open squares) had negligible Cel5A-associated activity.

FIG. 3, panels A-C, show that B. subtilis displaying minicellulosomes grow on dilute acid pretreated biomass. Panel A: Growth of minicellulosome displaying B. subtilis on dilute acid pretreated corn stover (TDA17, open squares). Cells grew to similar densities as those cultured in the presence of glucose (solid squares, culture in glucose). Growth on biomass requires that the minicellulosome be attached to the peptidoglycan as cells lacking SrtA and Scaf failed to grow even though the cellulase enzymes are produced and secreted (strain TDA18, solid diamonds). Wild-type BAL2238 cells also failed to grow on biomass (data not shown). Panel B: Colony forming unit (CFU) measurements of cells grown on biomass and glucose. Symbols and growth conditions are as described in panel A. CFU/ml measurements are reported. Panel C: Growth of strains of B. subtilis displaying two cellulases (strain TDA14, open triangles; strain TDA15, grey triangles; strain TDA16, solid triangles) or one cellulase (strain TDA13, open circles; strain TDA12, grey circles; strain TDA11, solid circles) on dilute acid pretreated corn stover. Cells had a noticeable lag phase and were unable to reach cell densities similar to cells cultured in the presence of glucose (solid squares).

FIG. 4, panels A and B, show that cell displaying minicellulosomes efficiently hydrolyze dilute acid pretreated biomass. Panel A: Amount of insoluble dilute-acid treated corn stover remaining after incubation with minicellulosome displaying azide treated cells (strain TDA17, open boxes). Strain TDA18 which only secretes the three cellulases was unable to degrade biomass (solid diamonds). In this procedure, after incubation with azide treated cells, the insoluble residual biomass was washed to remove bound cells, dried and weighed. Panel B: Amount of insoluble dilute-acid pretreated corn stover remaining after incubation with strains of B. subtilis displaying one cellulase (strain TDA11, solid circles; strain TDA12, grey circles; strain TDA13, open circles) or two cellulases (strain TDA14, open triangles; strain TDA15, grey triangles, strain TDA16, solid triangles).

FIG. 5, panels A-C, show that cells displaying minicellulosomes containing three enzymes efficiently release soluble glycan and xylan from pretreated biomass and are comparable to commercially available cellulase cocktails. Panel A: Soluble reducing sugars released from dilute acid pretreated corn stover by cells displaying minicellulosomes (strain TDA17, open squares), cells that only secrete the enzymes (strain TDA18, solid diamonds), and the results of applying a Ctec2/Htec2 cellulase enzyme mixture to the biomass that is produced by Novozymes Inc. (crosses). Checkered squares represent data from cells displaying minicellulosomes that were supplemented with β-glucosidase. Panel B: Data is identical to that shown in panel A, but records the concentration of soluble glucose released. Panel C: Data is identical to that shown in panels A, but records the concentration of soluble xylose released.

FIG. 6, panels A-D, show that cells displaying minicellulosomes containing three enzymes grow on untreated corn stover, straw and switchgrass. Panel A: Growth curves of minicellulosome displaying B. subtilis cultured with untreated corn stover (open boxes), straw (solid squares) and switchgrass (grey squares). In each assay cells were grown on M9 salts and 0.5% w/v untreated biomass. Strain TDA18 which only secretes the enzymes could not grow on corn stover (solid diamonds), straw (grey diamonds) or switchgrass (open diamonds). Panel B: Reducing sugars released by minicellulosome displaying azide treated cells (strain TDA17). Sugars released from corn stover (open squares), switchgrass (grey squares) and straw (solid squares) is shown. Solid diamonds are data from control strain TDA18 cultured with untreated corn stover which produced only small amounts of soluble sugar (similar data not shown were obtained with straw and switchgrass). Panel C: Data is identical to that shown in panel B, but reports the concentration of soluble glucose released from untreated biomass. Panel D: Data is identical to that of panels B and C, but reports the concentration of soluble xylose released.

FIGS. 7A and 7B show that B. subtilis displays designer cellulosomes containing six cellulase enzymes that can self-assemble on the surface. FIG. 7A) Schematic of six-enzyme containing cellulosome displayed on the surface. The anchoring scaffoldin (Scaf-I) contains three type I cohesin modules derived from C. cellulolyticum (a), C. thermocellum (b) and R. flavefaciens (e); a carbohydrate binding module (CBM) derived from C. thermocellum CipA; a type-II cohesin module (C) from C. thermocellum; and a cell wall sorting signal (CWSS) enabling it to be anchored to the cell wall. Scaf-II contains the same three cohesin modules found in Scaf-I, and a type-II dockerin module (D) from C. thermocellum. The enzymes Cel5A, Cel9E, and Cel48 are derived from C. cellulolyticum and contain type-I dockerin modules from C. cellulolyticum, R. flavefaciens, and C. thermocellum, respectively. CelS, XynA and Man5A are derived from C. thermocellum and contain type-I dockerin modules from C. thermocellum, C. cellulolyticum, and R. flavefaciens, respectively. FIG. 7B) Immunoblots of cell wall fractions of strain TDA20 that can only secrete the enzymes (−SrtA) or those from strain TDA22 that can form a functional cellulosome containing six enzymes (+SrtA) on the cell surface.

FIG. 8 illustrates the quantification of the number of cell-surface displayed cellulosomes. Cel5A-associated activity on carboxymethyl cellulose was used to determine the amounts of cellulosomes that can be displayed per cell. B. subtilis cells displaying Scaf (strain TDA17 from Anderson et al. (2013) Applied and Environmental Microbiol., 79: 867-876) (diamonds), or Scaf-I and Scaf-II (strain TDA21, squares) were incubated with increasing amounts of purified Cel5A. Following incubation, the cells were washed, and incubated with carboxymethyl cellulose. After 1 hour, the amount of reducing sugars released was quantified. Cells displaying either Scaf or Scaf-I/Scaf-II are capable of displaying ˜150,000 cellulosomes per cell. Cells expressing the scaffoldin proteins but not SrtA (TDA19) (triangles) served as a negative control.

FIG. 9, panels A-D, shows that B. subtilis displaying three or six cellulase enzymes grows on dilute-acid pretreated lignocellulose. Panel A) Growth of B. subtilis displaying minicellulosomes containing enzymes Cel5A, Cel9E, and Cel48F (strain TDA17, diamonds), or enzymes Cel5A, Cel9E, Cel48F, CelS, Man5A, and XynA (strain TDA22, triangles) on dilute-acid pretreated corn stover. Cells that were unable to anchor the enzymes (squares) served as a negative control, and failed to grow during the time of the assay. A positive control, in which wild-type BAL2238 cells cultured with glucose (diamonds), was used. Panel B) Soluble reducing sugars released by B. subtilis displaying minicellulosomes containing either Cel5A, Cel9E, and Cel48F (strain TDA17, diamonds) or Cel5A, Cel9E, Cel48F, CelS, Man5A, and XynA (strain TDA22, triangles) and cells that only secrete the enzymes (strain TDA20, squares). The results of soluble reducing sugars released by the Novozyme cocktail Ctec2/Htec2 are also presented (black circles). Panel C) Data are identical as those presented in panel B, but show soluble xylose released. Panel D) Data are identical as those presented in panel B, but show soluble glucose released.

FIG. 10, panels A-D, shows that B. subtilis displaying minicellulosomes can grow efficiently on untreated corn stover. Panel A) B. subtilis displaying Cel5A, Cel9E, Cel48F, CelS, Man5A and XynA (strain TDA22, diamonds) display more efficient growth on untreated corn stover as the sole source of carbon than cells displaying Cel5A, Cel9E and Cel48F (strain TDA17, diamonds). B. subtilis growth with glucose (squares) and cells only able to secreted cellulase enzymes (squares) served as positive and negative controls, respectively. Panel B) Reducing sugars released by cells displaying six enzymes (strain TDA22, blue diamonds), three enzymes (strain TDA17, diamonds) and the Novozyme Ctec2/Htec2 cocktail (circles) from untreated corn stover. Cells unable to anchor cellulase enzymes (strain TDA20, squares) served as a negative control. Panel C) Data are identical as in panel B, but report soluble xylose released by the cells and cellulase cocktail. Panel D) Data are identical as in panel B, but report soluble glucose released by the cell and cellulase cocktail.

FIG. 11, panels A-D, shows that B. subtilis displaying minicellulosomes containing six enzymes can grow efficiently on untreated wheat straw. Panel A) B. subtilis displaying Cel5A, Cel9E, Cel48F, CelS, Man5A and XynA (strain TDA22, diamonds) more efficiently grow on untreated wheat straw than cells displaying only Cel5A, Cel9E and Cel48F (strain TDA17, diamonds). B. subtilis growth with glucose (squares) and cells induced to secrete cellulase enzymes (squares) served as positive and negative controls, respectively. Panel B) Reducing sugars released by cells displaying six enzymes (strain TDA22, diamonds), three enzymes (strain TDA17, diamonds) and the Novozyme Ctec2/Htec2 cocktail (circles) from untreated corn stover. Cells unable to anchor cellulase enzymes (strain TDA20, squares) served as a negative control. Panel C) Data are identical as in panel B, but report soluble xylose released by the cells and cellulase cocktail. Panel D) Data are identical as in panel B, but report soluble glucose released by the cell and cellulase cocktail.

FIG. 12, panels A-D, shows that B. subtilis displaying minicellulosomes containing six enzymes can grow efficiently on untreated switchgrass. Panel A) B. subtilis displaying Cel5A, Cel9E, Cel48F, CelS, Man5A and XynA (strain TDA22, diamonds) display more efficient growth characteristics on untreated switchgrass than cells displaying Cel5A, Cel9E and Cel48F (strain TDA17, diamonds). B. subtilis growth with glucose (squares) and cells that secreted cellulase enzymes (squares) served as positive and negative controls, respectively. Panel B) Reducing sugars released by cells displaying six enzymes (strain TDA22, diamonds), three enzymes (strain TDA17, diamonds) and the Novozyme Ctec2/Htec2 cocktail (black circles) from untreated corn stover. Cells unable to anchor cellulase enzymes (strain TDA20, squares) served as a negative control. Panel C) Data are identical as in panel B, but report soluble xylose released by the cells and cellulase cocktail. Panel D) Data are identical as in panel B, but report soluble glucose released by the cell and cellulase cocktail.

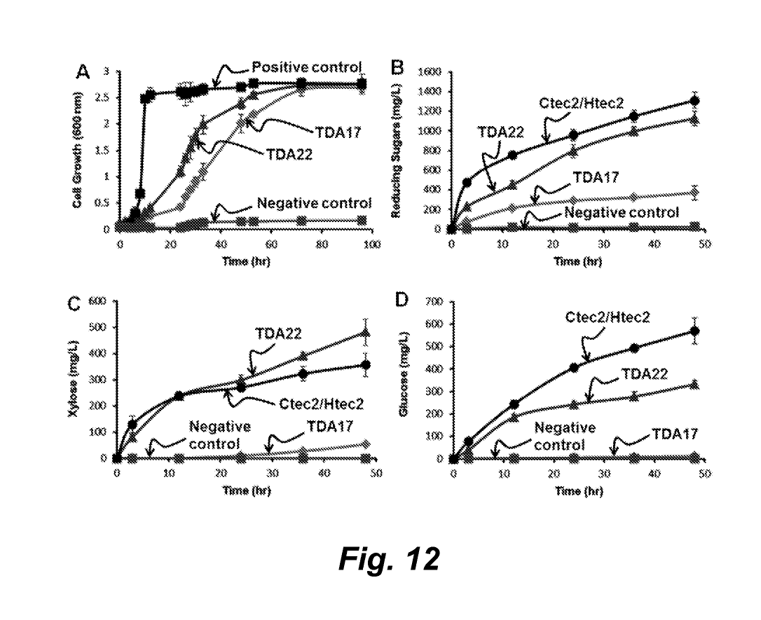

### DETAILED DESCRIPTION

It is desirable to produce biofuels and other bio-based chemicals and materials from renewable plant biomass (lignocellulose). One promising strategy is to create microbes that are consolidated bioprocessors (CBP). These microorganisms will breakdown biomass into its component sugars and then convert the degradation products into desired chemicals. At present, only a few CBP microbes have been developed and to the best of our knowledge none of them is widely used in industry. Bacillus subtilis is a promising CBP as it is already used in industry to produce a range of compounds (proteins, antibiotics and insecticides). Moreover, it has a robust genetic system, making it well suited for metabolic engineering which could enable it to produce other useful compounds. However, native strains of B. subtilis cannot efficiently degrade lignocellulose and use it as a nutrient to grow.

In various embodiments a protein display system is provided that enables multi-enzyme complexes to be self-assembled on the surface of B. subtilis. It is demonstrated that this new system can be used to create recombinant B. subtilis strains that can efficiently degrade lignocellulosic biomass. Furthermore, these cells can use biomass as a nutrient to grow. Additional modifications of the protein display system enable the number and types of enzymes displayed to be significantly increased to make even more potent cellulolytic organisms. The protein system can also be readily ported to other Gram-positive microbes which will enable them be used as a CBP.

This work has several potentially useful applications. Using the protein display system described herein: (1) the cellulolytic B. subtilis cells can be further engineered to develop a CBP that produces biocommodities such as ethanol from biomass, (2) It can be used to create highly cellulolytic B. subtilis cells that can replace more costly enzyme cocktails that are currently being used in industry to degrade biomass, and (3) The system we have developed can readily be ported to other Gram-positive bacterial species so as to enable them to use lignocellulose biomass as a nutrient.

Unlike most other recombinant cellulosome or minicellulosome systems, in the systems/organisms described herein all of the components of the minicellulosome are expressed in the microbe which effectively self-assembles the cellulosome. In addition, the recombinant organisms described herein display cellulase enzymes that are better suited for degrading lignocellulosic biomass (e.g., switchgrass, straw, corn stover, and the like).

It is believed the constructs described herein represent the first example of a self-assembling type-1 minicellulosome on the surface of B. subtilis. We have demonstrated that B. subtilis cells displaying the minicellulosome can efficiently degrade untreated biomass (corn stover, switchgrass, and straw). This is beneficial in that it potentially avoids costly pretreatment of the biomass (e.g. acid treatment) that is currently being used in industry. To the best of our knowledge, no recombinant microbe has ever been shown to be capable of growing on untreated biomass. Other microbes have been engineered to have cellulolytic activity, but they have only been shown to degrade purified cellulose substrates, such as phosphoric acid swollen cellulose and regenerated amorphous cellulose.

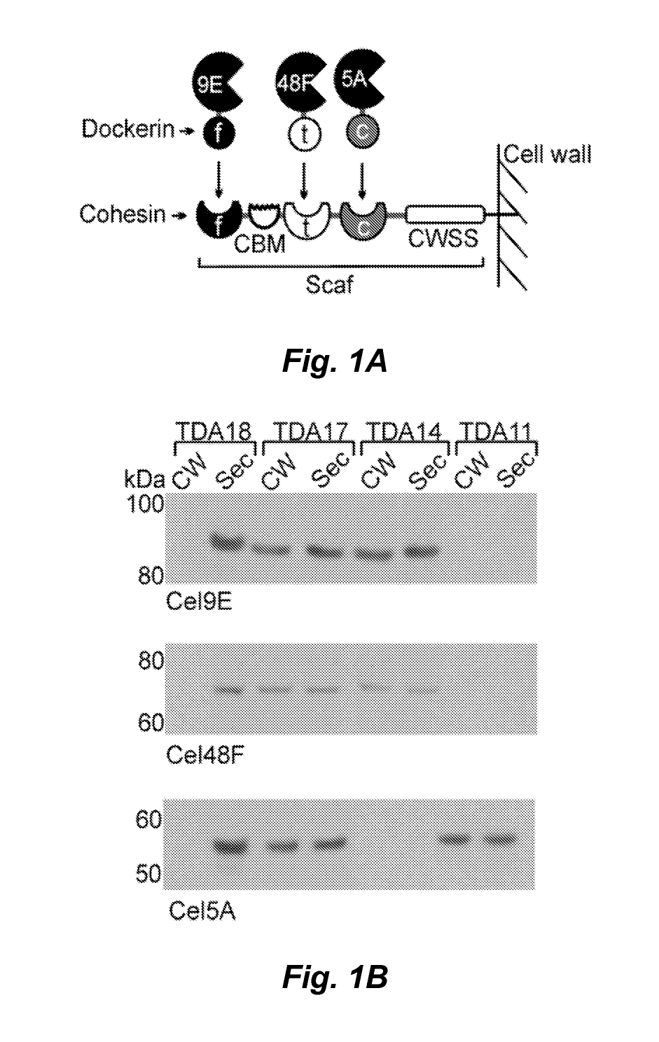

An illustrative schematic of one embodiment of the minicellulosome is shown in FIG. 1A, and described in detail herein in Example 1. Briefly, Strain TDA17 was generated to co-express five proteins: the SrtA sortase from B. anthracis, a chimeric scaffoldin (Scaf) composed of three cohesin modules that is covalently attached to the cell wall by SrtA, and three dockerin-cellulase fusion proteins that bind to the scaffoldin non-covalently via species-specific dockerin-cohesin interactions (Table 6). The three cellulases were derived from C. cellulolyticum and have complementary cellulose degrading activities: Cel5A (endoglucanase/xylanase, family 5 glycoside hydrolase (GH)), Cel48F (processive endoglucanase, family 48 GH), and Cel9E (exoglucanase, family 9 GH). Each protein component of the minicellulosome also contains an N-terminal signal sequence enabling them to be exported to the cell surface. The Scaf protein contains cohesin modules derived from C. cellulolyticum, C. thermocellum, and Ruminococcus flavefaciens, which selectively bind to their cognate dockerin modules fused to Cel5A, Cel48F, and Cel9E, respectively (FIG. 1A and Table 6). Scaf and the Cel9E enzyme also contain family 3 and 4 carbohydrate binding modules (CBM), respectively, which tether the enzyme complex to the cellulose component of the biomass. The scaf and srtA genes are integrated into the thrC locus of the chromosome, while genes expressing the three cellulase-dockerin fusion proteins are expressed from the pHCMC05-based plasmid pCellulase. All genes are expressed from a Pspac promoter and are IPTG inducible.

In fact, it has recently been noted that only twelve biomass-derived building blocks are needed to produce a range of commercial products. We have shown that our protein display system can be used to create B. subtilis strains that can grow on plant biomass. It is believed that cells or variant thereof can be used to produce these biocommodities or building-blocks from cheap plant biomass. An immediate application is to introduce the cellulolytic system described herein into existing organisms that are already used industrially to produce commercial products. This would enable the products to be produced from biomass and could significantly reduce costs.

Another application of the system described herein is to use the biomass-degrading cells as a replacement for enzyme cocktails that are currently used in industry to degrade biomass. The cells can be produced more cheaply than the enzymes and thereby reduce the costs associated with degrading biomass into its component sugars. An immediate application is lignocellulose degradation needed to produce lignocellulosic ethanol. Cells displaying the minicellulosomes would degrade the biomass into component sugars, which are then used in downstream ethanol producing fermentation reactions performed by S cerevisiae.

The system we have developed could also be used to engineer other species of Gram-positive bacteria to convert them into CPB or microbes that are dedicated to degrading biomass into sugars. The working prototype described herein has been demonstrated to successfully degrade lignocellulosic biomass into monosaccharides and oligosaccharides. It has also been demonstrated to grow on lignocellulose as the sole carbon source, indicating that these strains are well suited for use in a consolidated bioprocessor.

### Cell Wall Sorting Signal

As explained herein, in various embodiments, a sortase transpeptidase (e.g., Sortase A or analogues, homologues, or orthologues thereof) is exploited to couple a protein (e.g., a “scaffoldin” protein comprising one or a plurality of cohesin domains) to the surface (e.g., cell wall) of a Gram-positive microorganism. To facilitate this, the peptide can be provided with a cell wall sorting signal sequence that is recognized by the sortase transpeptidase.

The examples provided herein use a C-terminal portion of the Staphylococcus aureus Fibronectin Binding Protein B, which contains a 123 amino acid spacer segment and the cell wall sorting signal (CWS). This S. aureus CWS sequence is identical to CWS found in many B. anthracis surface proteins that are anchored to the cell wall of B. anthracis by SrtA

Typically cell wall sorting signals comprise an LPXTG (SEQ ID NO: 1) motif (where X is any amino acid), a C-terminal hydrophobic domain and a charged tail. Homologous sequences are found in many surface proteins of Gram-positive bacteria (see, e.g., Schneewind et al. (1993) EMBO J., 12(12): 4803-4811, which describes a number of cell wall sorting signals, illustrated below in Table 1).

TABLE 1
Illustrative cell wall sorting signals in
surface proteins of Gram-positive bacteria.

| | | SEQ | |
|---|---|---|---|
| Protein | Cell Wall Sorting Signal (CWSS) | ID NO | Bacterial Species |
| SPA | LPETGEENPFIGTTVFGGLSLALGAALLAGRRREL | 2 | S. aureus |
| FNBP | LPETGGEESTNKGMLFGGLFSILGLALLRRNKKNHKA | 3 | S. aureus |
| SPAA | LPATGDSSNAYLPLLGLVSLTAGFSLLGLRRKQD | 4 | S. sobrinus |
| PRGB | LPKTGEKQNVLLTVVGSLAAMLGLAGLGFKRRKETK | 5 | E. faecalis |
| TEE | LPSTGSIGTYLFKAIGSAAMIGAIGIYIVKRRKA | 6 | S. pyogenes |
| INLA | LPTTGDSDNALYLLLGLLAVGTAMALTKKARASK | 7 | L. monocytogenes |
| FIM. | LPLTGANGVIFLTIAGALLVAGGAVVAYANKRRHVAKH | 8 | A. viscosus |
| BAC | LPYTGVASNLVLEIMGLLGLIGTSFIAMKRRKS | 9 | S. agalactiae |
| CNA | LPKTGMKIITSWITWVFIGILGLYLILRKRFNS | 10 | S. aureus |
| WAP | LPSTGEQAGLLLTTVGLVIVAVAGVYFYRTRR | 11 | S. mutans |
| EMM | LPSTGETANPFFTAAALTVMATAGVAAVVKRKEEN | 12 | S. pyogenes |

These cell wall sorting signals are intended to be illustrative and not limiting. Using the teachings provided herein, numerous other cell wall sorting signals can be incorporated in the expression/display systems described herein.

While in certain embodiments, cell wall sorting signals comprising the LPXTG motif are preferred, they need not be limited to this motif. Based on homology sortases thus far identified are typically grouped into four or five subgroups or classes (see, Table 2). Each subgroup, in addition to distinctions in sequence, can be distinguished from one another based on membrane topology, genome position, and preference for substrates with specific amino acids within the cell wall sorting signal pentapeptide motif (Comfort and Clubb (2004) Infect. Immun., 72: 2710-2722; Dramsi et al. (2005) Res. Microbiol. 156: 289-297). As indicated above, the prototypical sortase is sortase A, first identified in S. aureus. Sortase A appears to anchor a large number and broad range of surface proteins. The sortase A subgroup of enzymes also seems to share a preference for the LPXTG (SEQ ID NO: 13) cell wall sorting signal motif. The second subgroup of enzymes, sortase B, along with its substrate (IsdC in S. aureus), is encoded in an iron transport operon involved in heme-iron uptake. Enzymes belonging to the sortase B subgroup contain three amino acid segments not found in sortase A and recognize substrates containing an NPQTN (SEQ ID NO:14) motif rather than the canonical LPXTG (SEQ ID NO: 15). The third class, designated sortase C or subfamily 3, contains a C-terminal hydrophobic domain (Id.). Subfamily 3 enzymes also share a preference for substrates containing the LPXTG cell wall sorting signal motif, often followed by a second G residue (i.e., LPXTGG, (SEQ ID NO:16). A fourth subgroup can be defined after alignment of sortase sequences. It has been noted as the sortase D subgroup (Dramsi et al. (2005) Res. Microbiol. 156: 289-297) or subfamilies 4 and 5, as sortases in this subgroup can be distinguished based on the cell wall sorting signals of their associated substrates (Comfort and Clubb (2004) Infect. Immun., 72: 2710-2722). Sortases belonging to subfamily 4 are predicted to anchor proteins bearing the unique LPXTA(ST) (SEQ ID NO:17) motif (Id.). An alanine residue in the last position of the substrate motif suggests that the subfamily 4 enzymes fulfill a nonredundant role within the cell (Id.). Many high-G/C bacteria contain sortases belonging to subfamily 5, and most do not harbor sortase A. This subgroup of sortase enzymes shares substrate specificity for proteins containing an LAXTG (SEQ ID NO:18) motif (Id.).

TABLE 2
Sortase classifications.

| | | Membrane | |
|---|---|---|---|
| Sortase class | Cleavage | anchor | |
| (subfamily)a | site^{b} | domain^{c} | Bacterial taxa^{d} |
| A (1) | LPkT-Ge* | N terminus | Bacillus, Listeria, |
| | | | Staphylococcus, Enterococcus, |
| | | | Lactobacillaceae, |
| | | | Streptococcaceae |
| B (2) | NPqt-nd* | N terminus | Bacillus, Listeria, |
| | | | Staphylococcus, |
| | | | Streptococcaceae, Clostridia |
| C (3) | 1PkT-GG | C terminus | Actinobacteria, Bacillus, |
| | | | Enterococcus, |
| | | | Leuconostocaceae, |
| | | | Streptococcaceae, Clostridia |
| D (4) | LPnT-At | N terminus | Bacillus |
| D (5) | LAeT-Ga | N terminus | Actinobacteria |

aSortase subfamily and class assignments are based on sequence, membrane topology, genomic positioning, and preference for specific amino acids within the cell wall sorting signal pentapeptide motif region of their cognate substrates.
^{b}Cell wall sorting signal pentapeptide motif. Uppercase letters represent amino acids that are absolutely conserved. Asterisks indicate that the cleavage site has been verified experimentally.

Accordingly in various embodiments, display systems that utilize any of these cell wall sorting sequences are contemplated for use in the methods and constructs described herein.

### Cohesin and Dockerin Proteins/Protein Domains.

As described herein, in various embodiments, the display system(s) utilize more proteins (e.g., scaffoldins) comprising one or more, preferably two or more, cohesin domains (e.g., cohesin I domains) that interact with dockerin domains to anchor and/or organize one or more enzymes on the surface of the Gram-positive bacterium.

In various embodiments the systems contemplated herein can comprise one or more dockerin domains selected from the group consisting of a dockerin I domain, a dockerin II domain, and a dockerin III domains. Correspondingly, in various embodiments the systems contemplated herein can comprise one or more cohesin domains selected from the group consisting of a cohesin I domain, a cohesin II domain, and a cohesin III domains that binds to its corresponding dockerin sequence. In certain embodiments the dockerin and/or cohesin domains comprise a domain derived from Clostridium thermocellum.

The sequences of cohesins and dockerins are well known to those of skill in the art (see, e.g., Ding et al. (2003) Genet. Eng. (N Y) 25: 209-225 for cellulosome cohesins and dockerins, and Peer et al. (2009) FEMS Microbiol Lett. 291(1): 1-16 for non-cellulosome cohesins and dockerins). In various embodiments specific cohesin-dockerin pairs are chosen so as to enable specific complexes to form on the cell surface even if multiple enzymes are present.

While the display system described herein is exemplified using cohesin-dockerin pairs, it will be recognized that, in certain embodiments, other protein-protein interaction pairs can be used as long as one member of the pair becomes covalently attached to the cell wall and the other is fused to the cellulolytic enzyme(s) so as to enable enzyme complex formation on the cell surface.

### Cellulolytic Enzymes and Minicellulosomes.

In various embodiments Gram-positive bacteria are engineered using the methods described herein to display one or more enzymes. In certain embodiments the enzymes are cellulolytic enzymes and/or other enzymes useful in the synthesis of biofuels from lignocellulosic biomass. In various embodiments it will be recognized that the “cellulases” can include, but are not limited to, the cellobiohydrolases, e.g., cellobiohydrolase I and cellobiohydrolase II, as well as the endoglucanases. In various embodiments “cellulolytic enzymes” include, but are not limited to, cellobiohydrolases, e.g. cellobiohydrolase I and cellobiohydrolase II, as well as endoglucanases and beta-glucosidases.

In various embodiments the digestion of cellulose and hemicellulose is facilitated by the use of several types of enzymes acting cooperatively. In certain embodiments at least three categories of enzymes are utilized to convert cellulose into fermentable sugars: endoglucanases that cut the cellulose chains at random; cellobiohydrolases that cleave cellobiosyl units from the cellulose chain ends and beta-glucosidases that convert cellobiose and soluble cellodextrins into glucose. Among these three categories of enzymes involved in the biodegradation of cellulose, cellobiohydrolases are useful for the degradation of native crystalline cellulose. Cellobiohydrolase I, also referred to as a cellulose 1,4-beta-cellobiosidase or an exoglucanase, exo-cellobiohydrolase or 1,4-beta-cellobiohydrolase catalyzes the hydrolysis of 1,4-beta-D-glucosidic linkages in cellulose and cellotetraose, by the release of cellobiose from the non-reducing ends of the chains. Cellobiohydrolase II activity is identical, except that cellobiohydrolase II attacks from the reducing ends of the chains.

In various embodiments the cellulolytic enzymes are organized into a cellulosome or minicellulosome (see, e.g., FIG. 1A). Cellulosome complexes are multi-enzyme complexes that can be designed for efficient degradation of plant cell wall polysaccharides, notably cellulose. Cellulosomes typically comprises a multifunction integrating scaffold (called scaffoldin), responsible for organizing the various cellulolytic subunits (e.g., the enzymes) into the complex. The scaffolidin comprises one or more cohesin domains. Enzymes attached to dockerins are organized on the scaffoldin by specific interactions between cohesins and dockerins that specifically or preferentially bind to particular cohesins. In certain embodiments attachment of the cellulosome to its substrate is mediated by a scaffoldin-borne cellulose-binding module (CBM) that can comprises part of the scaffoldin subunit.

The displayed cellulosomes can be simple cellulosome systems containing a single scaffoldin or complex cellulosome systems that exhibit multiple types of interacting scaffoldins. In various embodiments each scaffoldin can contain one, two, three, four, five, six, seven, eight, nine, or 10 or more cohesin domains. The arrangement of the modules on the scaffoldin subunit and the specificity of the cohesin(s) and/or dockerin for their modular counterpart determine the overall architecture of the cellulosome. Several different types of scaffoldins have been described and are useful in the construction of minicellulosomes according to the methods described herein. The primary scaffoldins incorporate the various dockerin-bearing subunits directly into the cellulosome complex, adaptor scaffoldins increase the repertoire or number of components into the complex, and the anchoring scaffoldins attach the complex to the bacterial cell surface.

Scaffoldins are well known to those of skill in the art and can readily be identified with a simple GenBank search for the term “scaffoldin”.

In certain embodiments the cellulolytic enzymes comprising the cellulosome or individually displayed on the surface of the bacteria comprise one or more enzymes collected from the group consisting of an exoglucanase, an endoglucanase, a glycosyl hydrolase, a cellulase, a hemicellulase, a xylanase, a cellobiohydrolase, a beta-glucosidase, a mannanse, a xylosidase (e.g., a β-xylosidase), an arabinofuranosidase, and/or a glucose oxidase. Illustrative, but non-limiting, enzymes suitable for display using the systems described herein are shown in Table 3.

TABLE 3
Illustrative enzymes suitable for display on Gram-positive
bacteria using the methods described herein.

| | Genbank Accession |
|---|---|
| Enzyme | No |
| Endoglucanases: | |
| Clostridium thermocellum endoglucanse CelG | 69390 |
| Clostridium thermocellum endoglucanse CelD | X04584 |
| Clostridium thermocellum endoglucanse CelQ | AB047845 |
| Clostridium thermocellum endoglucanse CelR | AJ585346 |
| Clostridium thermocellum endoglucanse CelN | AJ275974 |
| Exoglucanases: | |
| Clostridium thermocellum exoglucanase CelS | L06942 |
| Cellobiohydrolases: | |
| Clostridium thermocellum cellobiohydrolase CbhA | X80993 |
| Clostridium thermocellum cellobiohydrolase CelK | AF039030 |
| Clostridium thermocellum cellobiohydrolase CelO | AJ275975 |
| Xylanases: | |
| Clostridium thermocellum xylanase XynD | AJ585345 |
| Clostridium thermocellum xylanase XynC | D84188 |
| Clostridium thermocellum xylanase XynA | AB010958 |
| Hemicellulases: | |
| Clostridium thermocellum lichenase LicB | X63355 |
| Clostridium thermocellum chitinase ChiA | CAB52403 |
| Clostridium thermocellum mannase ManA | CAD12659 |

In addition, a large number of other suitable enzymes are described in U.S. Patent Publication 2010/0189706 which is incorporated herein by reference for any one or more of the cellulolytic enzymes described herein. Cellulosomes are also described by Fontes and Gilbert (2010) Annu. Rev. Biochem., 79: 655-681.

In certain embodiments the cellulosome that is to be displayed can be engineered based upon the cellulosic material to be metabolized. For example, different cellulases and other enzymes may be engineered into a cellulosome pathway depending upon the sources of substrate. Illustrative substrate sources include, but are not limited to, alfalfa, corn stover, crop residues, debarking waste, forage grasses, forest residues, municipal solid waste, paper mill residue, pomace, sawdust, spent grains, spent hops, switchgrass, and wood chips. Some substrate sources can have a larger percentage of cellulose compared to other source, which may have a larger percentage of hemicellulose.

A hemicellulose substrate typically comprises short, branched chains of sugars and can comprise a polymer of five different sugars. Hemicellulose comprises five-carbon sugars (e.g., D-xylose and L-arabinose) and six-carbon sugars (e.g., D-galactose, D-glucose, and D-mannose) and uronic acid. The sugars are typically substituted with acetic acid. Hemicellulose is relatively easy to hydrolyze to its constituent sugars. When hydrolyzed, the hemicellulose produces xylose (a five-carbon sugar) or six-carbon sugars from hardwoods or softwoods, respectively.

Proteins or polypeptides having the ability to convert the hemicellulose components into carbon sources that can be used as a substrate for biofuel production includes, for example, cellobiohydrolases (Accessions: AAC06139, AAR87745, EC 3.2.1.91, 3.2.1.150), cellulases (E.C. 3.2.1.58, 3.2.1.4, Accessions: BAA12070, BAB64431); chitinases (E.C. 3.2.1.14, 3.2.1.17, 3.2.1.-, 3.2.1.91, 3.2.1.8, Accessions: CAA93150, CAD12659), various endoglucanases (E.C. 3.2.1.4, Accessions: BAA92430, AAG45162, P04955, AAD39739), exoglucanases (E.C. 3.2.1.91, Accessions: AAA23226), lichenases (E.C. 3.2.1.73, Accessions: P29716), mannanases (E.C. 3.2.1.4, 3.2.1.-, Accessions: CAB52403), pectate lyases (E.C. 4.2.2.2, Accessions: AAG59609), xylanase (E.C. 3.2.1.136, 3.2.1.156, 3.2.1.8, Accessions: BAA33543, CAA31 109) and silase (E.C. 3.2.2.-, 2.7.7.7, Accessions: CQ80097S).

Cellulases are a class of enzymes produced chiefly by fungi, bacteria, and protozoans that catalyze the hydrolysis of cellulose. However, there are also cellulases produced by other types of organisms such as plants and animals. Several different kinds of cellulases are known, which differ structurally and mechanistically. The EC number for cellulase enzymes is E.C. 3.2.1.4. Assays for testing cellulase activity are known in the art.

Polypeptides having xylanase activity are also useful in synthetic cellulosomes. Xylanase is the name given to a class of enzymes which degrade the linear polysaccharide beta-1,4-xylan into xylose, thus breaking down hemicellulose. The EC number for xylanase enzymes is E.C. 3.2.1.136, 3.2.1.156, 3.2.1.8. Assays for testing xylanase activity are known in the art.

In certain embodiments the minicellulosome comprises at least two different, or at least three different, or at least four different, or at least five different, or at least six different, or at least seven different, or at least 8 different, or at least 9 different, or at least 10 different, or at least 11 different, or at least 12 different cellulolytic (or other degredative) enzymes. In certain embodiments the two enzymes comprise two different or three different, or four different, or five different, or six different enzymes selected from the group consisting of endocellulase/endocellulase, exocellulase/endocellulase, beta-glucosidase (cellobiase)/endocellulase, oxidative cellulase/endocellulase, xylanase/endocellulase, hemicellulase/endocellulase, lichenase/endocellulase, chitenase/endocellulase, xylanase/endocellulase, cellulose phosphorylase/endocellulase, endocellulase/exocellulase, exocellulase/exocellulase, beta-glucosidase (cellobiase)/exocellulase, oxidative cellulase/exocellulase, xylanase/exocellulase, hemicellulase/exocellulase, lichenase/exocellulase, chitenase/exocellulase, xylanase/exocellulase, cellulose phosphorylase/exocellulase, endocellulase/beta-glucosidase, exocellulase/beta-glucosidase, beta-glucosidase (cellobiase)/beta-glucosidase, oxidative cellulase/beta-glucosidase, xylanase/beta-glucosidase, hemicellulase/beta-glucosidase, lichenase/beta-glucosidase, chitenase/beta-glucosidase, xylanase/beta-glucosidase, cellulose phosphorylase/beta-glucosidase, endocellulase/oxidative cellulase, exocellulase/oxidative cellulase, beta-glucosidase (cellobiase)/oxidative cellulase, oxidative cellulase/oxidative cellulase, xylanase/oxidative cellulase, hemicellulase/oxidative cellulase, lichenase/oxidative cellulase, chitenase/oxidative cellulase, xylanase/oxidative cellulase, cellulose phosphorylase/oxidative cellulase, endocellulase/xylanase, exocellulase/xylanase, beta-glucosidase (cellobiase)/xylanase, oxidative cellulase/xylanase, xylanase/xylanase, hemicellulase/xylanase, lichenase/xylanase, chitenase/xylanase, xylanase/xylanase, cellulose phosphorylase/xylanase, endocellulase/hemicellulase, exocellulase/hemicellulase, beta-glucosidase (cellobiase)/hemicellulase, oxidative cellulase/hemicellulase, xylanase/hemicellulase, hemicellulase/hemicellulase, lichenase/hemicellulase, chitenase/hemicellulase, xylanase/hemicellulase, cellulose phosphorylase/hemicellulase, endocellulase/lichenase, exocellulase/lichenase, beta-glucosidase (cellobiase)/lichenase, oxidative cellulase/lichenase, xylanase/lichenase, hemicellulase/lichenase, lichenase/lichenase, chitenase/lichenase, xylanase/lichenase, cellulose phosphorylase/lichenase, endocellulase/chitenase, exocellulase/chitenase, beta-glucosidase (cellobiase)/chitenase, oxidative cellulase/chitenase, xylanase/chitenase, hemicellulase/chitenase, lichenase/chitenase, chitenase/chitenase, xylanase/chitenase, cellulose phosphorylase/chitenase, endocellulase/xylanase, exocellulase/xylanase, beta-glucosidase (cellobiase)/xylanase, oxidative cellulase/xylanase, xylanase/xylanase, hemicellulase/xylanase, lichenase/xylanase, chitenase/xylanase, xylanase/xylanase, cellulose phosphorylase/xylanase, endocellulase/cellulose phosphorylase, exocellulase/cellulose phosphorylase, beta-glucosidase (cellobiase)/cellulose phosphorylase, oxidative cellulase/cellulose phosphorylase, xylanase/cellulose phosphorylase, hemicellulase/cellulose phosphorylase, lichenase/cellulose phosphorylase, chitenase/cellulose phosphorylase, xylanase/cellulose phosphorylase, and cellulose phosphorylase/cellulose phosphorylase.

In certain embodiments the minicellulosome comprises at least three different cellulolytic (or other degredative) enzymes. In certain embodiments the three different enzymes comprise an enzyme pair selected from the group listed above, combined with one enzyme selected from the group consisting of an endocellulase, an exocellulase, a beta-glucosidase (cellobiase), an oxidative cellulase, a xylanase, a hemicellulase, a lichenase, a chitenase, a xylanase, and a cellulose phosphorylase.

It will be recognized that the enzymes, and enzyme combinations, identified above are intended to be illustrative and not limiting. Using the teachings provided herein, the display of numerous other enzymes will be available to one of skill in the art.

### Carbohydrate Binding Domain/Module (CBD/CBM)

In various embodiments, to facilitate interaction of displayed enzyme(s) with their substrate (e.g., cellulose) the displayed protein comprises a substrate binding domain (e.g., a carbohydrate binding domain). Suitable substrate binding domains include, but are not limited to, carbohydrate binding domains, cellulose binding domains, cellulose binding modules, or other binding domains.

The amino acid sequence of cellulose binding peptides and/or binding domains are well known to those of skill in the art. Carbohydrate binding peptides include peptides e.g., proteins and domains (portions) thereof, that are capable of, binding to a plant derived cellulosic (e.g., lignocellulosic) material. Carbohydrate binding peptides include, for example, peptides screened for their cellulose binding activity out of a library, as well as naturally occurring cellulose binding peptides or peptide domains.

The carbohydrate binding domain can include any amino acid sequence expressible in plants which binds to a cellulose polymer. For example, the cellulose binding domain or protein can be derived from a cellulase, a binding domain of a cellulose binding protein or a protein screened for, and isolated from, a peptide library, or a protein designed and engineered to be capable of binding to cellulose or to saccharide units thereof. The cellulose binding domain or protein can be naturally occurring or synthetic. Suitable polysaccharidases from which a carbohydrate binding domain can be obtained includes, but is not limited to a β-1,4-glucanase. In certain embodiments, a cellulose binding domain or protein from a cellulase or scaffoldin is used.

Carbohydrate binding domains/modules are well known to those of skill in the art (see, e.g., Tomme et al. (1995) in Enzymatic Degradation of Insoluble Polysaccharides (Saddler and Penner, eds.), Cellulose-binding domains: classification and properties. pp. 142-163, American Chemical Society, Washington). Cellulose binding domains are also described in U.S. Pat. No. 5,837,814 and in U.S. Patent publication 2011/0005697 which are incorporated herein by reference for the cellulose binding domains described therein. In particular, U.S. Patent Publication No: 2011/0005697 identifies proteins containing putative β-1,3-glucan-binding domains (see, e.g., Table 1 therein, Table 4 below); proteins containing Streptococcal glucan-binding repeats (Cp1 superfamily) (see e.g., Table 2 therein, Table 5 below), and the like.

TABLE 4
Proteins containing putative β-1,3 glucan binding domains.

| Source (strain) | Protein | Accession No. | Ref |
|---|---|---|---|
| Type I: | | | |
| B. circulans (WL-12) | GLCA1 | P23903/M34503/JQ0420 | 1 |
| B. circulans (IAM 1165) | BglH | JN0772/D17519/267033 | 2 |
| Type II: | | | |
| Actinomadura sp. (FC7) | XynII | U08894 | 3 |
| Arthrobacter sp. | GLCI | D23668 | 10 |
| (YCWD3) | | | |
| O. xanthineolytica | GLC | P22222/M60826/A39094 | 4 |
| R. faecitabidus | RPI | Q05308/A45053/D10753 | 5, 6 |
| (YLM-50) | | | |
| R. communis | Ricin | A12892 | 7 |
| S. ividans (1326) | XlnA | P26514/M64551/JS07986 | 8 |
| R. tridentatus | FactorGa | D16622 | 9 |

1. Yahata et al. Gene, 86: 113-117
2. Yamamoto et al. (1993) Biosci. Biotechnol. Biochem., 57: 1518-1525.
3. Harpin et al. (1994) EMBL Data Library.
4. Shen et al. (1991) J. Biol. Chem., 266: 1058-1063
5 Shimoi et al. (1992) J. Biol.Chem., 267: 25189-25195.
6. Shimoi et al. (1992) J. Biochem., 110: 608-613
7. Horn et al. (1989) Patent A12892
8. Shareck et al. (1991) Gene, 269: 1370-1374.
9. Seki et al. (1994) J. Biol. Chem., 269: 1370-1374
10. Watanabe et al. (1993) EMBL Data Library.

TABLE 5
Illustrative proteins containing Streptococcal glucan-binding repeats.

| Source | Protein | Accession No. | Ref. |
|---|---|---|---|
| S. downei (sobrinus) | GTF-I | D13858 | 1 |
| S. downei (sobrinus) | GTF-I | P11001/M17391 | 2 |
| S. downei (sobrinus) | GTF-S | P29336/M30943/A41483 | 3 |
| S. downei (sobrinus) | GTF-I | P27470/D90216/A38175 | 4 |
| S. downei (sobrinus) | DEI | L34406 | 5 |
| S. mutans (Ingbritt) | GBP | M30945/A37184 | 6 |
| S. mutans (GS-5) | GTF-B | A33128 | 7 |
| S. mutans (GS-5) | GTF-B | P08987/M17361/B33135 | 8 |
| S. mutans | GTF-B^{3′-ORF} | P05427/C33135 | 8 |
| S. mutans (GS-5) | GTF-C | P13470/M17361/M22054 | 9 |
| S. mutans (GS-5) | GTF-C | Not available | 10 |
| S. mutans (GS-5) | GTF-D | M29296/A45866 | 11 |
| S. salivarius | GTF-J | A44811/S22726/S28809/ | 12 |
| | | Z11873/M64111 | |
| S. salivarious | GTF-K | S22737/S22727/Z11872 | 13 |
| S. salivarious | GTF-L | L35495 | 14 |
| (ATCC25975) | | | |
| S. salivarious | GTF-M | L35928 | 14 |
| (ATCC25975) | | | |
| S. pneumoniae R6 | LytA | P06653/A25634/M13812 | 15 |
| S. pneumoniae | PspA | A41971/M74122 | 16 |
| Phage HB-3 | HBL | P06653/A25634/M13812 | 17 |
| Phage Cp-1 | CPL-1 | P15057/J03586/A31086 | 18 |
| Phage CP-9 | CPL-9 | P19386/M34780/JQ0438 | 19 |
| Phage EJ-1 | EJL | A42936 | 20 |
| C. difficile (VPI | ToxA | P16164/A37052/M30307/ | 21 |
| 10463) | | X51797/S08638 | |
| C. difficile (BARTS | ToxA | A60991/X17194 | 22 |
| W1) | | | |
| C. difficile (VPI | ToxB | P18177/X53138/X6098/ | 23, |
| 10463) | | S10317 | 24 |
| C. difficile (1470) | ToxB | X44271/Z23277 | 25, |
| | | | 26 |
| C. novyi | α-toxin | S44272/Z23280 | 27 |
| C. novyi | α-toxin | Z48636 | 28 |
| C. acetobutylicum | CspA | 549225/Z37723 | 29 |
| (NCIB8052) | | | |
| C. acetobutylicum | CspB | Z50008 | 30 |
| (NCIB8052) | | | |
| C. acetobutylicum | CspC | Z50033 | 30 |
| (NCIB8052) | | | |
| C. acetobutylicum | CspD | Z50009 | 30 |
| (NCIB8052) | | | |

References:
1. Sato et al. (1993) DNA sequence 4: 19-27
2. Ferreti et al. (1987) J. Bacteriol., 169: 4271-4278
3. Gilmore et al. (1990) J. Infect. Immun., 58: 2452-2458.
4. Abo et al. (1991) J. Bacteriol., 173: 989-996.
5. Sun et al. (1994) J. Bacteriol., 176: 7213-7222.
6. Banas et al. (1990) J. Infect. Immun., 58: 667-673.
7. Shiroza et al. (1990) Protein Sequence Database.
8. Shiroza et al. 91987) J. Bacteriol., 169: 4263-4270.
9. Ueda et al. (1988) Gene, 69: 101-109.
10. Russel (1990) Arch. Oral Biol., 35: 53-58.
11. Honda et al. (1990) J. Gen. Microbiol., 136: 2099-2105
12. Giffard et al. (1991) J. Gen. Microbiol., 137: 2577-2593.
13. Jacques (1992) EMBL Data Library
14. Simpson et al. (1995) J Infect. Immun., 63: 609-621
15. Garcia et al. (1986) Gene 43: 265-272.
16. Yother et al. (1992) J. Bacteriol., 374: 601-609
17. Romero et al. (1990) J. Bacteriol., 5064-5070.
18. Garcia et al. (1988) PNAS 85: 914-918
19. Garcia et al. (1990) Gene, 86: 81-88.
20. Diaz et al. (1992) J. Bacteriol., 174: 5516-5525.
21. Dove et al. (1990) J. Infect. Immun., 58: 480-488.
22. Wren et al. (1990) FEMS Microbiol. Lett., 70: 1-6.
23. Barroso et al. (1990) Nucl. Acids. Res., 18: 4004
24. von Eichel-Streiber et al. (1992) Mol. Gen. Genet., 233: 260-268.
25. Sartinger et al. (1993) EMBL Data Library.
26. von Eichel-Streiber et al. (1995) Mol Microbiol.
27. Hoffman et al. (1993) EMBL Data Library.
28. Hoffman et al. (1995) Mol. Gen.
29. Sanches et al. (1994) EMBL Data Library.
30. Sanches et al. (1995) EMBL Data Library.

In various embodiments the Kₐ. for binding of the carbohydrate binding domains/proteins to cellulose is at least in the range of weak antibody-antigen extractions, i.e., at least 10³ M⁻¹, preferably at least 10⁴ M⁻¹, most preferably at least 10⁶ M⁻¹.

### Secretory Signal Sequence.

In various embodiments the peptide comprising the cell wall sorting signal (CWS) also contains a secretory signal sequence to enhance/facilitate transport through the cell membrane. Typical Gram-positive secretory signal peptides are N-terminal peptides.

Gram-positive secretion signals are well known to those of skill in the art. In certain embodiments the secretory signal sequence comprises a B. subtilis phrC secretory signal or homologues thereof.

### Extended Scaffoldins for Attachment of Additional Enzymes

In various embodiments, the number and types of enzymes that can be displayed in each cellulosome (minicellulosome) can be increased by using multiple polypeptide fragments to construct a cell wall attached extended scaffoldin that in turn coordinates the binding of cellulases that are displayed on the cell's surface. This approach eliminates the need to express and display a single large scaffoldin polypeptide which can be problematic. The use of multiple polypeptide fragments to construct an “extended” scaffolidin provides a simple and effective way in which to expand the number of enzymes displayed on the surface of a Gram-positive bacterium (e.g., B. subtilis).

In certain illustrative, but non-limiting embodiments, the polypeptide fragments are expressed with “complementary” “linking scaffoldins and linking dockerins” that join the polypeptide fragment into an extended scaffoldin. Thus, for example a first polypeptide can be attached to the bacterial cell wall and bear a terminal “linking” (linker) cohesin or dockerin. This terminal linking cohesin or dockerin interacts with a corresponding cohesin or dockerin on a second polypeptide thereby providing an extended scaffoldin. Optional, the second polypeptide can bear a second linking cohesin or dockerin that interacts with a corresponding linker or dockerin on a third polypeptide fragment thereby facilitating the attachment of the third polypeptide to the second.

As proof of principle, the utility of this method is demonstrated herein in Example 2 by displaying a complex that contains six enzymes (instead of three enzymes). Cells containing the enlarged six enzyme complex have significantly improved cellulolytic activity. The new method can readily be used to construct larger complexes that contain more than six enzymes.

It is noted that the bacterial cells described herein do not require in vitro assembly of the complex. Thus, for example, no purified cellulases must be added to the cells to form the minicellulosome. It is also noted that it is believed the B. subtilis cells described herein (e.g., in Example 2) exhibit the highest cellulolytic activity of any recombinant microorganism yet reported.

The scaffold extension method described herein provides a simple approach to quickly increase the number of enzymes that are housed in multi-cellulase complexes displayed on the surface of a Gram-positive bacterium (e.g., B. subtilis). By using an extended scaffoldin approach, larger cellulase complexes can be assembled using smaller proteins. This is advantageous because it overcomes problems associated with secreting and folding of larger polypeptides. With this new system, Gram-positive bacteria (e.g., B. subtilis) can be engineered to display complexes that contain 4 or more, 5 or more, 6 or more, 7 or more, 8, or more, 9 or more, 10 or more, 11 or more, or 12 or more enzymes.

The new B. subtilis cells displaying the extended, six enzyme complex described herein in Example 2 exhibit improved cellulolytic activity and are therefore of greater potential use. Importantly, the method described herein is general, and can therefore be applied to construct cells that contain complexes that house more than six enzymes.

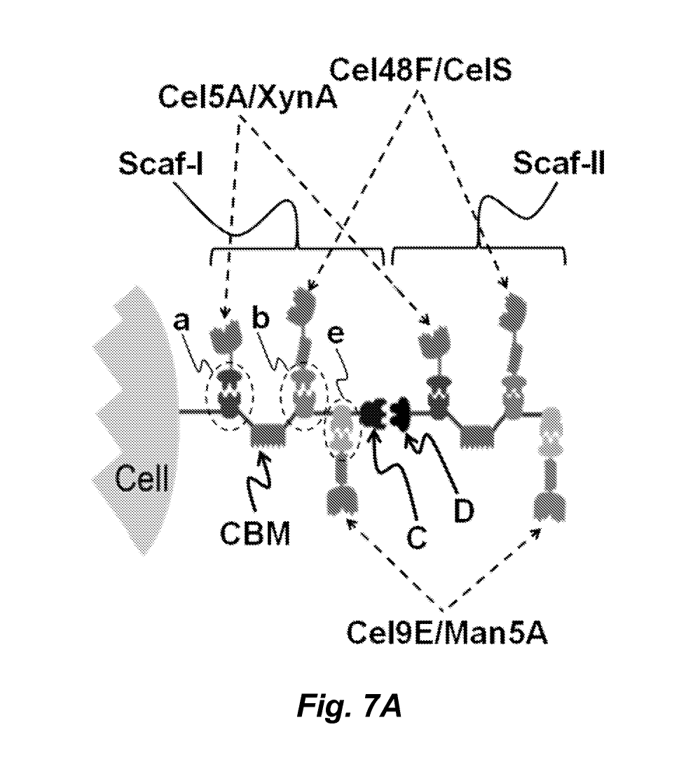

One illustrative, but non-limiting embodiments is shown in FIG. 7A and described in detail herein in Example 2.

Briefly, strain TDA21 was generated to co-express nine proteins: (1) the SrtA sortase from B. anthracis, (2) a chimeric scaffoldin (Scaf-I) composed of three type-I cohesin modules and one type-II cohesin module that is covalently attached to the cell wall by SrtA, (3) a second chimeric scaffoldin (Scaf-II) composed of three type-I cohesin modules and a type-II dockerin module, and (4-9) six dockerin-cellulase fusion proteins that bind to the scaffoldin non-covalently via species-specific dockerin-cohesin interactions (see, e.g., Table 7 in Example 2). The six cellulases were derived from C. cellulolyticum and C. thermocellum and have complementary cellulose degrading activities: Cel5A (endoglucanase/xylanase, family 5 glycoside hydrolase (GH)), Cel48F (processive endoglucanase, family 48 GH), Cel9E (exoglucanase, family 9 GH), CelS (exognlucanase, family 48 GH), Man5A (mannanase, family 5 GH), and XynA (xylanase, family 11 GH). Each protein component of the minicellulosome also contained an N-terminal signal sequence enabling them to be exported to the cell surface. The Scaf-I and Scaf-II proteins contain cohesin modules derived from C. cellulolyticum, C. thermocellum, and Ruminococcus flavefaciens that selectively bind to their cognate dockerin modules fused to Cel5A, Cel48F, Cel9E, CelS, Man5A, and XynA (FIG. 7A and Table 7). Scaf-I, Scaf-II, and the Cel9E enzyme also contain family 3 and 4 carbohydrate binding modules (CBM), respectively, which tether the enzyme complex to the cellulose component of the biomass. The scaf-I, scaf-II, and srtA genes were integrated into the thrC locus of the chromosome, while genes expressing the six cellulase-dockerin fusion proteins are expressed from the pHCMC05-based plasmid pCellulase and pDG148-based plasmid pSXM. All genes are expressed from a Pspac promoter and are IPTG inducible.

The construct described in Example 2 is intended to be illustrative and non-limiting. Using the methods described herein numerous other minicellulosomes comprising 4 or more, 5 or more, 6 or more, 7 or more, 8, or more, 9 or more, 10 or more, 11 or more, or 12 or more enzymes can be constructed and expressed on Gram-positive bacteria.

It has recently been noted that only twelve biomass-derived building blocks are needed to produce a range of commercial products. It is demonstrated that the protein display systems described herein can be used to create B. subtilis strains that can grow on plant biomass. Therefore, metabolic engineering of the cells described herein can create a consolidated bioprocessing microbe (CBP) that produces these biocommodities or building-blocks from cheap plant biomass. An immediate application is to introduce the cellulolytic system into existing microorganisms (e.g., bacteria) that are already used industrially to produce commercial products. This would enable the products to be produced from biomass and would significantly reduce costs.

Another illustrative, but non-limiting application of the systems described herein would be to use to use the biomass degrading cells as a replacement for enzyme cocktails that are currently used in industry to degrade biomass. The cells could be produced more cheaply than the enzymes and thereby reduce the costs associated with degrading biomass into its component sugars. An immediate application is lignocellulose degradation needed to produce lignocellulosic ethanol. Cells displaying the minicellulosomes would degrade the biomass into component sugars, which would then be used in downstream ethanol producing fermentation reactions performed by S cerevisiase.

The system we have developed can also be used to engineer other species of Gram-positive bacteria to convert them into CPB or microbes that are dedicated to degrading biomass into sugars.

### Gram-Positive Microorganisms.

In various embodiments it is contemplated that the display methods described herein can be used with virtually any microorganism capable of exploiting a sortase A transpeptidase reaction to anchor a protein to the cell surface. In various embodiments the microorganism is a Gram-positive microorganism (e.g., a Gram-positive bacterium).

The term “Gram-positive bacteria” generally refers to bacteria that are stained dark blue or violet by Gram staining Gram-positive microorganisms are well known to those of skill in the art. Gram-positive bacteria are generally divided into the Actinobacteria and the Firmicutes. The Actinobacteria or actinomycetes are a group of Gram-positive bacteria with high G+C ratio. They include some of the most common soil bacteria. Other Actinobacteria inhabit plants and animals and including some pathogens, such as Mycobacterium, Corynebacterium, Nocardia, Rhodococcus and a few species of Streptomyces. The majority of Firmicutes have Gram-positive cell wall structure. Illustrative Gram-positive bacteria include, but are not limited to Acetobacterium, Actinomyces (e.g., A. israelii), Arthrobacter, Bacillus (e.g., B. subtilis), Bifidobacterium, Clostridium, Clostridium spp. (e.g., C. perfringens, C. septicum, C. tetanomorphum), Corynebacterium, Enterococcus, Eubacterium, Frankia, Heliobacterium, Heliospirillum, Lactobacillus, Lactococcus, Leuconostoc, Listeria, Listeria spp., Megasphaera, Micrococcus spp., Micromonospora, Mycobacterium, Nocardia, Pectinatus, Pediococcus, Propionibacterium, Selenomonas, Sporomusa, Staphylococcus spp. (e.g., S. aureus), Streptococcus spp., (e.g., S. pneumoniae, B group streptococci), Streptomyces, and Zymophilus. Similarly, sortases, secretion signals cell wall sorting signals can include, but are not limited to, those derived from any Gram-positive microorganism.

In certain embodiments the bacterial host is selected from the group of non-pathogenic and/or non-invasive, Gram-positive bacteria consisting of Lactobacillus, Lactococcus, Pediococcus, Carnobacterium, Bifidobacterium, Oenococcus, Bacillus subtilis, Streptococcus thermophilus, and other non-pathogenic and/or non-invasive Gram-positive bacteria known in the art. In certain embodiments the bacterial host cell preferably is a Gram-positive bacterium, more preferably a Gram-positive bacterium that belongs to a genus selected from the group consisting of Lactobacillus, Lactococcus, Leuconostoc, Carnobacterium, Bifidobacterium, Bacillus, Streptococcus, Propionibacterium, Oenococcus, Pediococcus, Enterococcus. In certain embodiments the bacterial host cell is a bacterium that belongs to a species selected from the group consisting of L. acidophilus, L. amylovorus, L. bavaricus, L. brevis, L, caseii, L. crispatus, L. curvatus, L. delbrueckii, L. delbrueckii subsp. bulgaricus, L. fermentum, L. gallinarum, L. gasseri, L. helveticus, L. jensenii, L. johnsonii, L. minutis, L. murinus L. paracasei, L. plantarum, L. pontis, L. reuteri, L. sacei, L. salivarius, L. sanfrancisco, Lactobacillus ssp., C. piscicola, B. subtilis, Leuconostoc mesenteroides, Leuconoctoc lactis, Leuconostoc ssp, L. lactis subsp. lactis, L. lactis subsp. cremoris, Streptococcus thermophilus, B. bifidum, B. longum, B. infantis, B. breve, B. adolescente, B. animalis, B. gallinarum, B. magnum, and B. thermophilus.

### Methods of Engineering Microorganisms.

As indicated above, in certain embodiments, microorganisms are engineered to contain a nucleic acid construct that exploits a sortase pathway to covalently anchor a protein to the surface of the cell. In certain embodiments the nucleic acid construct encodes a protein comprising one or more, preferably two or more cohesin domains attached to a secretory signal sequence (e.g., at the N-terminus of the protein) and a cell wall sorting signal (e.g., at the carboxyl terminus of the protein). In certain embodiments the same or additional constructs encode dockerin domains attached to a cellulolytic enzyme. The dockerin domains are selected to mate/bind with the cohesin domains on the “scaffoldin” protein. As described herein and illustrated in the examples, the entire system is designed to create a self-assembling minicellulosome.

In one illustrative, but non-limiting embodiment, a microorganism is transfected with the construct(s) and as encoded protein is transcribed it is displayed on the surface of the microorganism, e.g., through the transpeptidase reaction mediated by a sortase. The sortase can be an endogenous sortase expressed by the microorganism. In certain embodiments the sortase can be a sortase that is encoded by the same or another nucleic acid construct transfected into the microorganism. In certain embodiments the sortase is a sortase found in the subject microorganism, and in certain embodiments, the sortase is a sortase characteristic of a different microorganism.

In certain embodiments, particularly where a minicellulosome is to be expressed, the same construct or a different nucleic acid construct is provided that encodes one or more dockerins each attached to a different enzyme (e.g., cellulolytic enzyme) as described above.

Methods of making the nucleic acid constructs described herein are well known to those of skill in the art, and specific methods are illustrated in the examples. Cloning and bacterial transformation methods, DNA vectors and the use of regulatory sequences are well known to the skilled artisan and may for instance be found in Current Protocols in Molecular Biology, F. M. Ausubel et al, Wiley Interscience, 2004, incorporated herein by reference.

Many embodiments, of the methods and constructs described herein utilize an expression vector containing a nucleotide sequence that encodes the protein(s) of interest, a cell wall sorting signal and a secretion signal. Suitable expression vectors include, but are not limited to baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, and any other vectors specific for specific hosts of interest. Such vectors include chromosomal, nonchromosomal and synthetic DNA sequences, and may comprise a full or mini transposon for the integration of a desired DNA sequence into the host chromosome. Examples of tranposons include but are not limited to TN5, TN7, and TN10, as well as the engineered transposomes from Epicentre (www.epicentre.com).

Numerous suitable expression vectors are known to those of skill in the art, and many are commercially available. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXTI, pSGS (Stratagene), pSVK3, pBPV, pMSG, and pSVLSV40 (Pharmacia). However, any other plasmid or other vector, with or without various improvements for expression, may be used so long as it is compatible with the host cell.

In certain embodiments the subject vectors will contain a selectable marker gene. In certain embodiments this gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli, and the like.

The vector(s) of interest can be transfected into and propagated in the appropriate host. Methods for transfecting the host cells with the genomic DNA vector can be readily adapted from those procedures which are known in the art. For example, the vector can be introduced into the host cell by such techniques as the use of electroporation, precipitation with DEAE-Dextran or calcium phosphate, or lipofection.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 2004/0131637), apagC promoter (Pulkkinen and Miller (1991) J. Bacteriol., 173 (1): 86-93; Alpuche-Aranda et al. (1992) Proc. Natl. Acad. Sci. U.S.A. 89(21): 10079-83), a nirB promoter (Harborne et al. (1992) Mol. Micro. 6: 2805-2813), and the like (see, e.g., Dunstan et al. (1999) Infect. Immun. 67: 5133-5141; McKelvie et al. (2004) Vaccine 22: 3243-3255; Chatfeld et al. (1992) Biotechnol. 10: 888-892, and the like); a sigma70 promoter, e.g., a consensus sigma70 promoter (see, e.g., GenBank Accession Nos. AX798980, AX798961, and AX798183); a stationary phase promoter, e.g., a dps promoter, a spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035-7056); and the like.

In certain embodiments the nucleic acid constructs of interest are operably linked to an inducible promoter or to a constitutive promoter. Inducible and constitutive promoters are well known to those of skill in the art.

Where the host cell is genetically modified to express two or more gene products (e.g., a sortase and a protein comprising a sorting signal), nucleotide sequences encoding the two or more gene products will in some embodiments each be contained on separate expression vectors and in some embodiments contained in the same vector.

Where nucleotide sequences encoding the two or more gene products are contained in a single expression vector, in some embodiments, the nucleotide sequences will be operably linked to a common control element (e.g., a promoter), e.g., the common control element controls expression of all gene product-encoding nucleotide sequences on the single expression vector. In some embodiments, the nucleotide sequences encoding different gene products are operably linked to different control element(s) (e.g., promoter(s)). In some embodiments, one of the nucleotide sequences will be operably linked to an inducible promoter, and one or more of the other nucleotide sequences will be operably linked to a constitutive promoter.

As described above, the nucleic acid constructs may be introduced into the host cell as extra-chromosomal genetic materials that can replicate themselves (e.g., plasmids), or as genetic material integrated into the host genome. Regardless of whether the heterologous genes are integrated into the host genome, or exist as extra-chromosomal genetic materials, the optimal expression of the constructs heterologous genes belonging to a new metabolic pathway can on occasion benefit from coordinated expression of such genes, tight control over gene expression, and consistent expression in all kinds of host cells.

Methods and systems are provided that fine-tune the expression of heterologous genes, which in turn allow reproducible manipulation of metabolism in model microbes, such as E. coli, Bacillus subtillis, and Aspergillus nidulans. These methods allow balanced expression of the heterologous genes (e.g., those encoding the cellulosome) by techniques such as fine-tuning mRNA stability, the use of inducible promoters of various strengths, etc. See, for example, Keasling et al., New tools for metabolic engineering of E. coli. In Metabolic Engineering, S.-Y. Lee and E. T. Papoutsakis, eds. Marcel Dekker, New York, N.Y. (1999); Keasling, Gene-expression tools for the metabolic engineering of bacteria. Trends in Biotechnology 17:452-460, 1999; Martin et al., Redesigning cells for production of complex organic molecules. ASM News 68: 336-343, 2002 (all incorporated herein by reference).

While the foregoing discussion and examples below focus on Gram-positive bacteria and Sortase A, it will be appreciated that the methods described herein are amenable for use in any microorganism in which a sortase is found or can be expressed and is functional. Thus, for example, sortase enzymes have also been identified in the gram-negative organisms Bradyrhizobium japonicum, Colwellia psychroerythraea, Microbulbifer degradans, Shewanella oneidensis, and Shewanella putrefasciens, as well as in Methanobacterium thermoautotrophicum, a thermophilic archaeon (Pallen et al. (2003) Curr. Opin. Microbiol. 6: 519-527.). The use of the methods described herein with any of these organisms is also contemplated.

The foregoing methods and constructs are intended to be illustrative and not limiting. Using the teachings provided herein, numerous proteins, enzymes, minicellulosomes and the like can be stably displayed on the surface of a microorganism.

### EXAMPLES

The following examples are offered to illustrate, but not to limit the claimed invention.

### Example 1

### Recombinant Bacillus subtilis that Crows on Untreated Plant Biomass

This example describes the engineering of B. subtilis to display a cell wall attached minicellulosome that assembles spontaneously. We show that these recombinant cells degrade both pretreated and untreated forms of lignocellulosic biomass, enabling them to grow robustly when these substances are provided as a primary nutrient source. This is an important step in the development of a CBP that can cost-efficiently convert biomass into valuable commodities.

### Materials and Methods

Construction of B. Subtilis Strains.

A description of the strains and plasmids created in this study can be found in Table 6. The genes encoding srtA and scaf were integrated into the thrC locus by homologous recombination using the pSrtA/Scaf plasmid derived from vector pBL112 (Lanigan-Gerdes et al. (2007) Molecular Microbiol., 65: 1321-1333). Both genes are IPTG (Isopropyl-β-D-1-thiogalactopyranoside) inducible under the P_{spac} promoter. srtA encodes the B. anthracis sortase A and has been described previously (Anderson et al. (2011) Appl. Environ. Microbiol., 77: 4849-4858). The scaf gene encodes a fusion protein that contains three type I cohesin modules derived from three different bacterial species: C. cellulolyticum (CipC), C. thermocellum (CipA), and R. flavefaciens (ScaB) (Fierobe et al. (2005) J. Biol. Chem., 280: 16325-16334). It also contains a family 3 carbohydrate binding module (CBM) from C. thermocellum CipA and the cell wall sorting signal (CWSS) from Staphylococcus aureus fibronectin binding protein B (Anderson et al. (2011) Appl. Environ. Microbiol., 77: 4849-4858). The genes encoding the cellulase enzymes used in this study have been described previously, and were cloned into pHCMC05 (Bacillus Genetic Stock Center) to create plasmid pCellulase (Fierobe et al. (2005) J. Biol. Chem., 280: 16325-16334). Plasmid pCellulase contains genes encoding the three cellulase enzymes. cel9E encodes a fusion protein that contains a N-terminal VSV-g epitope tag, a CBM, immunoglobulin-like domain, a family 9 glycoside hydrolase (GH) domain, and the R. flavefaciens type I dockerin module. cel48F encodes an N-terminal Myc epitope tag, a family 48 GH, and a type I dockerin module from C. thermocellum. cel5A contains a family 5 GH with its native type I dockerin module and a C-terminal hexahistidine (His₆) tag. In addition, a nucleotide sequence encoding a ribosome binding site and secretion signal derived from B. subtilis phrC was appended to scaf cel9E, cel48F, and cel5A. Similar methods were used to create plasmids pCel5A, pCel9E, pCel48F, pCel5A/9E, pCel5A/48F, and pCel9E/48F that contain one or two cellulase genes. Strains containing pSrtA/Scaf and/or plasmids encoding the cellulase genes were generated by transforming the plasmids into B. subtilis BAL2238 using standard methods, and involve plating on Luria-Bertani (LB) agar plates containing 1 μg/ml erythromycin or 5 μg/ml chloramphenicol (Anderson et al. (2011) Appl. Environ. Microbiol., 77: 4849-4858).

TABLE 6
Bacillus subtilis strains and plasmids used in this Example.

| | | | Reference |
|---|---|---|---|
| Strain | Relevant genotype | Phenotype^{a} | or Source |
| BAL2238 | ΔwprA::hyg trpC2 pheA1 | | 33 |
| TDA10 | pSrtA/Scaf | SrtA, Scaf^{b,c} | This study |
| TDA11 | pSrtA/Scaf; pCel5A | SrtA, Scaf, Cel5A^{b,c,d} | This study |
| TDA12 | pSrtA/Scaf; pCel9E | SrtA, Scaf, Cel9E^{b,c,e} | This study |
| TDA13 | pSrtA/Scaf; pCel48F | SrtA, Scaf, Cel48F^{b,c,f} | This study |
| TDA14 | pSrtA/Scaf; pCel9E/48F | SrtA, Scaf, Cel9E, Cel48F^{b,c,d,f} | This study |
| TDA15 | pSrtA/Scaf; pCel5A/9E | SrtA, Scaf, Cel5A, Cel9E^{b,c,d,e} | This study |
| TDA16 | pSrtA/Scaf; pCel5A/48F | SrtA, Scaf, Cel5A, Cel48F^{b,c,e,f} | This study |
| TDA17 | PSrtA/Scaf; pCellulase | SrtA, Scaf, Cel5A, Cel9E, | This study |
| | | Cel48F^{b,c,d,e,f} | |
| TDA18 | pCellulase | Cel5A, Cel9E, Cel48F^{d,e,f} | This study |
| TDA19 | pScaf | Scaf^{c} | This study |

| | | Reference |
|---|---|---|
| Plasmid | Relevant Characteristics^{g} | or Source |
| pBL112 | B. subtilis-E. coli shuttle plasmid that integrates into thrC | 38 |
| | locus in the B. subtilis genome, IPTG inducible promoter, | |
| | Amp^{r}, Ery^{r g} | |
| pHCMC05 | B. subtilis expression plasmid with IPTG inducible promoter, | BCSC^{h} |
| | Amp^{r}, Cm^{r} | |
| pSrtA/Scaf | B. anthracis srtA and scaf in pBL112 | This study |
| pScaf | scaf in pBL112 | This study |
| pCel5A | C. cellulolyticum cel5A in pHCMC05 | This study |
| pCel9E | C. cellulolyticum cel9E in pHCMC05 | This study |
| pCel48F | C. cellulolyticum cel48F in pHCMC05 | This study |
| pCel9E/48F | C. cellulolyticum cel5A, C. cellulolyticum cel48F in | This study |
| | pHCMC05 | |
| pCel5A/9E | C. cellulolyticum cel5A, C. cellulolyticum cel9E in | This study |
| | pHCMC05, | |
| pCel5A/48F | C. cellulolyticum cel5A, C. cellulolyticum cel48F in | This study |
| | pHCMC05 | |
| pCellulase | C. cellulolyticum cel5A, C. cellulolyticum cel9E, | This study |
| | C. cellulolyticum cel5A in pHCMC05 | |

^{a}Protein(s) expressed by the strains.
^{b}Flag-tagged full length sortaseA from B. anthracis strain Ames
^{c}Scaf contains a three cohesin containing polypeptide (type I cohesins from C. thermocellum CipA, C. cellulolyticum CipC, and R flavefaciens ScaB) and a family 3 CMB that is anchored to the cell by SrtA vi the S. aureus fibronectin binding protein cell wall sorting signal.
^{d}Cel % A contains C. cellulolyticum Cel5A endoglucanase/xylanase fused to its native dockerin, an N-terminal secretory peptide, and a C-terminal His₆-tag.
^{e}Cel9E contains the C. cellulolyticum Cel9E exoglucanase fused to an R. flavefaciens type-I dockerin, an N-terminal secretory peptide, and VSV-G epitope tag.
^{f}Cel48F contains C. cellulolyticum Cel48F processive endoglucanase fused to a C. thermocellum type I dockerin, and N-terminal secretory peptide, and Myc epitope tag.
^{g}Amp^{r}, ampicillin resistance; Ery^{r}, erythromycin resistance; Cm^{r}, chloramphenicol resistance.
^{h}Bacillus Genetic Stock Center.

Cell Fractionation and Immunoblot Analysis.

Cultures were grown overnight at 37° C. to saturation in 5 ml LB supplemented with 5 μg/ml chloramphenicol. A total of 500 μl of the overnight culture was then used to inoculate 50 ml of similar media and grown at 37° C. until the cells reached an optical density at 600 nm (OD₆₀₀) of 0.1. At this point, IPTG was added to 1 mM to induce expression of the srtA, scaf, and cellulase genes. After the cells reached saturation, they were collected by centrifuging at 3,000×g for 10 min, washed with 1 ml STM buffer (50 mM Tris-HCl, pH 8.0, 25% sucrose, 5 mM MgCl₂), centrifuged at 3,000×g for 5 min, and then re-suspended in STM such that the cell densities between samples were identical (OD₆₀₀ ˜10). The cells were then fractionated by incubating with lysozyme (500 μg/ml) for 30 min at 37° C. to solubilize the cell walls. The suspensions were then centrifuged at 20,000×g to pellet the protoplasts, and the supernatant, which contains solubilized cell wall components, was collected. Secreted proteins were also collected from the spent growth medium, which was filtered through a 0.2 μm filter to remove cells. The proteins in the medium were precipitated with 10% trichloroacetic acid, centrifuged, and the pellet re-dissolved in water for immunoblot analysis. Samples of the solubilized cell walls (equivalent to 2.5×10⁴ cells) and precipitated secreted protein (equivalent to protein secreted by 2.5×10⁴ cells) were separated by sodium dodecyl sulfate-polyacrylamide gel electrophoresis (SDS-PAGE) and transferred to polyvinylidene fluoride (PVDF) membranes. Standard procedures for immunoblot analysis were used as described previously (Anderson et al. (2011) Appl. Environ. Microbiol., 77: 4849-4858). Anti-His₆ (0.25 μg/ml dilution for 1 h, Abgent) and anti-Myc (1 μg/ml dilution for 1 h, Syd Labs) primary antibodies were used to probe for Cel5A and Cel48F, respectively, and visualized using horseradish peroxidase conjugated to a rabbit anti-mouse immunoglobulin G secondary antibody (1:50,000 dilution for 1 h, Sigma). Cel9E was visualized using an anti-VSV-g primary antibody (0.5 μg/ml dilution for 1 h, Acris Antibodies) and horseradish peroxidase conjugated to a goat anti-biotin secondary antibody (1:20,000 dilution for 1 h, Cell Signaling).

Biomass Growth Studies.

Untreated corn stover, switchgrass, and hatched wheat straw were ground, washed with deionized water, and dried in an oven at 100° C. For some assays, the corn stover was first pretreated using dilute sulfuric acid as described previously (Jensen et al. (2010) Bioresource Technol., 101: 2317-2325). Briefly, 90 ml of 0.8% sulfuric acid was incubated with 3 g ground corn stover. A laboratory autoclave was then utilized to heat the corn stover-sulfuric acid suspension at 120° C. for 15 min. Following heating, the suspension was neutralized by washing with deionized water and dried in a laboratory oven at 100° C. Strains were tested for their ability to grow on untreated and pretreated biomass. Colonies from agar plates were used to inoculate a 5 mL LB culture supplemented with 1 μg/ml erythromycin and/or 5 μg/ml chloramphenicol, in order to select for srtA/scaf integrants and cells containing plasmids encoding cellulase enzymes, respectively. After 8 hours of growth at 37° C., 100 μl of each culture was transferred into 5 ml of M9 medium that contained 0.5% w/v glucose (Zhang et al. (2011) Metabolic Engineering 13: 364-372). The media also contained 0.004% tryptophan, 0.004% phenylalanine, and 0.004% threonine as the parent strain is auxotrophic for these amino acids. After 16 hours of growth, 100 μl of each culture was used to inoculate a 5 ml culture that contained biomass as the sole carbon source. This media consists of M9 minimal media and 0.5% w/v treated/untreated biomass. In control experiments the biomass was replaced with 0.5% w/v glucose. To induce protein expression 1 mM IPTG was added immediately after inoculating the biomass-containing culture. The OD₆₀₀ of the cultures were measured over a 72 hour period. In addition to monitoring the cell density (OD₆₀₀), colony forming units (CFUs) of strains TDA17 and TDA18 cultured in the presence of glucose or biomass were determined by plating 100 μl of the 10²-10⁶ dilutions onto LB plates supplemented with 5 μg/ml chloramphenicol and the resultant colonies that grew counted. Cells assayed for growth include those capable of displaying three (strain TDA17), two (strains TDA14 (Cel9E+Cel48F), TDA15 (Cel5A+Cel9E) and TDA16 (Cel5A+Cel48F)), or one cellulase enzyme (strains TDA11 (Cel5A), TDA12 (Cel9E) and TDA13 (Cel48F)), or cells only capable of secreting the enzymes (strain TDA18). Growth assays were performed in triplicate and the standard deviation was used as an estimate of the error.

### Whole-Cell and Cellulase Cocktail Sugar Release Assays.

Cells induced for protein expression were grown to saturation in LB media as described above. They were then centrifuged at 3,000×g for 10 min, re-suspended in Assay buffer (20 mM Tris-acetate, pH 6.0, 1 mM CaCl₂, 0.1% sodium azide), re-centrifuged, and the final cell pellet was re-suspended in Assay buffer. Lignocellulosic biomass was then added to the cell suspension such that there was a total of ˜15 mg of cell displayed cellulase enzymes per gram of biomass; 10 ml suspensions containing cells at an OD₆₀₀ of 2.5 were incubated with 60 mg of biomass at 37° C. with shaking (Banerjee et al. (2010) Biotechnol. Bioengineer., 106: 707-720; Banerjee et al. (2010) Bioresource Technol., 101: 9097-9105). In some instances, exogenous β-glucosidase (Sigma) was added to the cell-biomass mix (1 mg/g biomass), and the amount of cell suspension used was correspondingly adjusted to maintain a ratio of ˜15 mg enzyme per g biomass. For the assays performed using commercially available cocktails, a mixture containing 13.5 mg of Ctec2 and 1.5 mg of Htec2 enzyme cocktails (Novozymes Inc.) per gram of biomass was shaken in 10 ml of assay buffer at 37° C. To measure the amount of total biomass degraded, the cell-biomass mixture was removed at various times from the shaker and the insoluble biomass was allowed to settle. After decanting the cell, the residual biomass was washed with deionized water, decanted again, and the insoluble fraction dried (100° C. for 1 h). Measurement of reducing sugars released into the medium was accomplished as described previously and made use of dinitrosalicylic acid (glucose was used as the standard) (Anderson et al. (2011) Appl. Environ. Microbiol., 77: 4849-4858; Tsai et al. (2009) Appl. Environ. Microbiol., 75: 6087-6093). Glucose was assessed using a glucose assay kit (Eton Biosciences) that makes use of the glucose oxidase enzyme and followed procedures outlined by the manufacturer (Banerjee et al. (2010) Biotechnol. Bioengineer., 106: 707-720; Banerjee et al. (2010) Bioresource Technol., 101: 9097-9105). Xylose release was analyzed using phloroglucinol (Fisher) as described previously (Eberts et al. (1979) Clinical Chem., 25: 1440-1443; Akin and Rigsby (2008) Appl. Biochem. Biotechnol., 144: 59-68). Control experiments made use of strain TDA18 which lacks scaf and srtA, but contains the cellulase expressing plasmid resulting in cellulase secretion. Assays were performed in triplicate and the standard deviation was used as an estimate of the error.

Determination of Surface-Displayed Scaf Levels and the Degree of Saturation.

For these studies full-length Cel5A, Cel9E, and Cel48F were overexpressed in E. coli and purified. Plasmids expressing each protein were created by sub-cloning their genes into plasmid pET28a using standard methods. After transformation into E. coli strain BL21(DE3), the proteins were over-expressed using standard procedures described by Novagen. For protein purification, cell pellets were re-suspended in lysis buffer (25 mM Tris-Cl, pH 7.0, 250 mM NaCl, 25 mM CaCl₂), lysed by sonication, and the supernatant collected by centrifugation at 20,000×g for 30 min. The supernatant was then passed through Co-NTA resin, washed with 10 column volumes of lysis buffer supplemented with 10 mM imidazole, and eluted in lysis buffer supplemented with 100 mM imidazole. The proteins were then buffer exchanged into binding buffer (20 mM Tris-Cl pH 6.0, 2 mM CaCl₂), concentrated, and the amount of protein quantified using a BCA assay.

To estimate the amount of Scaf displayed per cell, purified Cel5A was added to a known number of cells that display Scaf attached to the cell wall (strain TDA10). The amount of Cel5A adhered to Scaf was then estimated by measuring the cellulolytic activity of the cells. Briefly, a 50 ml LB culture of strain TDA10 supplemented with 1 μg/ml erythromycin was grown to an OD600 of 0.1, and IPTG was added to a final concentration of 1 mM to induce SrtA and Scaf expression. After 4 hrs, the cells were collected by centrifugation (3,000×g for 5 minutes), and re-suspended in binding buffer. To ensure that non-covalently bound Scaf was removed from the cells, this wash step was repeated. Increasing amounts of purified Cel5A was added to 2.4×10¹⁰ cells and incubated on ice for 1 hr. They were then centrifuged at 3,000×g for 5 minutes, the supernatant was removed, and the cells were re-suspended in 1 ml of binding buffer. This washing step was performed twice. The cells were then pelleted by centrifugation, the supernatant was removed, and the cells were re-suspended in 1 ml 0.5% CMC (dissolved in assay buffer). The amount of reducing sugar produced was then determined using DNS as described above. Control experiments were performed using strain TDA19, which does not produce SrtA needed to attach Scaf to the cell wall.

To determine if the cohesin domains within Scaf are saturated with enzymes TDA17 cells displaying minicellulosomes were exposed to purified enzymes and an immunoblot was performed to determine if they could bind additional protein. A 50 ml culture of cells was induced to express the minicellulosome as described above. The cells were collected by centrifugation and the pellet re-suspended in binding buffer. This procedure was repeated to wash the cells. A total of 5×10¹⁰ cells were then incubated with an excess of Cel5A, Cel9E, and Cel48F; to the cells, 2 mg of each purified enzyme was added. After incubation on ice for 1 hr the cells were then fractionated and subjected to immunoblot analysis as described above. Additional control experiments were performed using strains TDA10, TDA18 and TDA19 instead of TDA17.

### Results

B. subtilis Cells Display a Self-Assembled Minicellulosome.

Cells that display ex vivo assembled minicellulosomes are of questionable practicality for industrial applications; thus, we engineered B. subtilis to display a minicellulosome that self-assembles (FIG. 1A). Strain TDA17 was generated to co-express five proteins: the SrtA sortase from B. anthracis, a chimeric scaffoldin (Scaf) composed of three cohesin modules that is covalently attached to the cell wall by SrtA, and three dockerin-cellulase fusion proteins that bind to the scaffoldin non-covalently via species-specific dockerin-cohesin interactions (Table 6). The three cellulases were derived from C. cellulolyticum and have complementary cellulose degrading activities: Cel5A (endoglucanase/xylanase, family 5 glycoside hydrolase (GH)), Cel48F (processive endoglucanase, family 48 GH), and Cel9E (exoglucanase/endoglucanase, family 9 GH). Each protein component of the minicellulosome also contains an N-terminal signal sequence enabling them to be exported to the cell surface. The Scaf protein contains cohesin modules derived from C. cellulolyticum, C. thermocellum, and Ruminococcus flavefaciens, which selectively bind to their cognate dockerin modules fused to Cel5A, Cel48F, and Cel9E, respectively (FIG. 1A and Table 6). Scaf and the Cel9E enzyme also contain family 3 and 4 carbohydrate binding modules (CBM), respectively, which tether the enzyme complex to the cellulose component of the biomass. The scaf and srtA genes are integrated into the thrC locus of the chromosome, while genes expressing the three cellulase-dockerin fusion proteins are expressed from the pHCMC05-based plasmid pCellulase. All genes are expressed from a P_{spac} promoter and are IPTG inducible.

Cell fractionation and immunoblotting experiments were performed to confirm that the enzyme components self-assemble to form a minicellulosome on the cell surface (FIG. 1B). Strain TDA17 was induced with IPTG to express genes encoding SrtA and the four minicellulosome components (Scaf, Cel5A, Cel48F, and Cel9E). After growth to saturation, the cell wall (CW) and secreted protein (Sec) fractions were isolated and the presence of the Cel9E, Cel48F, and Cel5A enzymes probed using appropriate antibodies. A similar set of experiments was performed with strain TDA18, which contains the pCellulase plasmid, but lacks the scaf and srtA genes. When all genes were expressed, the three enzyme fusions associated with the cell wall fraction (FIG. 1B, TDA17). In the absence of SrtA and Scaf, the cellulases were secreted into the medium and not anchored to the cell wall (FIG. 1B, TDA18). Specific enzyme display was further confirmed using strains that expressed the scaf and srtA genes, and either Cel5A (TDA11) or Cel9E and Cel48F (TDA14) (FIG. 1B). In these strains, the appropriate enzymes were associated with the cell wall in a sortase-dependent manner. Interestingly, in addition to being associated with the cell wall fraction, the cellulases were also secreted into the medium; they may be secreted because they were overexpressed such that they saturated the available Scaf binding sites on the cell surface and/or because a fraction of the secreted cellulase enzymes failed to refold properly. Interestingly, the band for Cel48F is less intense than the bands for Cel5A and Cel9E. However, Cel48F would appear to be as abundant as these other enzymes on the cell surface as Scaf is saturated with each enzyme (described immediately below). Therefore, the lower intensity of the Cel48F band may be caused by the unique primary antibody that is used to detect it.

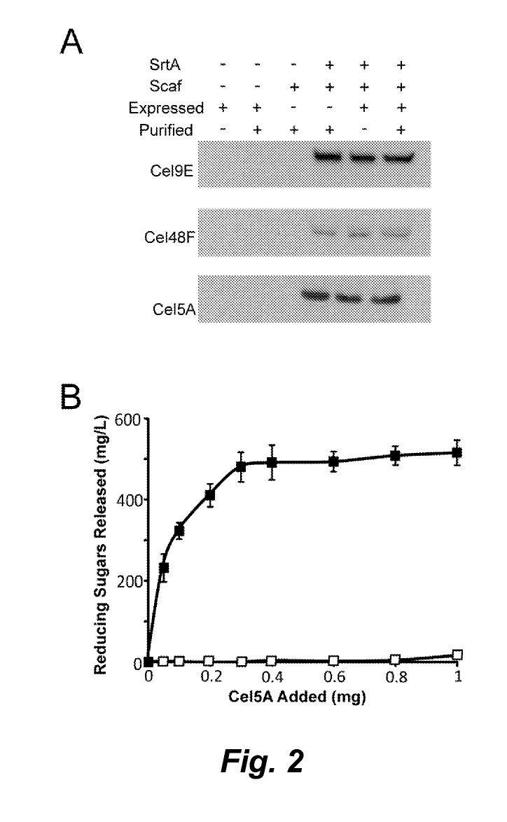

Experiments were performed to quantify the number of displayed complexes and to determine if the Scaf proteins were saturated with enzymes. Data in FIG. 2, panel A indicates that Scaf is saturated with the enzymes. Cells displaying a completely assembled minicellulosome (TDA17) were incubated with purified enzymes produced in E. coli and an immunoblot of the cell wall fraction was performed to determine if binding occurred. The addition of purified enzymes to cells that already express the cellulase proteins does not change the amount of cell-associated enzyme. This indicates that no additional protein binds to the cell, presumably because Scaf is already saturated with enzymes that are expressed from the cell. Importantly, control experiments indicate that the purified enzymes are able to interact with cells capable of only displaying covalently attached Scaf (TDA10) and that non-Scaf mediated binding to the cells does not occur. To estimate how many Scaf proteins are displayed on each microbe a total of 2.4×10¹⁰ TDA10 cells were incubated with differing amounts of purified Cel5A enzyme of known specific activity (FIG. 2, panel B). The amount of Scaf bound by Cel5A was then determined indirectly by measuring the amount of cell-associated Cel5A enzyme activity. The results of this analysis indicate that each cell displays ˜150,000 Scaf proteins that are competent to bind Cel5A.

Degradation and Growth on Dilute Acid Pretreated Corn Stover.

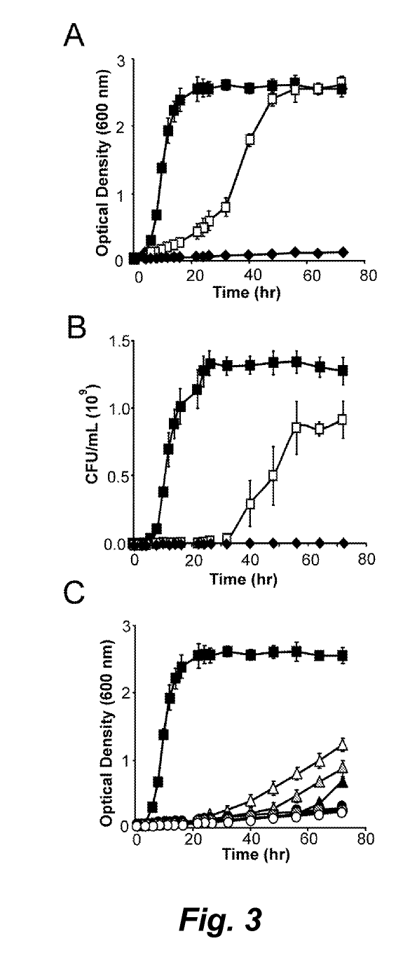

Recombinant B. subtilis displaying the full-complement of enzymes in its minicellulosome (strain TDA17) grew when cultured aerobically in minimal media containing 0.5% w/v dilute acid pretreated corn stover as the sole carbon source (FIG. 3, panel A). Based on OD₆₀₀ measurements of the cultures the cells achieved densities that are similar to cells cultured in 0.5% w/v glucose, but grew more slowly and with a longer lag phase. However, it has been noted previously that the biomass or its degradation products can contribute to the OD₆₀₀ of the solution (Lynd and Zhang (2002) Biotechnol. Bioengineering 77: 467-475). We therefore measured the colony forming units per milliliter (CFU/mL) of cell cultures that were grown on either biomass or glucose (FIG. 3, panel B). This data indicates that after 72 hours the cellulosome producing cells grown on biomass reach 1×10⁹ CFU/mL, which is ˜80% of the CFU/mL value obtained for the same cells grown on glucose (1.2×10⁹ CFU/mL). It is important to note that CFU/mL measurements likely underestimate the actual number of cells that are present in the cell-biomass mixture as many of the cells are expected to be adhered to the biomass and thus not accessible for plating. Enzyme attachment to the cell wall is critical, as control strain TDA18, which only secretes the three enzymes, exhibited negligible growth when cultured with pretreated corn stover and its CFU/mL value did not increase over time. To determine if all of the three enzymes are needed for robust bacterial growth, cells displaying either one or two enzymes were tested for their ability to grow in minimal media containing dilute acid pretreated corn stover. Strains containing all possible combinations of enzymes were examined (Table 6). Each strain contained the srtA and scaf genes integrated into the chromosome, as well as a plasmid that expresses either one or two of the cellulase-dockerin fusion proteins. As shown in FIG. 3, panel C, strains displaying two types of enzymes grew poorly when compared to strain TDA17, which contains the complete minicellulosome (strain TDA14 (Cel9E+Cel48F), open triangles; strain TDA15 (Cel5A+Cel9E), grey triangles; strain TDA16 (Cel5A+Cel48F), solid triangles). In particular, even after 72 hours their cultures reached OD₆₀₀ values of only ˜0.5-1.0, as compared to ˜2.5 for strain TDA18 (compare FIG. 3, panels A and C). Moreover, strains displaying only one type of cellulase exhibited negligible growth when cultured with pretreated biomass (FIG. 3, panel C).

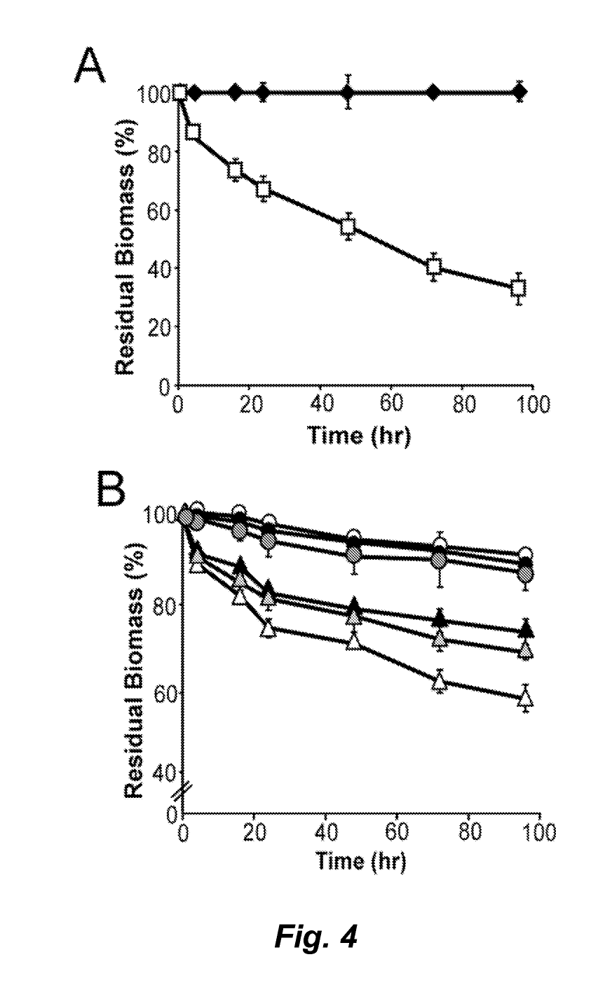

To quantitatively determine the amount of dilute acid pretreated corn stover degraded by the cells, we exposed the biomass to TDA17 cells that were defective in sugar import. TDA17 cells induced to express the minicellulosome were grown to saturation in rich media, killed by adding azide (0.1%), washed, and incubated with pretreated biomass. FIG. 4, panel A displays the percentage of biomass that was solubilized by the cells, which was determined by measuring the dry-weight of the corn stover before and after incubating with TDA17. Consistent with the growth data, TDA17 cells displaying all three enzymes solubilize the largest amount of pretreated biomass (62.3±2.6% of the biomass in 4 days). Strain TDA18, which only secretes the three cellulases, degraded only a small amount of the corn stover (solid diamonds). This makes sense as the cells are washed prior to exposure to biomass, and no enzymes are expected to remain on their cell surface. To determine the importance of each type of enzyme in biomass degradation, the cellulolytic ability of cells displaying only one or two types of enzymes was measured (FIG. 4, panel B). After 4 days, cells displaying two enzymes solubilized only 20-40% of the biomass (strain TDA14 (Cel9E+Cel48F), open triangles; strain TDA15 (Cel5A+Cel9E), grey triangles; strain TDA16 (Cel5A+Cel48F), solid triangles). In addition, strains displaying only one type of enzyme were unable to degrade corn stover to any significant degree (FIG. 4, panel B).

Measurement of Sugars Released from Dilute Acid Pretreated Corn Stover.

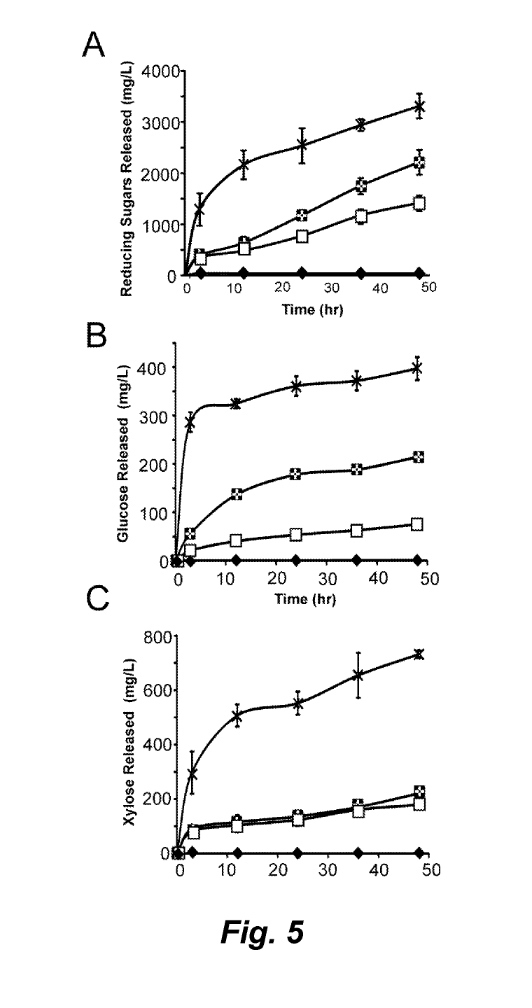

The amount of soluble reducing sugars (as well as glucose and xylose) liberated from dilute acid pretreated corn stover by azide-treated TDA17 cells was measured to further characterize their capacity to degrade biomass. As shown in FIG. 5, panel A, azide-treated cells displaying an intact minicellulosome liberated increasing amounts of reducing sugars over time, whereas few reducing sugars were released by enzyme secreting cells that were washed prior to biomass exposure. Interestingly, although the cells only display three types of enzymes, after 48 hours of exposure to the biomass they exhibit ˜30% of the activity of the CTec2/HTec2 enzyme cocktail produced by Novozymes Inc. (FIG. 5, panel A). For this comparison, conditions were chosen such that there was effective concentration of 15 mg of enzyme per gram dry weight biomass (as recommended by the manufacturer a mixture containing 13.5 mg CTec2 and 1.5 mg of HTec2 was used).

Corn stover is comprised of ˜36% glucose and ˜21% xylose, which reside within its cellulose and hemicellulose components, respectively (46). An analysis of the sugar content of the biomass before and after exposure to dilute acid revealed that pretreatment solubilized only a small fraction of the available sugars; 2% and 12% of the glucose and xylose were solubilized by dilute acid pretreatment, respectively (data not shown). After 48 hours the cells liberated 5% and 33% of the total available glucose and xylose in the biomass, respectively. Interestingly, the cells released ˜4 times more xylose than glucose, even though the pretreated biomass is primarily glucan (compare FIG. 5, panels B and C). This is consistent with cellulose cleavage by the cells generating cellodextrin polymers (cellobiose, cellotriose, cellotetraose, etc.) instead of monomeric glucose. This may also explain why the enzyme cocktails are capable of producing 6- and 5-fold more glucose and xylose, respectively, when they are incubated with the biomass as these mixtures presumably contain additional enzymes that are better suited for degradation of the soluble oligosaccharides into their component monosaccharides. To estimate the percent of glucan released as cellobiose, we measured the amount of glucose produced when the pretreated corn stover was incubated with intact cells supplemented with beta-glucosidase, an enzyme that hydrolyzes this disaccharide into glucose (FIG. 5, panel B). The addition of beta-glucosidase to the cell-biomass mixture resulted in a substantial increase in both glucose and reducing sugar production, but as expected did not alter the amount of xylose released by degradation of the hemicellulose (FIG. 5, panel C). In the presence of beta-glucosidase, the cells liberated 21% and 33% of the total available glucose and xylose in the biomass, respectively. The addition of beta-glucosidase, therefore, results in a 16% increase in the amount of glucose produced, indicating that the cells alone produce a substantial amount of cellobiose when they degrade the biomass (we estimate that cellobiose constitutes ˜36% of the sugars that are released from cellulose).

Growth on Untreated Corn Stover, Switchgrass, or Hatched Straw.

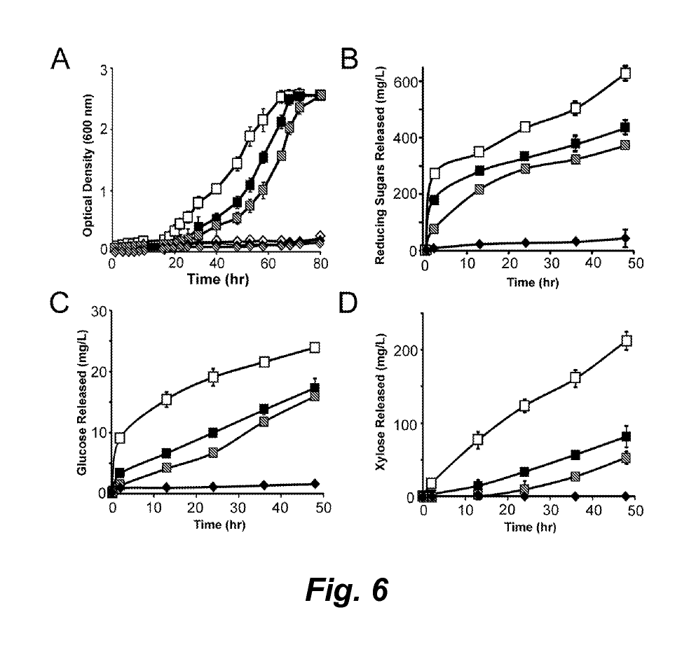

We cultured the minicellulosome displaying cells with various types of untreated biomass to determine whether thermochemical pretreatment was required for degradation. Cells grew to saturation in ˜60 hours when cultured in minimal media containing untreated corn stover, switchgrass, or hatched straw (FIG. 6, panel A). As with the pretreated biomass, only cells displaying all three enzymes grew robustly, and negligible growth occurred when the enzymes were secreted and not anchored to the cell wall. Importantly, the cells grew to similar densities as those grown with 0.5% w/v glucose as the sole carbon source. Interestingly, B. subtilis growth had distinct lag phases depending on the type of untreated biomass that was used as a nutrient source. The shortest lag occurred when cells were grown on corn stover, while longer lags were observed when straw or switchgrass was the sole carbon source. To compare the ability of cellulosome displaying cells to break down these different substrates, untreated biomass was exposed to azide-treated cells following the procedures described above. When untreated corn stover was degraded by azide-treated cells, 20% of the biomass was released as reducing sugars (FIG. 6, panel B), which included glucose (FIG. 6, panel C), xylose (FIG. 6, panel D), and other oligosaccharides. When switchgrass and straw were similarly degraded, they consistently released fewer reducing sugars, 12-15%, respectively (FIG. 6, panel B). As the cells were not supplemented with beta-glucosidase and only display three types of enzymes, it is likely that the biomass can be completely degraded by cells engineered to display larger, more elaborate minicellulosomes.

### Discussion

The production of ethanol and other commodities from sustainable biomass promises to reduce the world's dependency on petroleum. A major obstacle to its commercialization is the high cost of degrading biomass into fermentable sugars, which is typically achieved industrially through a two-step process in which the biomass is first thermochemically pretreated before it is degraded by adding cellulase enzymes (Himmel et al. (2007) Science 315: 804-807; Hendriks and Zeeman (2009) Bioresource Technol., 100: 10-18; Yeoman et al. (2010) Adv. Appl. Microbiol. 70: 1-55). In principle, major reductions in cost and gains in efficiency could be achieved by using bacteria to degrade the biomass instead of enzyme cocktails. One promising approach to achieve this objective is to create cellulolytic microbes that display multi-cellulase containing minicellulosomes. This has now been accomplished in S. cerevisiae and B. subtilis (Wilson (2011) Curr. Opin. Microbiol., 14: 259-263; Anderson et al. (2011) Appl. Environ. Microbiol., 77: 4849-4858; You et al. (2012) Appl. Environ. Microbiol., 78: 1437-1444; Tsai et al. (2009) Appl. Environ. Microbiol., 75: 6087-6093). While these microbes are capable of degrading amorphous purified cellulose and soluble cellulose (e.g., CMC), in some ex vivo assembly is required making them impractical for industrial purposes and it remains unknown whether they can grow using bona fide biomass as a primary nutrient source. Moreover, their cellulolytic activity has not been rigorously investigated. Here we report the construction of B. subtilis cells that display a minicellulosome that assembles without experimenter intervention. We demonstrate that the cells can degrade untreated biomass and use it as a nutrient source to grow.

To construct cells that display a self-assembled minicellulosome that could degrade biomass, we substantially redesigned our sortase-mediated protein display system, which we have shown is capable of displaying ex vivo assembled minicellulosomes on the surface B. subtilis (Anderson et al. (2011) Appl. Environ. Microbiol., 77: 4849-4858). Two major changes were made. First, we reengineered the cells to co-express all four components of the minicellulosome (previously, only the scaffoldin was expressed). This was achieved by expressing five genes: the sortase from B. anthracis (SrtA), a chimeric scaffoldin (Scaf) composed of three cohesin modules, and three dockerin-cellulase fusion proteins (Cel5A, Cel9E, and Cel48F) (FIG. 1A). Second, a different set of enzymes was incorporated into the minicellulosome. In particular, we displayed the Cel5A, Cel9E, and Cel48F enzymes because they are highly abundant in cellulosomes isolated from C. cellulolyticum cells cultured on wheat straw, and because they have been demonstrated to degrade biomass when present in purified minicellulosomes (Fierobe et al. (2005) J. Biol. Chem., 280: 16325-16334; Blouzard et al. (2010) Proteomics, 10: 541-554). In addition, Cel5A and Cel9E are bifunctional and therefore may reduce the total number of enzymes needed to be displayed in order to degrade biomass; Cel5A is both an endoglucanase and xylanase and Cel9E is an endoglucanase/exoglucanase (Id.). Finally, the enzymes are more likely to have optimal activities at the temperatures used to culture B. subtilis as they are derived from C. cellulolyticum, which is mesophilic. Based on our previous work, we anticipated that Scaf would be covalently attached to the cell wall cross-bridge peptide by SrtA, and that it would in turn non-covalently bind to the cellulase enzymes via dockerin-cohesin interactions. This is substantiated by our cell fractionation and immunoblot experiments that showed that each enzyme interacted with the appropriate cohesin domain within Scaf via its dockerin module (FIG. 1B). Interestingly, only ˜50% of each expressed cellulase was incorporated into the minicellulosome, while the rest of the protein was secreted. As our data shows that Scaf is completely saturated with cellulase enzymes, it is possible that some of the enzymes may not have properly refolded after crossing the membrane and thus be unable to bind Scaf, or that extracellular proteases secreted by B. subtilis removed the dockerin domains of the cellulases thereby rendering them unable to bind Scaf.

The self-assembled minicellulosomes enabled B. subtilis to grow robustly when dilute acid pretreated biomass was provided as a nutrient. Cells cultured with dilute acid pretreated corn stover approached similar CFU/mL values as those grown in soluble glucose (FIG. 3, panel B). The enzymes need to be displayed on the bacterial surface in order to facilitate growth, as cells that only secreted the enzymes grew poorly (FIG. 3, panel A). This demonstrates the importance of consolidating the enzymes into a minicellulosome for robust growth and degradation of biomass. Clustering the cellulases into a surface attached complex presumably enables them to function synergistically. In addition, the CBM modules within the complex enable the microbe to adhere to the biomass such that the resultant enzymatic degradation products are efficiently imported into the cell. Product import by the cell may also increase the effective activity of the enzymes, as it should reduce the concentration of cellobiose, which is known to act as a competitive inhibitor of the exoglucanase enzyme (Demain et al. (2005) MMBR 69: 124-154). Interestingly, cells grown on dilute acid pretreated corn stover had a significantly longer lag phase than cells grown on glucose (24-30 hours versus 4 hours, respectively) (FIG. 3, panels A and B). This presumably occurs because the endoglucanase activity of the minicellulosomes initially generates glucan polymers that are too large to be imported, resulting in delayed growth until the sugar polymers are further degraded by the exoglucanase to generate importable cellobiose and sugar monomers. This may be a common feature of naturally occurring cellulolytic bacteria as long lag phases are also observed when C. thermocellum is cultured with dilute acid pretreated corn stover (˜10 hour doubling time); a long lag phase may also be due to the self-catalyzing nature of C. thermocellum which can only grow as fast as the amount of cellulases it produces (Lynd et al. (1989) Appl. Environ. Microbiol., 55: 3131-3139).

Significantly, the minicellulosome displaying cells grew on three industrially relevant forms of biomass that did not require pretreatment with dilute acid: corn stover, hatched straw, and switchgrass. In all cases, after a significant lag, the cells achieved densities similar to those grown on glucose, and growth required that the cells display the enzymes on their surface (FIG. 6, panel A). The accessibility of the cellulosic fraction of the biomass to enzyme degradation appears to influence the length of the lag phase during growth. This is evident by the longer lag times associated with growth on untreated corn stover versus dilute acid pretreated corn stover (24 versus 16 hours, respectively). Moreover, it is supported by the growth data on the three different types of untreated biomass, which shows a negative correlation between the lignin content and growth rate (2); cells grew slowest on switchgrass, which contains the most lignin (22% lignin, 40 hour lag), and grew fastest on corn stover (15% lignin, 24 hour lag), which contains the least lignin (Garlock et al. (2009) Biotechnology for Biofuels, 2: 29; Kim et al. (2011) Bioresource Technol., 102: 11089-11096).

Cellulase mixtures used in industry to degrade biomass contain as many as sixty distinct enzymes and can completely hydrolyze the cellulosic and hemicellulosic components of pretreated lignocellulose within 24 to 48 hours (Banerjee et al. (2010) Biotechnol. Bioengineer., 106: 707-720). In order to benchmark our recombinant cells against these enzyme mixtures, we quantified the amount of sugar released from both untreated and dilute acid pretreated corn stover following exposure to azide-killed cells. In these studies, the conditions were chosen such that 15 mg of total cellulase enzymes were exposed to 1 g of biomass. This cellulase:biomass ratio is identical to that used by Walton and colleagues to study biomass degradation using enzyme mixtures and assumes that ˜150,000 minicellulosomes are displayed on each cell. This number was calculated by measuring the cellulolytic activity of Cel5A that has been bound to cells displaying Scaf. Consistent with the growth data, after washing, only cells that displayed a minicellulosome released significant amounts of oligosaccharides from both untreated and dilute acid pretreated corn stover. Moreover, the cellulolytic activity of the azide-killed cells was stable for at least 48 hours (FIGS. 4 and 5). As compared to a CTec2/HTec2 enzyme cocktail produced by Novozymes Inc. the cells are less active, which is not surprising as they display only three types of enzymes whereas this enzyme cocktail contains at least 20 different enzymes.

An analysis of the sugar content of dilute acid pretreated corn stover before and after cell exposure indicates that 21% of total glucan and 33% of the xylan is digested into its component monosaccharides by the cells (FIG. 5). This is consistent with the general structure of hemicellulose, which is amorphous and therefore more accessible to enzymatic hydrolysis. Presumably, the xylan is degraded by Cel5A which has xylanase activity (Fierobe et al. (2005) J. Biol. Chem., 280: 16325-16334; Zhang and Lynd (2003) Analyt. Chem., 75: 219-227), which agrees with similar studies that show that after 48 hours the azide-killed cells degraded 15% and 20% of the total glucan and xylan in untreated corn stover, respectively. It has been shown that six core enzyme activities are needed to efficiently hydrolyze biomass: endoglucanase, reducing end-acting exoglucanase, non-reducing end-acting exoglucanase, endoxylanase, beta-glucosidase, and beta-xylosidase (Banerjee et al. (2010) Biotechnol. Bioengineer., 106: 707-720). Our minicellulosomes possess only three of these activities (endoglucanase, exoglucanase, and xylanase), which likely explains why our cells did not completely degrade the biomass. This notion is consistent with our studies of azide-killed cells, which demonstrated that the cells do not completely digest the cellulose component of the biomass into glucose, but instead produce cellobiose and other cellodextrins. Currently, we are working to expand the number and types of enzymes present in the minicellulosome to more completely degrade glucan and xylan polymers, which should greatly increase the cellulolytic activity of the cells. To guide the construction of these more elaborate complexes will require more rigorous quantitative saccharification methods to be used to determine the specific breakdown products that are generated by the cells (Zhang and Lynd (2003) Analyt. Chem., 75: 219-227).

Recently, several model organisms have been engineered to grow on pretreated biomass. However, to the best of our knowledge, the B. subtilis strain reported herein is the first recombinant bacterium that has been demonstrated to have the ability to grow on untreated biomass. While native strains of B. subtilis can potentially subsist on untreated plant biomass, the laboratory strains created in this study could only grow on untreated biomass when functional minicellulosomes were displayed. The robustness of our recombinant B. subtilis cells was likely due to the sortase-mediated attachment system that allowed high copy-number display of the minicellulosome without the need for ex vivo assembly. In addition, unlike other previously described systems, the minicellulosomes are covalently anchored to the peptidoglycan, and thus presumably more stable and better suited for industrial applications (Lilly et al. (2009) FEMS Yeast Res., 9: 1236-1249; You et al. (2012) Appl. Environ. Microbiol., 78: 1437-1444; Tsai et al. (2009) Appl. Environ. Microbiol., 75: 6087-6093). Biofuels and many other high-value bio-based chemicals and materials can be produced from only twelve biomass-derived building blocks (Reddy and Yang (2005) Trends in Bbiotechnology 23: 22-27; Werpy et al. (2004) Top Value Added Chemicals From Biomass. Volume 1-Results of Screening for Potential Candidates From Sugars and Synthesis Gas. DTIC Institution). B. subtilis shows great promise for producing several of these compounds, since unlike many other currently used industrial microbes, it naturally imports and metabolizes cellobiose and C5 sugars (Tobisch et al. (1997) J. Bacteriol., 179: 496-506; Stulke and Hillen (2000) Ann. Rev. Microbiol., 54: 849-880). Moreover, using its robust genetic system, several investigators have already introduced into B. subtilis the relevant metabolic pathways needed to produce some of these compounds (Tobisch et al. (1997) J. Bacteriol., 179: 496-506; Schallmey et al. (2004) Canadian J. Microbiol., 50: 1-17; Li et al. (2011) Appl. Microbiol. Biotechnol., 91: 577-589; Xue and Ahring (2011) Appl. Environ. Microbiol., 77: 2399-2405; Romero et al. (2007) Environ. Microbiol., 73: 5190-5198), which, when paired with the cellulose degrading system we have created, enables the direct production of many valuable biocommodities from biomass.

### Example 2

### Enhancing Lignocellulose Degradation by Rationally Enlarging the Cell Surface Displayed Designer Cellulosome

### Introduction

Dwindling supplies of petroleum have encouraged the production of renewable biofuels from lignocellulosic biomass (Kerr (2008) Science, 322: 1178-1179). However, a significant problem deterring the enhanced use of fuels derived from lignocellulose is the cost of the cellulase enzyme cocktails that are used to degrade the plant biomass into fermentable sugars (Banerjee et al. (2010) Biotechnol. Bioeng. 106: 707-720; Banerjee et al. (2010) Bioresour. Technol. 101: 9097-9105). An alternative to the use of purified cocktails to hydrolyze lignocellulose includes the construction of recombinant cellulolytic microbes that display cellulase enzymes (la Grange et al. (2010) Appl. Microbiol. Biotechnol. 87: 1195-1208). Microbes that display multi-cellulase complexes generally experience high degrees of synergy between the displayed enzymes and can effectively enable the microorganism to adhere to the biomass which make them an attractive replacement of the cellulase cocktails (Fontes and Gilbert (2010) Ann. Rev. Biochem., 79: 655-681). Due to the promising applications of cellulosomes in industrial lignocellulose degradation, several research groups have displayed designer cellulosomes on the surface of Saccharomyces cerevisiae and Bacillus subtilis (Anderson et al. (2013) Appl. Environ. Microbiol. 79: 867-876; Anderson et al. (2011) Appl. Environ. Microbiol. 77: 4849-4858; Fan et al. (2012) Proc. Natl. Acad. Sci. USA, 109: 13260-13265; Goyal et al. (2011) Microb. Cell Fact., 10: 89; Kim et al. (2013) Microb. Cell. Fact. 12: 14; Tsai et al. (2009) Appl. Environ. Microbiol. 75: 6087 ˜6093; Tsai et al. (2010) Appl. Environ. Microbiol. 76: 7514-7520; Wen et al. (2010) Appl. Environ. Microbiol. 76: 1251-1260; You et al. (2012) Appl. Environ. Microbiol. 78: 1437-1444).

Though displaying the Clostridium cellulolyticum cellulases Cel5A, Cel9E and Cel48F in a designer cellulosome on the B. subtilis cell surface enabled these cells to efficiently degrade lignocellulose, the total amounts of reducing sugars released were three to six fold lower than commercially available cocktails, indicating that certain enzyme activities could be missing or that the absolute number of enzymes was too low (Anderson et al. (2013) Appl. Environ. Microbiol. 79: 867-876). This hypothesis can be supported by the notion that essential activities such as xylanases were missing in the surface displayed cellulosome and that the commercial cellulase cocktails contain up to eighty different enzymes, while the cell-displayed designer cellulosomes consisted of only three (Anderson et al. (2013) Appl. Environ. Microbiol. 79: 867-876; Banerjee et al. (2010) Biotechnol. Bioeng. 106: 707-720; Banerjee et al. (2010) Bioresour. Technol. 101: 9097-9105). Therefore, it became apparent that perhaps both of these issues could be resolved by increasing the number of enzymes displayed whose activities are different, yet complementary, to what is already present.

The choice of cellulase and hemicellulase enzymes to incorporate into the designer cellulosome is critical for obtaining optimal lignocellulose degradation. Work has been published using proteomics approaches to determine what cellulases are most prominently displayed in naturally assembled cellulosomes (Blouzard et al. (2010) Proteomics, 10: 541-554; Fendri et al. (2009) FEBS J., 276: 3076-3086; Raman et al. (2009) PloS One 4: e5271). Some of these studies were performed by culturing the bacteria on model substrates such as cellobiose, regenerated amorphous cellulose (RAC), and phosphoric acid swollen cellulose (PASC) (Blouzard et al. (2010) Proteomics, 10: 541-554). After culturing, the bacteria were collected and the purified cellulosomes subjected to mass spectrometry to identify and characterize the abundance of each protein found within the cellulosome. At least two dozen enzymes were found to be prominent in the isolated cellulosomes, but because the bacteria were not cultured on lignocellulosic substrates, these proteomic profiles may not necessarily reflect the composition of the complex when they are cultured on plant-based biomass. Therefore, in order to determine how the cellulosomal composition of the C. thermocellum and C. cellulolyticum cellulosomes changes when exposed to industrially relevant forms of biomass, these bacteria were cultured on switchgrass and straw, respectively (Fendri et al. (2009) FEBSJ., 276: 3076-3086; Raman et al. (2009) PloS One 4: e5271). Interestingly, it was observed that glycoside hydrolase (GH) family 9 and 48 enzymes appear to be particularly enriched in cultures that have been grown on pretreated lignocellulose (Fendri et al. (2009) FEBSJ., 276: 3076-3086). This may be due to a possible enhanced synergy between enzymes of these families. In addition, it has been observed that purified designer cellulosomes containing combinations of the C. cellulolyticum enzymes Cel9G paired with Cel9E or Cel48F experience at least a seven fold enhancement on crystalline cellulose (Fierobe et al. (2001) J. Biol. Chem. 276: 21257-21261; Fierobe et al. (2005) J. Biol. Chem. 280: 16325-16334; Mingardon et al. (2007) Appl. Environ. Microbiol. 73: 7138-7149). Family5 GH enzymes were also highly abundant and included the Man5A mannanase that could be crucial for efficient degradation of the hemicellulose component (Fendri et al. (2009) FEES J., 276: 3076-3086; Raman et al. (2009) PloS One 4: e5271). The interesting conundrum with Man5A is that it has also been found to be abundant when the bacteria were cultured on crystalline cellulose as opposed to lignocellulose, though it is not clear why this may be happening (Raman et al. (2009) PloS One 4: e5271). This phenomenon has also been observed with other hemicellulase enzymes and demonstrates that a global view of cellulosome composition does not necessarily indicate why some enzymes are present (Fendri et al. (2009) FEES J., 276: 3076-3086; Raman et al. (2009) PloS One 4: e5271).

Based on inter alia results described above, lignocellulose degradation can potentially be improved by the expansion of the surface-displayed cellulosome to include more than three enzymes. One goal of this work is to expand the cellulosome to as many as nine enzymes in order to resemble the C. thermocellum cellulosome. To determine the feasibility of efficiently displaying a cellulosome containing nine different enzymes, a smaller, more manageable complex containing six different cellulases was constructed. In addition to Cel5A, Cel9E and Cel48F which have previously been displayed, the C. thermocellum enzymes XynA (endoxylanase), Man5A (mannanase) and CelS (exoglucanase) were chosen to be displayed based on proteomics data showing their high abundance in cellulosomes isolated from C. thermocellum cultured on switchgrass (Raman et al. (2009) PloS One 4: e5271). Strains displaying six unique enzymes proved to be more effective at degrading both dilute-acid pretreated and untreated lignocellulosic substrates. This increased efficiency enabled the cells to reach similar degradation capacity as purified cellulase cocktails and permitted faster growth on lignocellulosic substrates. This is an important demonstration in the feasibility of enlarging the cellulosome to display more than three enzymes, and can be used as a model for the creation of strains displaying even larger cellulosome complexes.

### Materials and Methods

Construction of B. subtilis Strains.

Strains of B. subtilis were created that could assemble a six-enzyme containing designer cellulosome (FIG. 7A). Descriptions of strains and plasmids constructed can be found in Table 7. To plasmid pBL112 the B. anthracis srtA gene was cloned into the HindIII and SpeI sites via PCR amplification of srtA and by restriction digestion and ligation with T4 DNA ligase to create plasmid pSrtA (Lanigan-Gerdes et al. (2007) Molecular Microbiol., 65: 1321-1333). In order to append the type-II cohesin (coh-II) module to Scaf-I, coh-II was amplified from the C. thermocellum genome, restriction digested with NotI, and ligated into plasmid pScaf, and created plasmid pScaf-I. scaf-I with the appended coh-II and the C-terminal portion of fibronectin binding protein B (that contained the cell wall sorting signal) was then PCR amplified and restriction digested with SpeI and SphI to enable ligation into pSrtA, generating pSrtA/Scaf-I. To generate the Scaf-II construct, scaf was subcloned into pET28a into the NheI and XhoI sites. The type-II dockerin (doc-II) module from cipA was amplified from the C. thermocellum genome, restriction digested with XhoI and SphI and ligated into this plasmid. The following fused gene was PCR amplified and restriction digested with SphI and cloned into pSrtA/Scaf-I to generate pSrtA/Scaf-I/Scaf-II. This plasmid encodes the SrtA transpeptidase; Scaf-I contains the B. subtilis PhrC secretion signal, scaffoldin domain, type-II cohesin module and C-terminal domain of fibronectin binding protein B; and Scaf-II contains the B. subtilis PhrC secretion signal, the same scaffoldin domain as in Scaf-I and the type-II dockerin module from C. thermocellum CipA. These genes are inducible upon addition of IPTG under the control of the P_{spac} promoter. The C. cellulolyticum genes cel5A, cel9E, and cel48F have been cloned into the B. subtilis expression plasmid pHCMC05 and have been described previously (Anderson et al. (2013) Appl. Environ. Microbiol. 79: 867-876). The genes encoding CelS, XynA and Man5A were cloned into the B. subtilis expression plasmid pDG148 (Stragier et al. (1988) Cell, 52: 697-704). celS (with its native dockerin sequence) was PCR amplified from the C. thermocellum genome and ligated into the SwaI site within pDG148. xynA (lacking its dockerin sequence) from C. thermocellum, was appended with the type-I dockerin from C. cellulolyticum and cloned into the AscI and SpeI sites within pDG148. Finally, man5A from C. thermocellum (lacking its native dockerin) was appended with the type-I dockerin sequence from R. flavefaciens and cloned into the SpeI site in pDG148. To each gene the phrC secretion signal and a hexahisitidine tag were appended to the 5′ end of the gene to ensure proper secretion and detection of the proteins encoded by the genes. The final plasmid generated was then called pSXM and is inducible by IPTG due to the presence of the P_{spac} promoter. B. subtilis strains (derived from the wprA⁻ strain BAL2238) containing pSrtA/Scaf-I/Scaf-II and/or pCellulase/pSXM were generated by transforming strain BAL2238 with the corresponding plasmids using standard methods and were selected on Luria Bertani (LB) agar plates containing 1 μg/ml erythromycin, 5 μg/ml chloramphenicol and/or 5 μg/ml neomycin (Anderson et al. (2011) Appl. Environ. Microbiol. 77: 4849-4858).

TABLE 7
Bacillus subtilis strains and plasmids used in this study.

| | | | Reference |
|---|---|---|---|
| Strain | Relevant genotype | Phenotype^{a} | or Source |
| BAL2238 | wprA::hyg trpC2 pheA1 | | 1 |
| TDA10 | pSrtA/Scaf | SrtA, Scaf^{b,c} | 1 |
| TDA17 | PSrtA/Scaf; pCellulase | SrtA, Scaf, Cel5A, Cel9E, | 1 |
| | | Cel48F^{b,c,d} | |
| TDA18 | pScaf | Scaf^{b} | 1 |
| TDA20 | pCellulase; pSXM | Cel5A, Cel9E, Cel48F, CelS, | 1 |
| | | Man5A, XynA^{d,f} | |
| TDA21 | pSrtA/Scaf-I/Scaf-II | SrtA, Scaf-I, Scaf-II^{b,e} | This study |
| TDA22 | pSrtAScaf-I/Scaf-II; | SrtA, Scaf-I, Scaf-II, Cel5A, | This study |
| | pCellulase; pSXM | Cel9E, Cel48F, CelS, Man5A, | |
| | | XynA^{b,d,e,f} | |

| | | Reference |
|---|---|---|
| Plasmid | Relevant Characteristics^{g} | or Source |
| pBL112 | B. subtilis-E. coli shuttle plasmid that integrates into thrC | 27 |
| | locus in the B. subtilis genome, IPTG inducible promoter, | |
| | Amp^{r}, Ery^{r} | |
| pHCMC05 | B. subtilis expression plasmid with IPTG inducible promoter, | BCSC^{h} |
| | Amp^{r}, Cm^{r} | |
| pDG148 | B. subtilis expression plasmid with IPTG inducible promoter, | 37 |
| | Amp^{r}, Neo^{r} | |
| pSrtA/Scaf | B. anthracis srtA and scaf in pBL112 | 1 |
| pScaf | B. anthracis srtA and scaf in pBL112 | 1 |
| pSrtA/Scaf- | B. anthracis srtA, scaf-I, and scaf-II in pBL112 | This study |
| 1/Scaf-II | | |
| pCellulase | C. cellulolyticum cel5A, cel9E, cel48F in pHCMC05 | 1 |
| pXSM | C. thermocellum celS, man5A, xynA in pDG148 | This Study |

^{a}Protein(s) expressed by the strains.
^{b}Flag-tagged full length sortaseA from B. anthracis strain Ames
^{c}Scaf contains three type I cohesin domains from C. thermocellum, C. cellulolyticum, and R. flavefaciens, a CBM, and the sorting signal from Staphylococcus aureus fibronectin binding protein B and a HA epitope tag.
^{d}C. cellulolyticum Cel5A appended with a His₆ tag, C. cellulolyticum Cel9E appended with a VSV-G epitope tag and C. cellulolyticum Cle48F appended with a Myc epitope tag.
^{e}Scaf was appended with a type-ll cohesin from C. thermocellum immediately before the sorting signal domain, creating Scaf-I. Scaf-ll was generated by removing the sorting signal domain and replacing it with a type-ll dockerin from C. thermocellum
^{f}C. thermocellum CelS, C. thermocellum Man5A, C. thermocellum XynA. All proteins were appended with a His₆ tag.
^{g}Amp^{r}, ampicillin resistance; Ery^{r}, erythromycin resistance; Cm^{r}, chloramphenicol resistance; Neo^{r}, neomycin resistance.
^{h}Bacillus Genetic Stock Center

Cell Fractionation and Immunoblot Analysis of Self-Assembled Cellulosome.

B. subtilis cells were cultured overnight in LB media supplemented with 5 μg/ml chloramphenicol and 5 μg/ml neomycin. A 500 μl aliquot of the overnight culture was then used to inoculate 50 ml of fresh LB media supplemented with the same antibiotics. After the cultures reached an optical density at 600 nm (OD₆₀₀) of 0.1, 1M IPTG was added to a final concentration of 1 mM to induce protein expression. After the cells have reached saturation, generally after 4 h, the cells were collected by centrifuging at 3,000×g for 5 min, and the cell pellet resuspended in 1 ml STM buffer (25% sucrose, 50 mM Tris-HCl pH 8.0, 5 mM MgCl₂). Following resuspension, the cells were centrifuged at 3,000×g for 5 min, and the pellet again resuspended in STM buffer such that the cell densities between cultures was the same (OD₆₀₀ ˜10). The cells were then subjected to lysozyme treatment by the addition of 500 μg/ml lysozyme, and the cell walls solubilized by incubation at 37° C. for 30 min. The suspension was then centrifuged at 20,000×g for 10 min and the solubilized cell walls (found in the supernatant) were collected and stored at −20° C. The solubilized proteins found within the cell wall fraction were then analyzed by immunoblot, and has been described elsewhere (Anderson et al. (2013) Appl. Environ. Microbiol. 79: 867-876).

Determination of the Levels of Scaf-I/Scaf-II Displayed on the Cell Surface.

For these studies, full-length Cel5A was expressed and purified from E. coli. Cel5A was subcloned into a pET28a plasmid. After transformation into E. coli BL21 (DE3) cells, the protein was expressed using standard procedures at 18° C. overnight. Following protein expression, the cell pellet was resuspended in lysis buffer (25 mM Tris-HCl, pH 7.0, 250 mM NaCl, 25 mM CaCl₂) and sonicated; the supernatant was collected by centrifugation at 20,000×g for 30 min. The supernatant was then passed through HisPur (Pierce, Inc.) cobalt-nitrilotriacetic acid (Co-NTA) resin, washed with 10 column volumes of lysis buffer supplemented with 10 mM imidazole, and eluted in lysis buffer supplemented with 150 mM imidazole. The proteins were then buffer exchanged into binding buffer (20 mM Tris-HCl, pH 6.0, 2 mM CaCl₂) and concentrated, and the amount of protein was quantified using a bicinchoninic acid (BCA) assay.

In order to determine the amount of Scaf-I and Scaf-II displayed on the cell surface, increasing amounts of purified Cel5A was incubated with cells expressing and displaying Scaf or Scaf-I and Scaf-II. To 50 ml of fresh LB media, an inoculant containing 500 μl of an overnight culture was added and the culture induced to express Scaf or Scaf-I/Scaf-II for 4 hr. After the cells reached saturation, they were collected by centrifugation at 3,000×g for 10 min. To prevent non-bound Scaf or Scaf-I/Scaf-II from interfering in the assay, the cells were resuspended in 1 ml binding buffer and centrifuged at 3,000×g for 5 min, and repeated for a total of three times. To a total of 2.4×10¹⁰ cells used for each assay, an increasing amount of Cel5A was added, and allowed to incubate with the cells. Following incubation on ice for 1 hr, the cells were collected by centrifugation at 3,000×g for 5 min, resuspended in 1 ml binding buffer and centrifuged again at 3,000×g for a total of three times. Following the third wash, the cells were resuspended in 1 ml 0.5% carboxymethyl cellulose (CMC) dissolved in assay buffer (20 mM Tris-Acetate pH 6.0, 2 mM CaCl₂) and incubated at 37° C. for 1 hr. Following incubation, the suspension was centrifuged and the reducing sugars found in the supernatant analyzed with the dinitrosalicylic acid assay solution (1% dinitrosalicylic acid, 1% NaOH, 0.2 phenol, 0.1 NaSO₃). The solution was boiled for 10 min and the amount of reducing sugars present determined by reading the absorbance at 575 nm. Glucose was used as a standard. Control strains in which SrtA was not present, preventing covalent attachment of the scaffoldin proteins, were used. The assays were performed in triplicate and the error represented is the standard deviation.

Studies of Bacterial Growth on Lignocellulosic Biomass.

Untreated corn stover, switchgrass, and hatched wheat straw were washed with deionized water and frozen at −20° C. For some assays, the corn stover was first pretreated using dilute sulfuric acid as described previously (Anderson et al. (2013) Appl. Environ. Microbiol. 79: 867-876; Jensen et al. (2010) Bioresource Technol., 101: 2317-2325). Following autoclaving, the suspension was neutralized by washing with deionized water and stored at −20° C. Strains displaying three and six cellulases (TDA17 and TDA22, respectively) were tested for their ability to grow on untreated and pretreated biomass. Colonies from agar plates were used to inoculate a 5 ml LB culture supplemented with 5 μg/ml chloramphenicol and/or 5 μg/ml neomycin in order to select for transformants that contain plasmid pCellulase and/or pSXM. After 8 h of growth at 37° C., 100 μl of each culture was transferred into 5 ml of M9 medium that contained 0.5% (wt/vol) glucose. The medium also contained 0.004% tryptophan, 0.004% phenylalanine, and 0.004% threonine, as the parent strain is auxotrophic for these amino acids. After 16 h of growth, 100 μl of each culture was used to inoculate a 5 ml culture that contained biomass as the sole carbon source. This medium consists of M9 minimal medium and 0.5% (wt/vol) pretreated/untreated biomass. In control experiments, the biomass was replaced with 0.5% (wt/vol) glucose. To induce protein expression, 1 mM IPTG was added immediately after inoculating the biomass-containing culture. The OD₆₀₀s of the cultures were measured over a 72 to 96 h period. Control strains in which no cellulases were displayed served as negative controls. Growth assays were performed in triplicate, and the errors represented are the standard deviation.

Whole-Cell and Cellulase Cocktail Sugar Release Assays.

Cells were induced for protein expression and were grown to saturation in LB medium as described above. They were then centrifuged at 3,000×g for 10 min, resuspended in assay buffer (20 mM Tris-acetate, pH 6.0, 1 mM CaCl2, 0.1% sodium azide), recentrifuged, and the final cell pellet was resuspended in assay buffer. Lignocellulosic biomass was then added to the cell suspension such that there was a total of ˜15 mg of cell-displayed cellulase enzymes per gram of biomass; 10 ml suspensions containing cells at an OD₆₀₀ of 2.5 were incubated with 50 mg of biomass at 37° C. with shaking. For the assays performed using commercially available cocktails, a mixture containing 13.5 mg of CTec2 and 1.5 mg of HTec2 enzyme cocktails (Novozymes Inc.) per gram of biomass was shaken in 10 ml of assay buffer at 37° C. To measure the amount of total biomass degraded, the cell-biomass mixture was removed at various times from the shaker and the insoluble biomass was allowed to settle. Measurement of reducing sugars released into the medium was accomplished as described previously and made use of dinitrosalicylic acid (glucose was used as the standard). Glucose was assessed using a glucose assay kit (Eton Biosciences) that makes use of the glucose oxidase enzyme, and the assay followed procedures outlined by the manufacturer. Xylose release was analyzed using phloroglucinol (Fisher) as described previously (Anderson et al. (2013) Appl. Environ. Microbiol. 79: 867-876). Control experiments made use of strain TDA19, which lacks scaf and srtA but contains the cellulase-expressing plasmids, resulting in cellulase secretion. Assays were performed in triplicate, and the standard deviation was used as an estimate of the error.

Dionex Analysis of Sugars Released by Cells Displaying Three or Six Enzymes and Cellulase Cocktails.

In order to identify the types of cellodextrins and xylodextrins solubilized by azide-killed cells displaying three or six enzymes and purified cellulase cocktails when degrading untreated corn stover, similar experiments were performed as described above. Samples from cells that had grown on the untreated corn stover were also collected in order to determine the degree of biomass solubilization during growth and the methods used are identical to those described above. After incubation of the untreated corn stover with cells displaying three or six enzymes or the cellulase cocktail, the samples were transferred to a glass culture tube and autoclaved on the liquid cycle for 10 min. Following autoclaving, the samples, including supernatant and insoluble material, was transferred to 15 ml plastic tubes and frozen at −80° C. Before shipping to our collaborator, Dr. Henri-Pierre Fierobe at CNRS in France, the tubes were placed in a styrofoam box and filled with dry ice for shipping.

The insoluble biomass was completely hydrolyzed using concentrated sulfuric acid and high temperature to release all remaining monosaccharides that were not solubilized by the cells or cellulase cocktail. To both the solubilized monosaccharides from the residual lignocellulose and the soluble carbohydrates released into the supernatant by the cell-displayed enzymes and cellulase cocktail, 200 μl samples were taken and analyzed using a Dionex PA-1 anion exchange HPLC column, and this procedure has been described elsewhere (Westereng et al. (2013) J. Chromatog. A 1271: 144-152; Widmer (2010) Biotechnol. Letts., 32: 435-438). The residual sugars and solubilized carbohydrates from three independent experiments were collected.

### Results and Discussion

B. subtilis Cells Display Enlarged Six-Enzyme Containing Designer Cellulosomes.

Recombinant B. subtilis displaying three cellulase enzymes (strain TDA17) enabled effective growth of the cells when lignocellulose was provided as the sole carbon source (Anderson et al. (2013) Appl. Environ. Microbiol. 79: 867-876). However, these strains were more than three-fold less active than commercially available cellulase cocktails on dilute acid-pretreated corn stover, and had a significant lag phase when cultured on plant biomass as a nutrient. This indicated that the numbers of enzymes and/or enzyme activities were lacking, and to enable for more efficient degradation and growth on lignocellulose, the cellulosome was enlarged to include more enzymes with complementary activities. In addition to the enzymes previously displayed, three new C. thermocellum enzymes: CelS (exoglucanase), XynA (endoxylanases), and Man5A (mannanase) were also expressed and displayed (FIG. 7A). CelS may be important in efficient saccharification because it is highly abundant in native cellulosomes isolated from C. thermocellum, and when genetically deleted, a significant growth defect when cultured on cellulose was observed (Olson et al. (2010) Proc. Natl. Acad. Sci. USA, 107: 17727-17732; Raman et al. (2009) PloS One 4: e5271; Wilson (2010) Proc. Natl. Acad. Sci. USA, 107:17855-17856). XynA is also quite abundant in native cellulosomes and has been characterized to act effectively on xylan and other hemicellulose components (Fernandes et al. (1999) Biochem. J. 342: 105-110; Raman et al. (2009) PloS One 4: e5271). Finally, Man5A has recently been characterized to be active on mannan and other hemicellulose carbohydrates and is also highly abundant in cellulosomes isolated from C. thermocellum grown on switchgrass, indicating that it may be essential in helping to degrade hemicellulose (Mizutani et al. (2012) Appl. Environ. Microbiol. 78: 4781-4787; Raman et al. (2009) PloS One 4: e5271). In addition to expanding the number of enzymes present in the surface displayed cellulosome, it also resembles the more complicated complexes of A. cellulolyticus and R. flavefaciens (FIG. 7A). The anchoring scaffoldin (Scaf-I) is similar to the Scaf scaffoldin described previously, and has been appended with the type-II cohesin module from C. thermocellum SdbA (Anderson et al. (2013) Appl. Environ. Microbiol. 79: 867-876). Scaf-II that is not covalently anchored to the cell wall contains the type-II dockerin module from C. thermocellum CipA to enable binding to Scaf-I. This is the first example of a recombinant organism that is capable of displaying six unique cellulase enzymes that spontaneously assemble into a surface-displayed cellulosome.

Western blotting analysis of cells expressing all six cellulase enzymes and the two scaffoldin proteins (strain TDA 22) indicate that the enzymes can correctly incorporate into the cellulosome as cells that lack the Scaf-I and Scaf-II scaffoldins (TDA20) cannot anchor the cellulase proteins to the cell wall (FIG. 7B). Interestingly, due to the degeneracy of the cohesin/dockerin module interactions used in the enhanced cellulosome, multiple compositions of the complex can potentially form. This is reminiscent of natural cellulosomes in which the cellulase enzymes can bind to any of the cohesin modules within the scaffoldin (Fontes and Gilbert (2010) Ann. Rev. Biochem., 79: 655-681). The ability of the cellulosomes displayed on the cell surface to contain different mixtures of enzymes (within cellulosomes localized on one cell, as well as the whole culture) may potentially be beneficial in lignocellulose degradation.

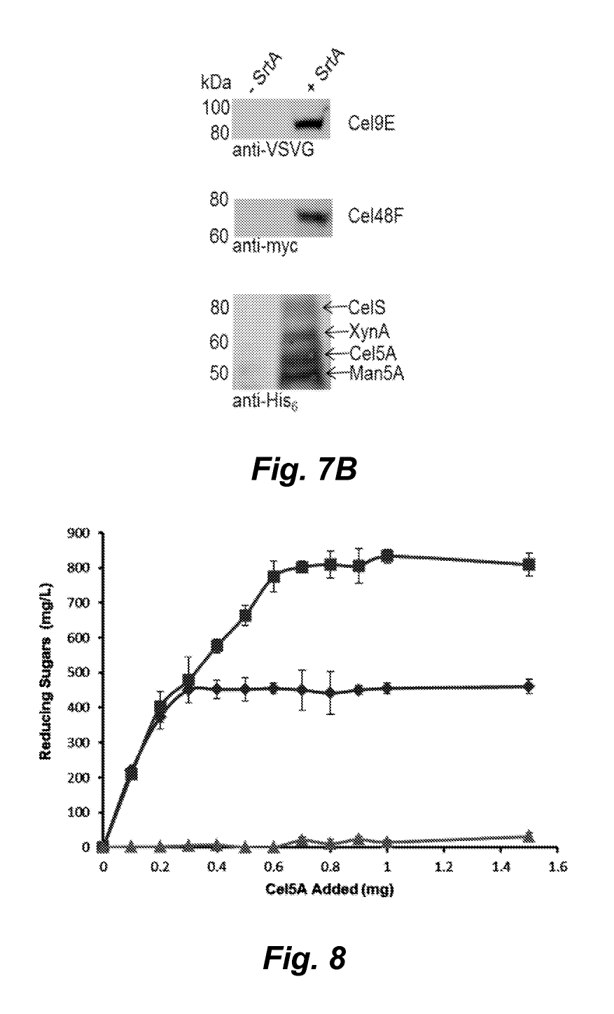

Cells of B. subtilis that displayed cellulosomes containing three enzymes averaged 150,000 complexes displayed per cell (Anderson et al. (2013) Appl. Environ. Microbiol. 79: 867-876). Similar experiments to determine the number of complexes displayed by strain TDA21 were performed, and nearly twice as much activity on CMC was observed, indicating that about twice as much Cel5A was displayed as compared to the strain displaying only a single scaffoldin (FIG. 8). This demonstrates that the two C. cellulolyticum cohesin modules available for binding have been occupied by Cel5A. Furthermore, it shows that expansion of the cellulosome to display more than three enzymes does not appear to hinder the ability to robustly display large complexes on the cell surface of B. subtilis. In the future, quantitative PCR experiments can be performed to more accurately monitor expression levels of each component to ensure proper expression. Finally, this data reveals that the type-II cohesin/dockerin pair incorporated into the extended cellulosome has interacted, suggesting that designing chimeric cellulosomes in this fashion is feasible and useful.

B. subtilis Cells Displaying Six Enzymes Enable Enhanced Growth on Dilute Acid Pretreated Corn Stover and Untreated Lignocellulosic Biomass Substrates.

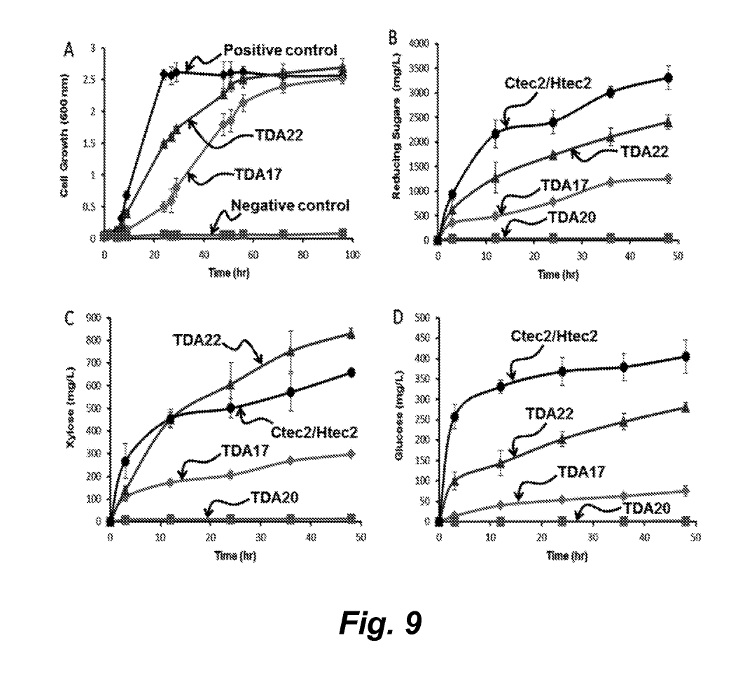

B. subtilis that displayed only Cel5A, Cel9E, and Cel48F were able to utilize dilute acid pretreated corn stover as a nutrient for growth. Though after 60 hours the cultures were able to reach similar densities as those cultured with glucose, a significant lag phase was observed (10-12 hours) before transition into exponential growth (Anderson et al. (2013) Appl. Environ. Microbiol. 79: 867-876). It was hypothesized that cells of strain TDA17 were initially unable to quickly degrade the lignocellulose into metabolizable soluble monosaccharides and dextrans, resulting in this lag phase, until enough cellulosome complexes were assembled that enabled more robust lignocellulose degradation. Increasing the number of enzymes would presumably improve the rate of degradation because the display of more enzymes should increase solubilization of the insoluble carbohydrates. Cells of strain TDA22 that displayed the six enzymes did show an increased growth rate, and the lag was reduced to ˜5 hours (FIG. 9, panel A, compare blue triangles (TDA22) and red diamonds (TDA17)). Due to the decreased lag phase, the cells were able to reach saturation after 42 hours (as opposed to 60 hours for the cells displaying three enzymes), indicating that it may be beneficial to include additional enzymes with complementary activities in order to obtain optimal digestion. Though the lag phase was decreased by 50%, TDA22 cells were not able to achieve the small lag observed when cells were cultured with glucose (˜2 hours). This indicates that additional cellulase enzymes may need to be incorporated in order to achieve similar growth characteristics as cells cultured with glucose.

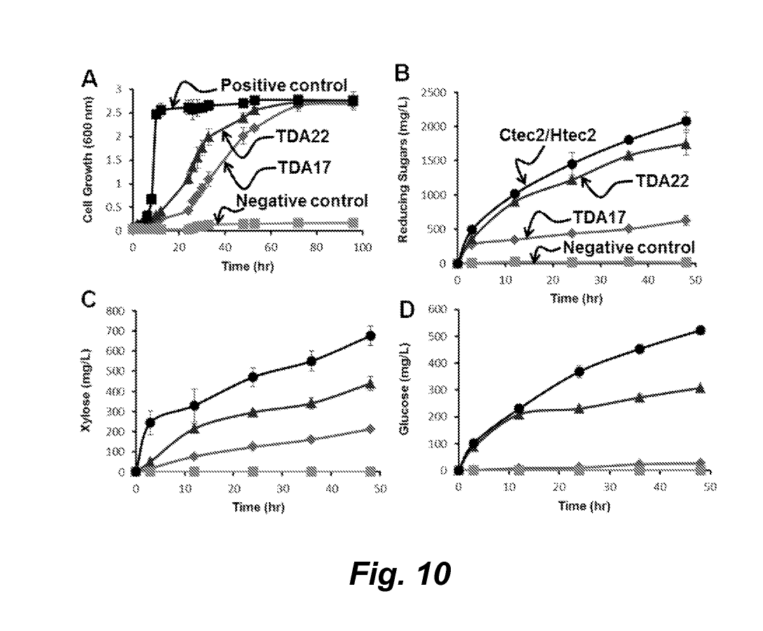

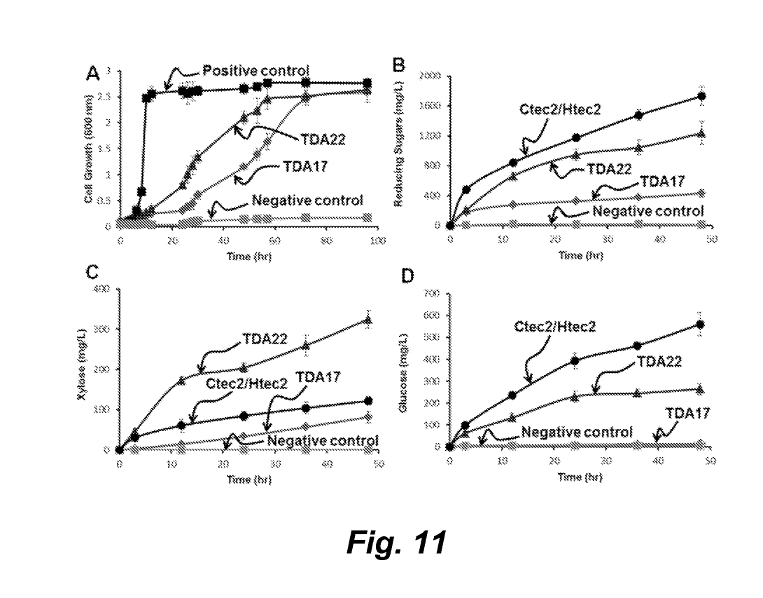

A similar pattern of growth was observed when cells displaying six cellulases were cultured on untreated corn stover, switchgrass and straw (FIG. 10, panel A, 11, panel A, and 12, panel A, triangles). The growth lag for untreated corn stover was decreased more than 46% (24 hours to 13 hours) while that for both straw and switchgrass were reduced by more than 22%. Interestingly, though better growth was observed on untreated corn stover by strain TDA22, the lag phase was still longer than those observed for strain TDA17 grown on pretreated biomass (compare FIG. 9, panel A with 10, panel A). The inability to reduce the lag phase as significantly when cultured on untreated substrates as compared to pretreated lignocellulose could be due to the additional hemicellulose present that reduces access to the cellulose. In addition, the slower growth on untreated straw and switchgrass as opposed to corn stover was also observed even when six cellulases were displayed could potentially be due to the higher amounts of lignin present in these substrates (FIGS. 11, panel A and 12, panel A) (Garlock et al. (2009) Biotechnology for Biofuels, 2: 29; Kim et al. (2011) Bioresource Technol., 102: 11089-11096). Recently identified bacterial ligninases have been demonstrated to help improve the rate of degradation of lignocellulose (Bugg et al. (2011) Nat. Prod. Rept. 28: 1883-1896; Bugg et al. (2011) Curr. Opin. Biotechnol., 22: 394-400). Therefore, incorporation of both cellulase and ligninase enzymes may enable the cells to completely utilize the biomass (Zhang et al. (2012) Biotechnol. Adv. 30: 913-919).

Determination of Soluble Glycans/Xylans Released by Cells Displaying Six Cellulases on Dilute Acid Pretreated Corn Stover and Untreated Lignocellulose Substrates.

In order to more fully characterize the cellulolytic capabilities of cells displaying six cellulases in their cellulosomes, solubilized reducing sugars, glucose and xylose were analyzed from lignocellulose digestions using azide-treated cells. The standard 15 mg cell-displayed cellulase protein/g biomass was maintained in order to compare against the three cellulase displaying strain (TDA17) and commercial cellulase cocktails. Experiments were performed as described previously, in which cultures of cells displaying three or six enzymes were induced to express the cellulosomes. After reaching saturation, the cultures were collected and incubated with dilute acid pretreated corn stover or untreated lignocellulose substrates and samples taken periodically for 48 hours. Cells displaying six cellulase enzymes were able to release more than two-fold more reducing sugars from pretreated corn stover than cells displaying three enzymes (FIG. 9, panel B). Interestingly, increasing the number of enzymes enabled cell-displayed cellulosomes to reach 74% the degradative capacity of the Novozymes, Inc. cellulase cocktail Ctec2/Htec2. However, though 10 times more xylose and 2-3 times more glucose were released by the six-cellulase displaying strain as compared to the three-cellulase strain, the cells were unable to reach the same amount of glucose as that released by the cellulase cocktail (FIG. 9 panel C, and 9, panel D). This is most likely due to a β-glucosidase in the cocktail that is not present in the cellulosomes. Fascinatingly, the six-cellulase displaying cells were able to release ˜20% more xylose than the cellulase cocktail (FIG. 9, panel C). These data indicate that incorporation of additional endoglucanases and exoglucanases into the surface displayed cellulosome may further improve degradation.

Enzymatic degradation of untreated lignocellulosic substrates was also markedly better for the cells that displayed six cellulases. Overall, released soluble reducing sugars increased three to four fold over the amount solubilized by cells displaying only three enzymes (FIGS. 10 panel B, 11 panel B, and 12 panel B). This indicates that the enzymes that were found to be most abundant in native cellulosomes when C. thermocellum was cultured on lignocellulose may truly be essential in efficient degradation. The amount of xylose released by strain TDA22 improved three to four fold over strain TDA17, indicating that XynA may be quite important in solubilizing the hemicellulose (FIGS. 10, panel C, 11 panel C, and 12 panel C). Furthermore, glucose solubilized by the six cellulase displaying strain increased more than 10 fold on all substrates (FIGS. 10, panel D, 11 panel D, and 12 panel D). Interestingly, the Htec2/Ctec2 cocktail was less effective on untreated substrates than on pretreated (compare FIGS. 9 and 10). It is possible that since the biomass was untreated (minimal grinding, no incubation with acid and high heat), the surface area available may not have been as great for these cocktails as compared to pretreated substrates that are ground and treated with dilute acids.

Though these results indicate that the rationally chosen enzymes to be displayed in the six-cellulase cellulosome enabled a dramatic increase in lignocellulose hydrolysis, the substrates were not completely degraded. This could be due to a buildup of product inhibitors, such as cellobiose (Demain et al. (2005) MMBR 69: 124-154) or indicate the utility of displaying more enzymes to fully degrade the biomass.

Dionex analysis of Carbohydrates Released from Untreated Corn Stover by Three and Six Enzyme Displaying Cells.

In order to rationally determine what additional enzyme activities are required to improve degradation of lignocellulose, samples of strain TDA17 and TDA22 were azide-treated and incubated with untreated corn stover as described above. In addition, these strains were cultured on untreated corn stover to characterize the efficiency of lignocellulose saccharification when the cells are growing on it. Corn stover was chosen due to its use in cellulosic biofuel production and it was left untreated to determine what additional enzyme activities are required to efficiently digest untreated biomass.

To ascertain the types of carbohydrates that are released by cells displaying designer cellulosomes containing three or six cellulase enzymes, these strains were cultured in rich medium, induced to express the cellulosomes, and collected after the cells have reached saturation. Following expression, the cells were collected, washed, and resuspended in buffer containing 5 g/L untreated corn stover and 1% azide to make the cells inert. Following a 48 hour digestion at 37° C., the samples were autoclaved to stop enzymatic degradation, and the residual insoluble material was separated from the supernatant. The remaining biomass not solubilized by the cells was then completely saccharified using concentrated sulfuric acid and analyzed by Dionex high performance liquid chromatography (HPLC). It was determined that the untreated corn stover alone (not digested by the cells) consisted of 59% glucose, 15% xylose, 1% arabinose, and undetectable amounts of galactose and mannose (data not shown). As shown in Table 8 when strain TDA17 was exposed to biomass, 35% of it could be liberated, including 10% of the cellulose, 39% xylose, and 10% arabinose. However, when the lignocellulose was exposed to strain TDA22, more than half could be solubilized, resulting in 35% of the available glucose to be released into the supernatant, nearly half of the xylan and a third of the arabinan. Interestingly, arabinose solubilization was not expected as no arabinase was present in the cellulosome, but could have been released due to the hemicellulase activity of XynA. In addition, cells displaying three or six enzymes that were grown on the lignocellulose were able to degrade significantly higher amounts of the untreated lignocellulose than azide-killed cells. The increased degradation could likely be due to the constant removal of product inhibitors like cellobiose which would not be possible when the cells have been rendered metabolically inert (Demain et al. (2005) MMBR 69: 124-154). Furthermore, cells of strain TDA22 that were azide-killed or growing on untreated corn stover appeared to be almost as effective at solubilizing lignocellulose as the Ctec2/Htec2 cellulase cocktail (Table 8). In particular, more hemicellulose was removed by cells displaying cellulosomes than the Ctec2/Htec2 cocktail and could indicate that the cocktail may be catered toward the hydrolysis of cellulose and not hemicellulose. However, since the cellulase cocktail can solubilize nearly fifty percent more of the cellulose, the surface displayed cellulosomes may need to have additional endo- or exoglucanases incorporated into the complex to more efficiently work on the cellulose.

TABLE 8
% Monosaccharides solubilized after digestion of untreated corn stover

| Strain/cocktail | Total Biomass | Arabinose | Glucose | Xylose |
|---|---|---|---|---|
| tested | Solubilized | (1%)^{a} | (59%)^{a} | (15%)^{a} |
| Control (Biomass | 0 | 100 | 100 | 100 |
| only) | | | | |
| Control (TDA20 | 0 | 100 | 100 | 100 |
| cells plus | | | | |
| biomass) | | | | |
| TDA17 (azide | 35 ± 2.5 | 10 ± 1.4 | 10 ± 0.8 | 39 ± 2.3 |
| treated) | | | | |
| TDA21 (azide | 52 ± 4.2 | 36 ± 2.3 | 35 ± 2.1 | 48 ± 4.0 |
| treated) | | | | |
| TDA17 (growing | 54 ± 3.6 | 48 ± 1.1 | 46 ± 1.4 | 65 ± 4.5 |
| on biomass) | | | | |
| TDA21 (growing | 62 ± 5.2 | 48 ± 1.1 | 46 ± 1.4 | 65 ± 4.5 |
| on biomass) | | | | |
| Novozyme | 60 ± 7.1 | 14 ± 1.5 | 48 ± 1.6 | 51 ± 5.8 |
| Ctec2/Htec2 | | | | |

^{a}Percentages shown in parenthesis indicates prevalence in 5 g/L untreated corn stover sample.

Analysis of the types of carbohydrates solubilized by cells displaying three or six enzymes was also performed using Dionex HPLC in order to determine what additional enzyme activities may be required for better lignocellulose degradation. Overall, more glycans and xylans were released by cells displaying six enzymes (Tables 9 and 10). This is consistent with the amounts of carbohydrates that were remaining in the insoluble biomass pellet after saccharification by the cells. The data suggests that expansion of the cellulosome to include three new enzymes is essential for more efficient saccharification. However, a significant portion of the glycans and xylans solubilized by cells displaying three and six enzymes were long chain carbohydrates (greater than four sugars in length) and revealed that additional endoglucanases may be necessary to further degrade them. Interestingly, the Ctec2/Htec2 cocktail also had a significant amount of the solubilized long chain carbohydrates. This may indicate that product inhibition could indeed be partly responsible in the incomplete conversion of the carbohydrates to glucose when using azide-killed TDA22 cells or the Ctec2/Htec2 cocktail.

TABLE 9
Hexose sugars released from untreated corn stover (mM)

| Strain/cocktail | | | | | Other |
|---|---|---|---|---|---|
| tested | Glucose | Cellobiose | Cellotriose | Cellotetraose | Glycans |
| Control (Biomass | 0 | 0 | 0 | 0 | 0 |
| only) | | | | | |
| Control (TDA20 | 0 | 0 | 0 | 0 | 0 |
| cells plus | | | | | |
| biomass) | | | | | |
| TDA17 (azide | 0.3269 ± 0.0613 | 0.0272 ± 0.0093 | 0.0215 ± 0.0151 | ND^{a} | 1.0144 ± 0.2167 |
| treated) | | | | | |
| TDA21 (azide | 1.8903 ± 0.3166 | 0.0405 ± 0.0184 | 0.0012 ± 0.0062 | 0.0055 ± 0.0019 | 4.5328 ± 0.3814 |
| treated) | | | | | |
| TDA17 (growing | 0.0059 ± 0.0035 | 0.0045 ± 0.0019 | 0.0005 ± 0.0002 | ND^{a} | 0 |
| on biomass) | | | | | |
| TDA21 (growing | 0.0063 ± 0.0009 | 0.0069 ± 0.0038 | ND^{a} | ND^{a} | 0 |
| on biomass) | | | | | |
| Novozyme | 4.2960 ± 0.2100 | 0.0397 ± 0.0082 | 0.0027 ± 0.0005 | 0.0295 ± 0.0044 | 3.3121 ± 0.3426 |
| Ctec2/Htec2 | | | | | |

^{a}Not detected.

TABLE 10
Pentose sugars released from untreated corn stover (mM)

| Strain/cocktail | | | | | Other |
|---|---|---|---|---|---|
| tested | Xylose | Xylobiose | Xylotriose | Xylotetraose | xylans |
| Control (Biomass | 0 | 0 | 0 | 0 | 0 |
| only) | | | | | |
| Control (TDA20 | 0 | 0 | 0 | 0 | 0 |
| cells plus | | | | | |
| biomass) | | | | | |
| TDA17 (azide | 0.0255 ± 0.0094 | 0.1000 ± 0.0170 | 0.0467 ± 0.0217 | 0.0169 ± 0.0094 | 2.0509 ± 0.3426 |
| treated) | | | | | |
| TDA21 (azide | 0.0477 ± 0.0062 | 0.1413 ± 0.0489 | 0.0702 ± 0.0218 | 0.0323 ± 0.0090 | 2.0985 ± 0.2139 |
| treated) | | | | | |
| TDA17 (growing | 0.0670 ± 0.0296 | 0.0063 ± 0.0013 | 0.0047 ± 0.0019 | 0.0037 ± 0.0016 | 0 |
| on biomass) | | | | | |
| TDA21 (growing | 0.0114 ± 0.0083 | 0.0044 ± 0.0005 | 0.0032 ± 0.0009 | 0.0046 ± 0.0015 | 0 |
| on biomass) | | | | | |
| Novozyme | 1.3904 ± 0.1668 | 0.0486 ± 0.0141 | 0.0213 ± 0.0035 | 0.022 ± 0.0022 | 1.3681 ± 0.1142 |
| Ctec2/Htec2 | | | | | |

Further improvement of the cellulosome display system could include enlarging the complex to display an additional three enzymes, bringing the total number displayed to nine. To exploit the synergy of the enzymes currently displayed, Cel9G, an endoglucanase from C. cellulolyticum, can be incorporated because it has been demonstrated to work well together with Cel9E and Cel48F; a six-fold increase in digestion on wheat straw was observed when Cel9G is part of a complex containing Cel9E or Cel48F (Fierobe et al. (2001) J. Biol. Chem. 276: 21257-21261; Fierobe et al. (2005) J. Biol. Chem. 280: 16325-16334). Incorporation of another endoglucanase, C. cellulolyticum Cel8C could potentially enhance cellulose degradation as well to further produce more ends within the glycan chains that can be acted upon the exoglucanases present (Belaich et al. (1997) J. Biotechnol., 57: 3-14; Fierobe et al. (1993) Eur. J. Biochem./FEBS, 217: 557-565). In addition, another xylanase incorporated into the cellulosome could be beneficial to complete the removal of the hemicellulose. Since the incorporation of one xylanase enabled the solubilization of nearly 50% of the xylan, two xylanases may be able to function synergistically to completely remove the xylan. Xylanase XynZ from C. thermocellum has been shown to be abundant when cells were grown on cellulose, indicating that it could be essential in efficient xylan degradation (Gold and Martin (2007) J. Bacteriol., 189: 6787-6795; Raman et al. (2009) PloS One 4: e5271). Alternatively, addition of ligninases into the nine-cellulase cellulosome could prove to be critical in enhancing lignocellulose degradation. If the lignin could be removed easily by using these enzymes, there should no longer be a barrier for the cellulases and hemicellulases to function optimally. Incorporation of these additional enzymes should permit enhanced lignocellulose decomposition, and could potentially cause cells displaying cellulosomes to overcome the degradative capacity of the Ctec2/Htec2 cocktail.

### CONCLUSIONS

The ultimate goal of this work is to develop strains of bacteria that efficiently degrade lignocellulose into its component sugars. These strains could potentially replace purified cellulase cocktails and act as a “bag of enzymes”, with the distinct advantage of being easily recyclable between lignocellulose digestions. The B. subtilis strains that have been constructed demonstrate lignocellulose degradation that is comparable to that of the cellulase cocktails, on both untreated and pretreated biomass. The use of cells to degrade lignocellulose has many advantages over these cocktails. Most importantly, one would only have to maintain bacterial cultures as opposed to the production and purification of multiple cellulase enzymes. In addition, potential cost savings could be realized due to the easy recyclability of cells displaying cellulases; cells displaying cellulases have recently been demonstrated to be functional after five digestion cycles (Matano et al. (2012) Bioresour Technol. 135: 403-409).

Another potential application and long-term goal of this work is to create consolidated bioprocessing (CBP) microorganisms that can directly convert lignocellulose into useful biofuels and other high-value biocommodities. A CBP could also potentially dramatically reduce production costs by combining all steps into a single organism, reducing the necessity to first hydrolyze the lignocellulose into fermentable sugars and then feed to yeast for fermentation (Mazzoli et al. (2012) Trends Biotechnol., 30: 111-119; Olson et al. (2012) Curr. Opin. Biotechnol., 23: 396-405). Work described in this thesis has created strains of bacteria that can effectively utilize lignocellulose as a source for growth, suggesting that this type of system could be useful in the construction of a CBP organism. In addition, B. subtilis has been demonstrated to be able to produce ethanol and other biofuels from glucose (Romero et al. (2007) Appl. Environ. Microbiol. 73: 5190-5198). It therefore seems feasible that engineering metabolic pathways to produce isobutanol, or other biocommodities, could be integrated into the cellulolytic strains of B. subtilis created. This result would be interesting because only a small number of CBPs created have demonstrated the ability to produce biofuels directly from lignocellulosic substrates (Bokinsky et al. (2011) Proc. Natl. Acad. Sci. USA, 108: 19949-19954). For these reasons, the strains developed in this thesis will likely be further optimized to produce chemicals of great social value.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant modified Gram-positive bacterium that displays on its surface a minicellulosome comprising two or more cellulolytic enzymes, wherein said bacterium comprises:
a protein encoding two or more cohesin domains wherein said protein is covalently linked to the surface of said microorganism, and wherein each of said cohesin domains is docked to a dockerin attached to a cellulolytic enzyme; and
said bacterium comprises one or more constructs that encode said dockerin(s) attached to said cellulolytic enzyme(s); and
wherein said minicellulosome is self-assembled by said bacterium.

2. The bacterium of claim 1, wherein said bacterium grows on untreated plant biomass.

3. The bacterium of claim 1, wherein said bacterium grows on lignocellulose as the sole carbon source.

4. The bacterium of claim 1, wherein said minicellulosome comprises at least three cellulolytic enzymes and all of said enzymes are encoded by said bacterium.

5. The bacterium of claim 1, wherein said protein encoding two or more cohesin domains comprises a secretory signal sequence at the N-terminus and a cell wall sorting signal (CWSS) at the carboxyl terminus.

6. The bacterium of claim 5, wherein said cell wall sorting signal comprises an LPXTG motif.

7. The bacterium of claim 5, wherein said cell wall sorting signal comprises a sequence shown in Table 1.

8. The bacterium of claim 5, wherein said cell wall sorting signal comprises a CWSS from Staphylococcus aureus fibronectin binding protein B.

9. The bacterium of claim 5, wherein said secretory signal sequence comprises a B. subtilis phrC secretory signal or homologues thereof and/or a secretion signal derived from B. subtilis phrC.

10. (canceled)

11. The bacterium of claim 1, wherein said protein encoding two or more cohesin domains encodes a carbohydrate binding module (CBM).

12. (canceled)

13. The bacterium of claim 1,
wherein said two or more cohesin domains are type I cohesin modules.

14. (canceled)

15. The bacterium of claim 1, wherein
said two or more cohesin domains comprises a cohesin domain from an organism selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus; and/or
said dockerins comprise one or more dockerin domains from organism(s) selected from the group consisting of Clostridium thermocellum, Clostridium cellulolyticum, Ruminococcus flavefaciens, C. cellulovorans, C. acetobutylicum, C. josui, C. papyrosolvens, A. cellulolyticus, and R. albus.

16-21. (canceled)

22. The bacterium of claim 1, wherein said cellulolytic enzyme(s) comprise one or more enzymes selected from the group consisting of an endocellulase, an exocellulase, a beta-glucosidase (cellobiase), an oxidative cellulase, a xylanase, a hemicellulase, a lichenase, a chitenase, and a cellulose phosphorylase.

23. The bacterium of claim 1,
wherein said minicellulosome comprises at least two different cellulolytic enzymes or at least three different cellulolytic enzymes.

24. (canceled)

25. The bacterium of claim 1, wherein said minicellulosome comprises at least one endoglucanase and/or at least one exoglucanase.

26-27. (canceled)

28. The bacterium of claim 1,
wherein said minicellulosome comprises Clostridium cellulolyticum endoglucanase Cel5A, and/or C. cellulolyticum endoglucanase Cel48, and/or C. cellulolyticum exoglucanase Cel9E.

29-30. (canceled)

31. The bacterium of claim 1, wherein said Gram-positive bacterium comprises a Gram-positive bacterium that encodes a sortase.

32-38. (canceled)

39. A method of degrading cellulosic biomass into fermentable sugars, said method comprising:
contacting said cellulosic biomass with a bacterium of claim 1, under conditions in which said bacteria partially or fully degrade cellulose in said cellulosic biomass to form one or more fermentable sugars.

40-45. (canceled)

46. A consolidated bioreactor for the conversion of a lignocellulosic biomass into bioethanol said bioreactor comprising:
a culture system that cultures bacteria of claim 1 under conditions in which said bacteria partially or fully degrade cellulose in said lignocellulosic biomass to form one or more fermentable sugars; and
a culture system that ferments said sugars to form a biofuel.

47. A method of identifying cellulolytic enzyme combinations that enhance degradation of a particular substrate said method comprising:
providing a plurality of recombinant bacteria of claim 1, wherein said bacteria each display at least two cellulolytic enzymes and different bacteria display different enzymes;
contacting said substrate with said bacteria; and
selecting bacteria that show enhanced degradation of said substrate and/or improved growth on said substrate.

48. A method of identifying cellulolytic enzyme variants that enhance degradation of a particular substrate said method comprising:
providing a plurality of recombinant bacteria of claim 1, wherein said bacteria each display at least one cellulolytic enzyme variant and different bacteria display different cellulolytic enzyme variants;
contacting said substrate with said bacteria; and
selecting bacteria that show enhanced degradation of said substrate and/or improved growth on said substrate.

49-51. (canceled)
